SEQUENCE LISTING (12 sequences)

SEQ ID NO: 1 - DNA, 2901 bp, Artificial Sequence
  Medicago sativa phosphoenolpyruvate carboxylase ppc*
atggcaaacaaaatggaaaagatggcaagcattgacgcgcaactgcgccagttggtcccggcaaaagtcagcgaggacgacaaattgattgaatacgatgctctgttgctggaccgctttctggacattctgcaagatctgcatggcgaggatctgaaggattcggttcaggaagtttacgaactgtctgcggagtatgagcgtaagcatgacccgaagaagctggaagagctgggtaacttgattacgagctttgacgcgggcgacagcattgtcgtggcgaaatcgttctctcatatgctgaatctggcgaacctggccgaagaagttcaaattgctcaccgccgtcgtaacaagctgaagaagggtgattttcgtgatgagagcaatgcgaccaccgagtccgatattgaggagactctgaagaaactggttttcgacatgaagaagtctccgcaagaagtgtttgacgcgttgaagaatcagaccgtggacctggtgctgacggcacatcctacccagagcgttcgccgttccctgctgcaaaagcatggtcgtgttcgtaattgcttgagccagctgtatgcgaaagacattaccccggatgacaaacaagagctggacgaggcactgcagcgtgaaatccaggcagcgttccgtaccgatgaaatcaaacgtaccccgccgaccccacaagacgaaatgcgtgctggcatgagctatttccacgaaaccatctggaagggcgtcccgaagttcctgcgtcgcgtggacaccgcgttgaagaacatcggcattaacgaacgcgtgccgtataacgccccgctgattcaattcagcagctggatgggtggcgaccgtgacggcaatccgcgtgttacgccagaagtgacccgtgatgtttgtctgctggcgcgtatgatggcggcgaatttgtactatagccagattgaagatctgatgtttgagctgtctatgtggcgctgtaatgatgagttgcgtgtgcgtgccgaagaactgcaccgcaatagcaagaaagacgaagttgccaagcactacatcgagttctggaagaagatcccgttgaacgagccgtaccgtgttgttctgggtgaggtccgcgataagctgtatcgcacccgtgagcgcagccgttatctgctggcacacggttattgcgaaattccggaggaggcgacctttaccaacgtggatgaatttctggaaccgctggagctgtgttatcgtagcctgtgcgcgtgcggtgaccgcgcgattgcggacggttctttgctggatttcctgcgccaggtgagcacgtttggtctgagcctggtccgtctggatatccgtcaggaatcggaccgccatacggatgtgatggacgctattaccaaacacctggaaattggcagctaccaggagtggagcgaggagaaacgtcaagagtggctgctgagcgagctgatcggtaagcgtccgctgttcggtccagatctgccgcaaaccgacgaaatccgcgacgttctggacacctttcgtgtgattgccgaactgccgagcgacaacttcggcgcgtacattatctccatggccaccgccccgagcgatgtcctggcagtcgagctgctgcaacgcgaatgtaaggtccgtaacccgttgcgcgtggttccgctgtttgaaaagctggatgacctggagagcgcaccggccgcactggctcgtctgtttagcattgactggtacattaaccgtattgatggtaaacaggaagtgatgattggttactccgacagcggtaaagatgcgggtcgttttagcgccgcatggcagctgtacaaggcacaagaagatctgatcaaggttgcacagaagttcggcgttaaactgaccatgttccacggtcgcggtggtacggttggccgtggtggcggcccaacccacctggcgattctgagccaaccgccggagactatccatggttccttgcgtgtcaccgtccagggcgaagtgattgagcaaagcttcggcgaggaacatctgtgctttcgcaccctgcagcgttttacggccgcgactttggaacacggcatgcgtccgccatccagcccaaagccagaatggcgtgcgctgatggaccaaatggcggttatcgcgaccgaggagtatcgcagcattgtgttcaaagagccgcgttttgtggagtatttccgtttggcaacgccggagatggagtacggccgcatgaatatcggcagccgtccggcaaaacgtcgcccgtccggcggcatcgagacgctgcgtgccatcccgtggattttcgcgtggacgcagacccgtttccatttgccggtgtggctgggtttcggtgccgcctttcgtcaagtcgtgcagaaggacgtgaagaatctgcatatgctgcaggagatgtacaaccagtggccgttctttcgtgtcaccattgatctggtggaaatggtctttgcgaaaggtgatccgggcatcgcggcgttgaatgaccgtctgctggtttccaaagacctgtggccttttggtgaacagctgcgtagcaagtacgaggaaaccaagaaactgctgttgcaagttgcggcgcacaaggaggtgctggaaggtgacccttatctgaagcaacgcctgcgtctgcgtgactcgtacatcacgaccctgaatgtctttcaggcgtataccctgaagcgtatccgtgacccgaattacaaagtggaagttcgccctccgatcagcaaggagagcgcggagactagcaaaccagcggacgaactggtcaccctgaatccgacctcggagtatgctccgggtttggaagatacgctgattctgacgatgaagggtatcgcggctggcatgcagaacacgggctaa

SEQ ID NO: 2 - protein, 966 aa, Artificial Sequence
  Medicago sativa phosphoenolpyruvate carboxylase ppc*
MANKMEKMASIDAQLRQLVPAKVSEDDKLIEYDALLLDRFLDILQDLHGEDLKDSVQEVYELSAEYERKHDPKKLEELGNLITSFDAGDSIVVAKSFSHMLNLANLAEEVQIAHRRRNKLKKGDFRDESNATTESDIEETLKKLVFDMKKSPQEVFDALKNQTVDLVLTAHPTQSVRRSLLQKHGRVRNCLSQLYAKDITPDDKQELDEALQREIQAAFRTDEIKRTPPTPQDEMRAGMSYFHETIWKGVPKFLRRVDTALKNIGINERVPYNAPLIQFSSWMGGDRDGNPRVTPEVTRDVCLLARMMAANLYYSQIEDLMFELSMWRCNDELRVRAEELHRNSKKDEVAKHYIEFWKKIPLNEPYRVVLGEVRDKLYRTRERSRYLLAHGYCEIPEEATFTNVDEFLEPLELCYRSLCACGDRAIADGSLLDFLRQVSTFGLSLVRLDIRQESDRHTDVMDAITKHLEIGSYQEWSEEKRQEWLLSELIGKRPLFGPDLPQTDEIRDVLDTFRVIAELPSDNFGAYIISMATAPSDVLAVELLQRECKVRNPLRVVPLFEKLDDLESAPAALARLFSIDWYINRIDGKQEVMIGYSDSGKDAGRFSAAWQLYKAQEDLIKVAQKFGVKLTMFHGRGGTVGRGGGPTHLAILSQPPETIHGSLRVTVQGEVIEQSFGEEHLCFRTLQRFTAATLEHGMRPPSSPKPEWRALMDQMAVIATEEYRSIVFKEPRFVEYFRLATPEMEYGRMNIGSRPAKRRPSGGIETLRAIPWIFAWTQTRFHLPVWLGFGAAFRQVVQKDVKNLHMLQEMYNQWPFFRVTIDLVEMVFAKGDPGIAALNDRLLVSKDLWPFGEQLRSKYEETKKLLLQVAAHKEVLEGDPYLKQRLRLRDSYITTLNVFQAYTLKRIRDPNYKVEVRPPISKESAETSKPADELVTLNPTSEYAPGLEDTLILTMKGIAAGMQNTG

SEQ ID NO: 3 - DNA, 1362 bp, Artificial Sequence
  Clostridium kluyveri succinate semialdehyde dehydrogenase sucD*
atgtccaacgaggttagcattaaggagctgattgagaaggcgaaagtggcgcagaaaaagctggaagcgtatagccaagagcaagttgacgttctggtcaaggcgctgggtaaagttgtgtacgacaacgccgagatgttcgcgaaagaggcggtggaggaaaccgagatgggtgtttacgaggataaagtggctaaatgtcatctgaaatctggtgcaatctggaatcacattaaagataagaaaaccgttggtattatcaaggaagaaccggagcgtgcgctggtgtacgtcgcgaagcctaaaggtgttgtggcggcgacgacccctatcaccaatcctgtggttaccccgatgtgtaacgcgatggcagcaattaaaggtcgcaacaccatcattgtcgccccgcatccgaaggcgaagaaggtgagcgcgcacaccgtggagctgatgaatgcagaactgaaaaagttgggtgcgccggaaaacattatccagatcgttgaagccccaagccgtgaagcagccaaggagttgatggagagcgcagacgtggttatcgccacgggtggcgcaggccgtgttaaagcagcgtactcctccggccgtccggcatacggtgtcggtccgggcaattctcaggtcattgtcgataagggttacgattataacaaagctgcccaggacatcattaccggccgcaagtatgacaacggtatcatttgcagctctgagcagagcgtgatcgcaccggcggaggactacgacaaggtcatcgcggctttcgtcgagaatggcgcgttctatgtcgaggatgaggaaactgtggagaaattccgtagcacgctgttcaaggatggcaagatcaatagcaaaatcatcggtaaatccgtgcagatcatcgctgacctggctggtgtcaaggtgccggaaggcaccaaggtgatcgtgttgaagggcaagggtgccggtgaaaaggacgttctgtgcaaggagaaaatgtgcccggtcctggttgccctgaaatatgacacctttgaggaggcggtcgagatcgcgatggccaactatatgtacgagggtgcgggccataccgccggtatccacagcgataacgacgagaatatccgctacgcgggtacggtgctgccaatcagccgtctggttgtcaaccagccagcaactacggccggtggtagctttaacaatggttttaatccgaccaccaccttgggctgcggtagctggggccgtaactccattagcgagaacctgacgtatgagcatctgattaatgtcagccgtattggctatttcaataaggaggcaaaagttcctagctacgaggagatctggggttaa

SEQ ID NO: 4 - protein, 453 aa, Artificial Sequence
  Clostridium kluyveri succinate semialdehyde dehydrogenase sucD*
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 5 - DNA, 870 bp, Artificial Sequence
  Arabidopsis thaliana succinic semialdehyde reductase ssaRAt*
atggaagtaggttttctgggtctgggcattatgggtaaagctatgtccatgaacctgctgaaaaacggtttcaaagttaccgtgtggaaccgcactctgtctaaatgtgatgaactggttgaacacggtgcaagcgtgtgcgagtctccggctgaggtgatcaagaaatgcaaatacacgatcgcgatgctgagcgatccgtgtgcagctctgtctgttgttttcgataaaggcggtgttctggaacagatctgcgagggtaagggctacatcgacatgtctaccgtcgacgcggaaactagcctgaaaattaacgaagcgatcacgggcaaaggtggccgttttgtagaaggtcctgttagcggttccaaaaagccggcagaagacggccagctgatcatcctggcagcaggcgacaaagcactgttcgaggaatccatcccggcctttgatgtactgggcaaacgttccttttatctgggtcaggtgggtaacggtgcgaaaatgaaactgattgttaacatgatcatgggttctatgatgaacgcgtttagcgaaggtctggtactggcagataaaagcggtctgtctagcgacacgctgctggatattctggatctgggtgctatgacgaatccgatgttcaaaggcaaaggtccgtccatgactaaatccagctacccaccggctttcccgctgaaacaccagcagaaagacatgcgtctggctctggctctgggcgacgaaaacgctgttagcatgccggtcgctgcggctgcgaacgaagccttcaagaaagcccgtagcctgggcctgggcgatctggacttttctgctgttatcgaagcggtaaaattctctcgtgaataa

SEQ ID NO: 6 - protein, 289 aa, Artificial Sequence
  Arabidopsis thaliana succinic semialdehyde reductase ssaRAt*
MEVGFLGLGIMGKAMSMNLLKNGFKVTVWNRTLSKCDELVEHGASVCESPAEVIKKCKYTIAMLSDPCAALSVVFDKGGVLEQICEGKGYIDMSTVDAETSLKINEAITGKGGRFVEGPVSGSKKPAEDGQLIILAAGDKALFEESIPAFDVLGKRSFYLGQVGNGAKMKLIVNMIMGSMMNAFSEGLVLADKSGLSSDTLLDILDLGAMTNPMFKGKGPSMTKSSYPPAFPLKHQQKDMRLALALGDENAVSMPVAAAANEAFKKARSLGLGDLDFSAVIEAVKFSRE

SEQ ID NO: 7 - DNA, 1053 bp, Artificial Sequence
  Aspergillus terreus succinic semialdehyde reductase ssaRAt2*
atgccactggttgctcaaaatccactgccacgtgctattctgggtctgatgactttcggtccgagcgaaagcaaaggtgcgcgtatcacttccctggatgagtttaacaagtgcctggattacttccagcagcagggcttccaggaaatcgataccgcgcgcatctacgtcggcggtgaacaggaggcattcacggcgcaggcaaagtggaaagaacgcggcctgacgctggcgactaagtggtatccgcagtacccgggtgcgcacaaaccggatgtcctgcgtcagaacctggagctgtccctgaaagaactgggcacgaaccaggtcgatatcttctatctgcacgccgcggatcgttctgtgccgttcgcggaaactctggaaactgttaacgaactgcacaaagaaggcaaatttgttcagctgggtctgtctaactacaccgctttcgaagtagctgaaatcgtgaccctgtgtaacgagcgtggttgggttcgtccgactatctaccaggcgatgtataacgctatcacccgtaacatcgaaactgaactgatcccggcgtgcaagcgttacggtattgacattgttatctacaacccactggcgggtggcctgttcagcggcaaatacaaagcacaggacatcccggctgaaggtcgttacagcgaccaatcttccatgggccagatgtaccgcaaccgttactttaaggacgcaacctttgacgctctgcgcctgatcgaaccggttgttgcgaagcacggcctgacgatgccggaaaccgcgttccgctgggtccaccaccactccgcactgaacatggaagatggcggccgtgacggcatcattctgggtgtaagcagcctggctcagctggaaaacaacctgaaagacattcagaaaggtccgctgccgcaggaggttgtagacgtcctggatcaggcttggctggtggctaagccgacggctccaaactactggcatctggacctgaaatacacgtacgacacccaggaagctctgttcaaaccgaaatctaaggcgtaa

SEQ ID NO: 8 - protein, 357 aa, Artificial Sequence
  Aspergillus terreus succinic semialdehyde reductase ssaRAt2*
MPLVAQNPLPRAILGLMTFGPSESKGARITSLDEFNKCLDYFQQQGFQEIDTARIYVGGEQEAFTAQAKWKERGLTLATKWYPQYPGAHKPDVLRQNLELSLKELGTNQVDIFYLHAADRSVPFAETLETVNELHKEGKFVQLGLSNYTAFEVAEIVTLCNERGWVRPTIYQAMYNAITRNIETELIPACKRYGIDIVIYNPLAGGLFSGKYKAQDIPAEGRYSDQSSMGQMYRNRYFKDATFDALRLIEPVVAKHGLTMPETAFRWVHHHSALNMEDGGRDGIILGVSSLAQLENNLKDIQKGPLPQEVVDVLDQAWLVAKPTAPNYWHLDLKYTYDTQEALFKPKSKAAVKFSRE

SEQ ID NO: 9 - DNA, 1104 bp, Artificial Sequence
  Mus musculus succinic semialdehyde reductase ssaRMm*
atgctgcgtgctgcttctcgtgctgttggtcgtgctgctgtacgttccgctcaacgttctggtactagcgttggccgtccgctggcgatgtcccgtccaccgccgcctcgcgcagctagcggtgccccgctgcgtccggcaaccgtactgggcactatggagatgggtcgtcgcatggacgcttctgcatccgcggcaagcgttcgtgcgttcctggaacgtggccatagcgaactggataccgctttcatgtattgcgacggtcagtccgaaaatatcctgggtggcctgggcctgggtctgggctccggtgattgtaccgttaaaattgcgaccaaggcgaacccttgggagggcaagagcctgaagccggattctgtgcgttctcagctggagacttctctgaaacgtctgcagtgtccgcgcgtagacctgttctatctgcatgcgccggaccacagcactccggtagaggaaactctgcgtgcgtgtcatcagctgcaccaggaaggcaagttcgtcgaactgggtctgtctaactacgcatcttgggaagtggcagaaatctgtacgctgtgtaagtctaatggttggatcctgccaaccgtgtaccagggcatgtacaacgctaccacccgccaggtagaagcagaactgctgccgtgcctgcgtcacttcggcctgcgcttttacgcttacaacccgctggcgggtggtctgctgacgggcaaatacaagtatgaagataaagatggtaaacaaccggtcggtcgtttctttggtaacaactgggccgaaacctaccgtaatcgcttctggaaagagcaccactttgaagcgatcgcactggttgaaaaagcgctgcagacgacttatggcactaacgcgccgcgtatgacctccgctgcgctgcgttggatgtaccaccatagccagctgcagggtactcgcggcgatgccgttatcctgggcatgagctccctggaacagctggaacagaacctggccgcgactgaagagggcccgctggaaccggcagttgtcgaagcttttgaccaggcatggaacatggtggcgcacgaatgtccaaactatttccgctaa

SEQ ID NO: 10 - protein, 367 aa, Artificial Sequence
  Mus musculus succinic semialdehyde reductase ssaRMm*
MLRAASRAVGRAAVRSAQRSGTSVGRPLAMSRPPPPRAASGAPLRPATVLGTMEMGRRMDASASAASVRAFLERGHSELDTAFMYCDGQSENILGGLGLGLGSGDCTVKIATKANPWEGKSLKPDSVRSQLETSLKRLQCPRVDLFYLHAPDHSTPVEETLRACHQLHQEGKFVELGLSNYASWEVAEICTLCKSNGWILPTVYQGMYNATTRQVEAELLPCLRHFGLRFYAYNPLAGGLLTGKYKYEDKDGKQPVGRFFGNNWAETYRNRFWKEHHFEAIALVEKALQTTYGTNAPRMTSAALRWMYHHSQLQGTRGDAVILGMSSLEQLEQNLAATEEGPLEPAVVEAFDQAWNMVAHECPNYFR

SEQ ID NO: 11 - DNA, 1821 bp, Artificial Sequence
  Pseudomonas putida/Ralstonia eutropha JMP134 Polyhydroxyalkanoate synthase fusion protein phaC3/C1
atgactagaaggaggtttcatatgagtaacaagaacaacgatgagctggcgacgggtaaaggtgctgctgcatcttctactgaaggtaaatctcagccgtttaaattcccaccgggtccgctggacccggccacttggctggaatggagccgtcagtggcaaggtccggagggcaatggcggtaccgtgccgggtggctttccgggtttcgaagcgttcgcggcgtccccgctggcgggcgtgaaaatcgacccggctcagctggcagagatccagcagcgttatatgcgtgatttcaccgagctgtggcgtggtctggcaggcggtgacaccgagagcgctggcaaactgcatgaccgtcgcttcgcgtccgaagcgtggcacaaaaacgcgccgtatcgctatactgcggcattttacctgctgaacgcacgtgcactgacggaactggctgatgcagtagaagcggatccgaaaacccgtcagcgtatccgttttgcggtttcccagtgggtagatgctatgagcccggctaacttcctggccaccaacccggacgctcagaaccgtctgatcgagagccgtggtgaaagcctgcgtgccggcatgcgcaatatgctggaagatctgacccgcggtaaaatttcccaaaccgatgagactgccttcgaagtaggccgtaacatggcagttaccgaaggtgctgtggtattcgaaaacgagttcttccagctgctgcagtacaaacctctgactgacaaagtatacacccgtccgctgctgctggtaccgccgtgcattaacaagttctatattctggacctgcagccggaaggttctctggtccgttacgcagtcgaacagggtcacactgtattcctggtgagctggcgcaatccagacgctagcatggctggctgtacctgggatgactatattgaaaacgcggctatccgcgccatcgaggttgtgcgtgatatcagcggtcaggacaagatcaacaccctgggcttttgtgttggtggcacgatcatctccactgccctggcggtcctggccgcccgtggtgagcacccggtggcctctctgaccctgctgactaccctgctggacttcaccgatactggtatcctggatgttttcgtggacgagccacacgttcagctgcgtgaggcgactctgggcggcgccagcggcggtctgctgcgtggtgtcgagctggccaataccttttccttcctgcgcccgaacgacctggtttggaactacgttgttgacaactatctgaaaggcaacaccccggtacctttcgatctgctgttctggaacggtgatgcaaccaacctgcctggtccatggtactgttggtacctgcgtcatacttacctgcagaacgaactgaaagagccgggcaaactgaccgtgtgtaacgaacctgtggacctgggcgcgattaacgttcctacttacatctacggttcccgtgaagatcacatcgtaccgtggaccgcggcttacgccagcaccgcgctgctgaagaacgatctgcgtttcgtactgggcgcatccggccatatcgcaggtgtgatcaaccctcctgcaaagaaaaagcgttctcattggaccaacgacgcgctgccagaatccgcgcaggattggctggcaggtgctgaggaacaccatggttcctggtggccggattggatgacctggctgggtaaacaagccggtgcaaaacgtgcagctccaactgaatatggtagcaagcgttatgctgcaatcgagccagcgccaggccgttacgttaaagcgaaagcataa

SEQ ID NO: 12 - protein, 599 aa, Artificial Sequence
  Pseudomonas putida/Ralstonia eutropha JMP134 Polyhydroxyalkanoate synthase fusion protein phaC3/C1
MSNKNNDELATGKGAAASSTEGKSQPFKFPPGPLDPATWLEWSRQWQGPEGNGGTVPGGFPGFEAFAASPLAGVKIDPAQLAEIQQRYMRDFTELWRGLAGGDTESAGKLHDRRFASEAWHKNAPYRYTAAFYLLNARALTELADAVEADPKTRQRIRFAVSQWVDAMSPANFLATNPDAQNRLIESRGESLRAGMRNMLEDLTRGKISQTDETAFEVGRNMAVTEGAVVFENEFFQLLQYKPLTDKVYTRPLLLVPPCINKFYILDLQPEGSLVRYAVEQGHTVFLVSWRNPDASMAGCTWDDYIENAAIRAIEVVRDISGQDKINTLGFCVGGTIISTALAVLAARGEHPVASLTLLTTLLDFTDTGILDVFVDEPHVQLREATLGGASGGLLRGVELANTFSFLRPNDLVWNYVVDNYLKGNTPVPFDLLFWNGDATNLPGPWYCWYLRHTYLQNELKEPGKLTVCNEPVDLGAINVPTYIYGSREDHIVPWTAAYASTALLKNDLRFVLGASGHIAGVINPPAKKKRSHWTNDALPESAQDWLAGAEEHHGSWWPDWMTWLGKQAGAKRAAPTEYGSKRYAAIEPAPGRYVKAKA

(19) United States
(12) Patent Application Publication
(10) Pub. No.: US 2014/0114082 A1
(43) Pub. Date: Apr. 24, 2014

(54) Biorefinery Process For THF Production

(71) Applicants: Johan van Walsem, Acton, MA (US); Erik Anderson, Somerville, MA (US); John Licata, Groveland, MA (US); Kevin A. Sparks, Scituate, MA (US); William R. Farmer, Concord, MA (US); Christopher Mirley, Winthrop, MA (US); Jeffrey A. Bickmeier, Arlington, MA (US); Ann D'Ambruoso, Waltham, MA (US); Frank Skraly, Watertown, MA (US); Thomas M. Ramseier, Newton, MA (US); M. S. Sivasubramanian, Wayland, MA (US); Oliver P. Peoples, Arlington, MA (US); Yossef Shabtai, Concord, MA (US)

(72) Inventors: Johan van Walsem, Acton, MA (US); Erik Anderson, Somerville, MA (US); John Licata, Groveland, MA (US); Kevin A. Sparks, Scituate, MA (US); William R. Farmer, Concord, MA (US); Christopher Mirley, Winthrop, MA (US); Jeffrey A. Bickmeier, Arlington, MA (US); Ann D'Ambruoso, Waltham, MA (US); Frank Skraly, Watertown, MA (US); Thomas M. Ramseier, Newton, MA (US); M. S. Sivasubramanian, Wayland, MA (US); Oliver P. Peoples, Arlington, MA (US); Yossef Shabtai, Concord, MA (US)

(73) Assignee: METABOLIX, INC., Cambridge, MA (US)

(21) Appl. No.: 14/125,045

(22) PCT Filed: Jun. 8, 2012
(86) PCT No.: PCT/US2012/041512

§ 371 Date: Dec. 9, 2013

Related U.S. Application Data
(60) Provisional application No. 61/494,650, filed on Jun. 8, 2011.

(51) Int. Cl. C12P 17/04

(52) U.S. Cl. CPC C12P 17/04
USPC 549/509; 435/126; 562/513

(57) ABSTRACT

Processes and methods for making biobased tetrahydrofuran products from renewable carbon resources are described herein.

### RELATED APPLICATION

This application claims the benefit of U.S. Provisional Application No. 61/494,650, filed on Jun. 8, 2011. The entire teachings of the above application are incorporated herein by reference.

### BACKGROUND OF THE INVENTION

With dwindling petroleum resources, increasing energy prices, and growing environmental concerns, development of energy efficient biorefinery processes to produce biobased chemicals from renewable, low cost, carbon resources offers a unique solution to overcoming the increasing limitations of petroleum-based chemicals.

One chemical with wide industrial uses that could be manufactured using a biorefinery process is tetrahydrofuran (THF). Tetrahydrofuran is a highly flammable, clear, colorless liquid, used in the production of polyurethane stretch fibers (spandex), coatings, adhesives, printing inks, magnetic tape and as a reaction solvent. THF ranks among the most polar organic solvents and offers outstanding solvent strength for many organic substances. A recent report issued by Global Indrustry Analysts GIA stated that the global market demand for tetrahydrofuran (THF) is projected to exceed 800 thousand tons/yr by 2017. Growth opportunities in the tetrahydrofuran market stem mainly from the increasing demand for polytetramethylene ether glycol (PTMEG), which is a major raw material used in the manufacture of spandex fibers. In addition, escalating demand for green technologies worldwide is expected to trigger an industry shift towards THF produced through bio-based manufacturing process.

Tetrahydrofuran can be manufactured by several different chemical processes. The most widely used industrial process involves the acid-catalyzed dehydration of 1,4-butanediol (BDO). The 1,4-butanediol is derived from carbonylation of acetylene followed by hydrogenation (Reppe Process) or also by conversion of maleic acid to its corresponding ester using acid resin followed by catalytic vapor phase hydrogenation to BDO (Davy Process). Both processes are based on nonrenewable, petroleum-based feedstocks. In the case of the Reppe Process, the feedstock is highly flammable and explosive which therefore requires special equipment in order to process it. The Davy Process is somewhat safer from a materials handling perspective, however it is a multistep reaction where the final product yield is highly dependent on the efficiencies of the various catalysts used in the process and requires significant energy input at each conversion stage.

Processes for manufacturing THF (or a derivative of THF, 2-methyl-THF) from renewable resources like corn cobs and sugar cane biomass are also known. In the case of THF production, furan derived from dehydration and decarboxylation of 5-carbon sugars compounds found in the hemicellulose fraction of biomass waste, is catalytically hydrogenated to form the THF. Likewise, 2-methyl-THF is obtained from catalytic hydrogenation of furfural, also produced by the dehydration of pentose sugar compounds found in corn stalks, corn cobs and peanut husks. Both of the above processes use strong mineral acids to first degrade the biomass and then dehydrate the 5-carbon sugars. As such, these are not very environmentally friendly processes.

Therefore there is a need to produce biobased chemicals such as THF from renewable resources that is simple, energy efficient, produces high purity THF in high yield and is environmentally friendly.

### SUMMARY OF THE INVENTION

The invention generally relates to an integrated biorefinery processes for producing high purity, high yielding, biobased tetrahydrofuran (THF) product from renewable carbon resources. In one aspect, a process for the production of tetrahydrofuran (THF) product from a genetically engineered microbial biomass metabolizing glucose or any other renewable feedstock to produce 4-hydroxybutyrate homopolymer (P4HB) inside the microbial cells, followed by controlled heating of the biomass containing P4HB with a first catalyst forming gamma-butyrolactone (GBL) vapor, then hydrogenation of the GBL vapor in the presence of a second catalyst is described. In a certain aspect, a process for production of a biobased tetrahydrofuran product by combining a genetically engineered biomass and a first catalyst, wherein the biomass comprises a poly-4-hydroxybutyrate; heating the biomass with the first catalyst to convert the poly 4-hydroxybutyrate to gamma-butyrolactone vapor with a yield of the gamma-butyrolactone vapor is at least 85%; and hydrogenating the gamma-butyrolactone vapor using a second catalyst to produce the tetrahydrofuran product is described.

The level of P4HB in the biomass should be greater than 10% by weight of the total biomass. The advantages of this bioprocess are that it uses a renewable carbon source as the feedstock material, the genetically engineered microbe produces P4HB in very high yield without adverse toxicity effects to the host cell (which could limit process efficiency) and when the biomass is combined with a first catalyst, heated and then hydrogenated in the vapor phase with a second catalyst, is capable of producing biobased THF in high yield and with high purity. Other advantages include in situ conversion with no requirement for costly downstream purification, no toxic metal catalysts and the high conversion rate of a product with high renewable content.

In certain aspects, a recombinant engineered P4HB biomass from a host organism serves as a renewable source for converting 4-hydroxybutyrate homopolymer to the useful chemical tetrathydrofuran (THF). In some embodiments, a source of the renewable feedstock is selected from glucose, fructose, sucrose, arabinose, maltose, lactose, xylose, fatty acids, vegetable oils, and biomass derived synthesis gas or a combination of two or more of these. The recombinantly produced P4HB biomass is heat treated in the presence of a first catalyst to produce gamma-butyrolactone (GBL) vapor, then hydrogenated in the presence of a second vapor phase catalyst to produce tetrathydrofuran (THF). In other embodiments, the P4HB biomass is dried prior to combining with the first catalyst.

In other embodiments, the GBL vapor is processed in situ without first recovering it in liquid form. This greatly improves the energetics of the process by avoiding the energy intensive operations of condensing GBL and subsequently vaporizing the same GBL before feeding to a catalytic reactor. By integrating the thermolysis and downstream catalytic conversions, producing renewable, biobased THF directly from biomass containing P4HB while at the same time eliminating costly and energy intensive unit operations is possible.

The host organism used to produce the biomass containing P4HB has been genetically modified by introduction of genes and/or deletion of genes in a wild-type or genetically engineered P4HB production organism creating strains that synthesize P4HB from inexpensive renewable feedstocks. An exemplary pathway for production of P4HB is provided in FIG. 1 and it is understood that additional enzymatic changes that contribute to this pathway can also be introduced or suppressed for a desired production of P4HB.

In one aspect, the present invention provides a process for production of a biobased tetrahydrofuran product. In certain embodiments, tetrahydrofuran in the product has 100% biobased carbon content (e.g., as determined based on ¹⁴C isotope analysis). The process includes combining a genetically engineered biomass comprising poly-4-hydroxybutyrate and a first catalyst; heating the biomass with the first catalyst converting 4-hydroxybutyrate to gamma-butyrolactone vapor. In certain embodiments, a yield of gamma-butyrolactone vapor after thermolysis is about 85% by weight or greater, for example, at least 95% based on one gram of a gamma-butyrolactone in the product per gram of the poly-4-hydroxybutyrate. The genetically engineered recombinant host produces a 4-hydroxybutyrate polymer.

In another aspect, the genetically engineered biomass for use in the processes of the invention, is from a recombinant host having a poly-4-hydroxybutyrate pathway, wherein the host has an inhibiting mutation in its CoA-independent NAD-dependent succinic semialdehyde dehydrogenase gene or its CoA-independent NADP-dependent succinic semialdehyde dehydrogenase gene, or having the inhibiting mutations in both genes, and having stably incorporated one or more genes encoding one or more enzymes selected from: a succinyl-CoA:coenzyme A transferase, a succinate semialdehyde dehydrogenase, a succinic semialdehyde reductase, a CoA transferase, and a polyhydroxyalkanoate synthase, wherein the succinyl-CoA:coenzyme A transferase converts succinate to succinyl-CoA, the succinate semialdehyde dehydrogenase converts succinyl-CoA to succinic semialdehyde, the succinic semialdehyde reductase converts succinic semialdehyde to 4-hydroxybutyrate, the CoA transferase converts 4-hydroxybutyrate to 4-hydroxybutyryl-CoA and the polyhydroxyalkanoate synthase polymerizes 4-hydroxybutyryl-CoA to poly-4-hydroxybutyrate. In a further aspect, the host has two or more, three or more, four or more or all five of the stably incorporating genes encoding the enzymes listed above.

In yet another aspect of the invention, the genetically engineered biomass for use in the processes of the invention the genetically engineered biomass is from a recombinant host having stably incorporated one or more genes encoding one or more enzymes selected from: a phosphoenolpyruvate carboxylase, an isocitrate lyase, a malate synthase, an ADP-forming succinate-CoA ligase, an NADP-dependent glyceraldeyde-3-phosphate dehydrogenase, an NAD-dependent glyceraldeyde-3-phosphate dehydrogenase, a butyrate kinase, and a phosphotransbutyrylase; and optionally having a disruption in one or more genes selected from yneI, gabD, pykF, pykA, maeA and maeB, wherein the phosphoenolpyruvate carboxylase converts phosphoenolpyruvate to oxaloacetate, the isocitrate lyase converts isocitrate to glyoxalate, the malate synthase converts glyoxalate to malate and succinate, the ADP-forming succinate-CoA ligase converts succinate to succinyl-CoA, the NADP-dependent glyceraldeyde-3-phosphate dehydrogenase converts glyceraldehyde 3-phosphate to 1,3-bisphosphoglycerate forming NADPH+H⁺, the NAD-dependent glyceraldeyde-3-phosphate dehydrogenase converts glyceraldehyde 3-phosphate to 1,3-bisphosphoglycerate forming NADH+H⁺, the butyrate kinase converts 4-hydroxybutyrate to 4-hydroxybutyryl-phosphate, and the phosphotransbutyrylase converts 4-hydroxybutyryl-phosphate to 4-hydroxybutyryl-CoA.

In a further aspect, the genetically engineered biomass for use in the processes of the invention is from a recombinant host having a poly-4-hydroxybutyrate pathway and stably expressing two or more genes encoding two or more enzymes, three or more genes encoding three or more enzymes, four of more genes encoding four or more enzymes or five or more genes encoding five or more enzymes selected from: a phosphoenolpyruvate carboxylase, an isocitrate lyase, a malate synthase, an ADP-forming succinate-CoA ligase, an NADP-dependent glyceraldeyde-3-phosphate dehydrogenase, an NAD-dependent glyceraldeyde-3-phosphate dehydrogenase, a butyrate kinase, and a phosphotransbutyrylase; and optionally having a disruption in one or more genes selected from yneI, gabD, pykF, pykA, maeA and maeB, wherein the phosphoenolpyruvate carboxylase converts phosphoenolpyruvate to oxaloacetate, the isocitrate lyase converts isocitrate to glyoxalate, the malate synthase converts glyoxalate to malate and succinate, the ADP-forming succinate-CoA ligase converts succinate to succinyl-CoA, the NADP-dependent glyceraldeyde-3-phosphate dehydrogenase converts glyceraldehyde 3-phosphate to 1,3-bisphosphoglycerate forming NADPH+H⁺, the NAD-dependent glyceraldeyde-3-phosphate dehydrogenase converts glyceraldehyde 3-phosphate to 1,3-bisphosphoglycerate forming NADH+H⁺, the butyrate kinase converts 4-hydroxybutyrate to 4-hydroxybutyryl-phosphate, and the phosphotransbutyrylase converts 4-hydroxybutyryl-phosphate to 4-hydroxybutyryl-CoA.

In another embodiment, the genetically engineered biomass for use in the processes of the invention is from a recombinant host having a poly-4-hydroxybutyrate pathway, wherein the host has an inhibiting mutation in its CoA-independent NAD-dependent succinic semialdehyde dehydrogenase gene or its CoA-independent NADP-dependent succinic semialdehyde dehydrogenase gene, or having inhibiting mutations in both genes, and having stably incorporated genes encoding the following enzymes: a succinyl-CoA:coenzyme A transferase wherein the succinyl-CoA:coenzyme A transferase is able to convert succinate to succinyl-CoA, a succinate semialdehyde dehydrogenase wherein the succinate semialdehyde dehydrogenase is able to convert succinyl-CoA to succinic semialdehyde, a succinic semialdehyde reductase wherein the succinic semialdehyde reductase is able to convert succinic semialdehyde to 4-hydroxybutyrate, a CoA transferase wherein the CoA transferase is able to convert 4-hydroxybutyrate to 4-hydroxybutyryl-CoA, and a polyhydroxyalkanoate synthase wherein the polyhydroxyalkanoate synthase is able to polymerize 4-hydroxybutyryl-CoA to poly-4-hydroxybutyrate.

In yet another embodiment, In certain processes of the invention, the genetically engineered biomass is from a recombinant host having a poly-4-hydroxybutyrate pathway, wherein the host has optionally an inhibiting mutation in its CoA-independent NAD-dependent succinic semialdehyde dehydrogenase gene or its CoA-independent NADP-dependent succinic semialdehyde dehydrogenase gene, or having inhibiting mutations in both genes, and having stably incorporated genes encoding the following enzymes: a succinyl-CoA:coenzyme A transferase, a succinate semialdehyde dehydrogenase, a succinic semialdehyde reductase, a CoA transferase, and a polyhydroxyalkanoate synthase, wherein the succinyl-CoA:coenzyme A transferase converts succinate to succinyl-CoA, the succinate semialdehyde dehydrogenase converts succinyl-CoA to succinic semialdehyde, the succinic semialdehyde reductase converst succinic semialdehyde to 4-hydroxybutyrate, the CoA transferase converts 4-hydroxybutyrate to 4-hydroxybutyryl-CoA, and the polyhydroxyalkanoate synthase polymerizes 4-hydroxybutyryl-CoA to poly-4-hydroxybutyrate.

In other embodiments, the genetically engineered biomass used in the processes of the invention is from a recombinant host having stably incorporated genes encoding the following enzymes: a phosphoenolpyruvate carboxylase, an isocitrate lyase, a malate synthase wherein the malate synthase is able to convert glyoxalate to malate and succinate, an ADP-forming succinate-CoA ligase, an NADP-dependent glyceraldeyde-3-phosphate dehydrogenase wherein the NADP-dependent glyceraldeyde-3-phosphate dehydrogenase is able to convert glyceraldehyde 3-phosphate to 1,3-bisphosphoglycerate forming NADPH+H⁺, an NAD-dependent glyceraldeyde-3-phosphate dehydrogenase, a butyrate kinase, a phosphotransbutyrylase; and optionally having a disruption in one or more genes selected from yneI, gabD, pykF, pykA, maeA and maeB, wherein the phosphoenolpyruvate carboxylase converts phosphoenolpyruvate to oxaloacetate, the isocitrate lyase converts isocitrate to glyoxalate, the succinate-CoA ligase (ADP-forming) converts succinate to succinyl-CoA, the NAD-dependent glyceraldeyde-3-phosphate dehydrogenase converts glyceraldehyde 3-phosphate to 1,3-bisphosphoglycerate forming NADH+H⁺, the butyrate kinase converts 4-hydroxybutyrate to 4-hydroxybutyryl-phosphate and the phosphotransbutyrylase converts 4-hydroxybutyryl-phosphate to 4-hydroxybutyryl-CoA.

In other methods of the invention, the genetically engineered biomass is from a recombinant host having a poly-4-hydroxybutyrate pathway, wherein the host has stably incorporated one or more genes encoding one or more enzymes selected from a succinyl-CoA:coenzyme A transferase, a succinate semialdehyde dehydrogenase, a succinic semialdehyde reductase, a CoA transferase, and a polyhydroxyalkanoate synthase wherein the succinyl-CoA:coenzyme A transferase converts succinate to succinyl-CoA, the succinate semialdehyde dehydrogenase converts succinyl-CoA to succinic semialdehyde, the succinic semialdehyde reductase converts succinic semialdehyde to 4-hydroxybutyrate, the CoA transferase converts 4-hydroxybutyrate to 4-hydroxybutyryl-CoA, and the polyhydroxyalkanoate synthase polymerizes 4-hydroxybutyryl-CoA to poly-4-hydroxybutyrate.

In still other embodiments of the processes of the invention, the genetically engineered biomass is from a recombinant host having stably incorporated one or more genes encoding one or more enzymes selected from: a phosphoenolpyruvate carboxylase, an isocitrate lyase, a malate synthase, an ADP-forming succinate-CoA ligase, an NADP-dependent glyceraldeyde-3-phosphate dehydrogenase, an NAD-dependent glyceraldeyde-3-phosphate dehydrogenase, a butyrate kinase, a phosphotransbutyrylase; and optionally having a disruption in one or more genes selected from yneI, gabD, pykF, pykA, maeA and maeB, wherein the phosphoenolpyruvate carboxylase converts phosphoenolpyruvate to oxaloacetate, the isocitrate lyase converts isocitrate to glyoxalate, the malate synthase converts glyoxalate to malate and succinate, the ADP-forming succinate-CoA ligase converts succinate to succinyl-CoA, the NADP-dependent glyceraldeyde-3-phosphate dehydrogenase converts glyceraldehyde 3-phosphate to 1,3-bisphosphoglycerate forming NADPH+H⁺, the NAD-dependent glyceraldeyde-3-phosphate dehydrogenase converts glyceraldehyde 3-phosphate to 1,3-bisphosphoglycerate forming NADH+H⁺, the butyrate kinase converts 4-hydroxybutyrate to 4-hydroxybutyryl-phosphate, and the phosphotransbutyrylase converts 4-hydroxybutyryl-phosphate to 4-hydroxybutyryl-CoA.

In certain embodiments, the processes described herein further include an initial step of culturing a recombinant host with a renewable feedstock to produce a poly-4-hydroxybutyrate biomass.

In a certain aspect of the invention, a recombinant host is cultured with a renewable feedstock to produce a 4-hydroxybutyrate biomass, the produced biomass is then treated in the presence of a first catalyst to produce gamma-butyrolactone (GBL) vapor, wherein a yield of gamma-butyrolactone vapor is about 85% by weight, about 86% by weight, about 87% by weight, about 88% by weight, about 89% by weight, about 90% by weight, about 91% by weight, about 92% by weight, about 93% by weight, about 94% by weight, about 95% by weight, about 96% by weight, about 97% by weight, about 98% by weight, or about 99% by weight. The GBL vapor is then exposed in situ to a second catalyst and hydrogen gas converting the biobased GBL to tetrahydrofuran (THF).

In certain embodiments, the source of the renewable feedstock is selected from glucose, fructose, sucrose, arabinose, maltose lactose xylose, fatty acids, vegetable oils, and biomass derived synthesis gas or a combination thereof.

The invention also pertains to a biobased tetrahydrofuran product produced by the processes described herein. In certain aspects, the amount of gamma-butyrolactone in the vapor phase produced is 85% or greater than 85%, for example, 86% by weight, about 87% by weight, about 88% by weight, about 89% by weight, about 90% by weight, about 91% by weight, about 92% by weight, about 93% by weight, about 94% by weight, about 95% by weight, about 96% by weight, about 97% by weight, about 98% by weight, or about 99% by weight. In a further aspect, the invention pertains to a poly-4-hydroxybutyrate biomass produced from renewable resources which is suitable as a feedstock for producing tetrahydrofuran product, wherein the level of poly-4-hydroxybutyrate in the biomass is greater than 50% by weight of the biomass, for example between about 50% by weight and 90% by weight, for example greater than 55 to about 75%, or greater than 60%, to about 80%, or greater that 70% to about 90%.

In certain embodiments of the invention, the biomass host is bacteria, yeast, fungi, algae, cyanobacteria, or a mixture of any two or more thereof. The bacteria includes but is not limited to Escherichia coli, Alcaligenes eutrophus (renamed as Ralstonia eutropha), Bacillus spp., Alcaligenes latus, Azotobacter, Aeromonas, Comamonas, Pseudomonads Ralstonia, Pseudomonas Klebsiella, Synechococcus sp PCC7002, Synechococcus sp. PCC 7942, Synechocystis sp. PCC 6803, and Thermosynechococcus elongatus BP-I (cyanobacteria), Chlorobium tepidum (green sulfur bacteria), Chloroflexus auranticus (green non-sulfur bacteria), Chromatium tepidum and Chromatium vinosum (purple sulfur bacteria), Rhodospirillum rubrum, Rhodobacter capsulatus, and Rhodopseudomonas palustris. In other embodiments, the recombinant host is algae. The algae include but are not limited to Chlorella minutissima, Chlorella emersonii, Chlorella sorokiniana, Chlorella ellipsoidea, Chlorella sp., or Chlorella protothecoides.

In certain embodiments of the invention, the heating of the P4HB biomass is at a temperature of about 100° C. to about 350° C. or about 200° C. to about 350° C., or from about 225° C. to 300° C. In some embodiments, the heating reduces the water content of the biomass to about 5 wt % or less (e.g., less than 5%, less than 4%, less than 3%, less than 2%, less than 1%, less than 0.5%). In the embodiments described, the heating is for a time period from about 30 seconds to about 5 minutes or is from about 5 minutes to about 2 hours. In certain embodiments the tetrahydrofuran product comprises less than 5% of undesired side products. In certain embodiments, the first catalyst is sodium carbonate or calcium hydroxide, or mixtures thereof. The weight percent of the first catalyst is in the range of about 4% to about 50%, for example between about 10% to about 40%, for example about 20% to about 30%, for example, 5%, 6%, 7%, 8%, 9%, 10%. In particular embodiments, the weight % of the first catalyst is in the range of about 4% to about 50%, and the heating is at about 300° C. In particular embodiments, the process includes about 4% calcium carbonate as the first catalyst heated at about 300° C. In certain embodiments, the gamma-butyrolactone vapor is further processed in situ by first passing it through an absorbent bed to remove nitrogen impurities, wherein the absorbent can be activated carbon, clay, alumina, zeolite, or silica gel or any combination of these, then the GBL vapor is mixed with hydrogen, pressurized (compressed) and heated to about 190° C. and passed over a second catalyst. In preferred embodiments, the second catalyst is a mixture of zinc, aluminum, copper, chrome, cobalt and nickel oxides.

It is also contemplated that other catalysts may be used to in the processes described herein to produce tetrahydrofuran and other chemical precurers alone or in mixtures. For example, the processes as described herein may include catalyst for producing tetrahydrofuran and 1,4-butanediol (BDO) mixtures.

In certain aspects of the processes of the invention, the gamma-butyrolactone vapor comprises less than 5% by weight of side products. In certain embodiments, the gamma-butyrolactone vapor is partially condensed at a condenser temperature of greater than 200° C. prior to hydrogenation by controlled cooling to allow high boiling impurities to be substantially removed from the vapor. Other impurities can also be selectively separated from GBL, for example, the partially condensed gamma-butyrolactone vapor is passed through an absorbent bed containing activated carbon, clay, silica gel, alumina, or a mixture of these.

In other embodiments of the invention, the gamma-butyrolactone vapor during hydrogenating is mixed with hydrogen gas at a weight ratio of hydrogen gas/GBL of 99%/1% to about 95%/5%. In these embodiments, the hydrogen/GBL gas mixture is compressed for example from 2 to about 8 bars and can be heated to 180° C. to about 210° C.

In certain embodiments, the second catalyst is a vapor phase hydrogenation catalyst, for example a mixture of about 20 to about 40% CuO, about 20 to about 40% ZnO, about 5 to about 20% Al₂O₃ by weight. In certain processes described herein, the heated and compressed, hydrogen/GBL gas mixture is exposed to the second catalyst.

Additionally, the expended (residual) PHA reduced biomass is further utilized for energy development, for example as a fuel to generate process steam and/or heat.

A poly-4-hydroxybutyrate biomass produced from renewable resources which is suitable as a feedstock for producing gamma-butyrolactone product is obtained from the processes described herein. The level of poly-4-hydroxybutyrate in the biomass is greater than 50% by weight of the biomass. The biobased tetrahydrofuran product in certain aspects has 100% biobased carbon content. Further, the product yield from the processes described herein is about 95% by weight or greater based on one gram of a tetrahydrofuran in the product per gram of gamma butyrlactone vapor.

In certain aspects, the processes described herein produce a tetrahydrofuran product comprises less than 5% by weight of side products, for example, less than 4% by weight, less than 3% by weight, less than 2% by weight, less than 1% by weight, less than 0.5% by weight.

The resultant tetrahydrofuran product may be further processed into other chemicals. For example, the polytetrahydrofuran is an intermediate for elastic fibers such as spandex (e.g., elastane, lycra, elaspan, creora ROICA, dorlastan, linel and ESPA) and for polyurethane resins. These elastomers are either polyurethanes made by reacting the polytetrahydrofuran with diisocyanates, or polyesters made by reacting polytetrahydrofuran with diacids or their derivatives. The polytetrahydrofurn can be prepared by acid catalyzed polymerization of tetrahydrofuran.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram of exemplary E. coli central metabolic pathways showing reactions that were modified or introduced in the Examples or could be modified. Numbers in the figure refer to reaction numbers in Table 1A. Reactions that were eliminated by deleting the corresponding genes are marked with an “X”. Abbreviations: “GA3P”, D-glyceraldehyde-3-phosphate; “G1,3P”, 1,3-diphosphateglycerate; “PEP”, phosphoenolpyruvate; “PYR”, pyruvate; “AcCoA”, acetyl-CoA; “CIT”, citrate; “ICT”, isocitrate; “αKG”, alpha-ketoglutarate; “SUC-CoA”, succinyl CoA; “SUC”, succinate; “Fum”, fumarate; “MAL”, L-malate; “OAA”, oxaloacetate; “SSA”, succinic semialdehyde; “4HB”, 4-hydroxybutyrate; “4HB-CoA”, 4-hydroxybutyryl CoA; “P4HB”, poly-4-hydroxybutyrate. Numbered reactions: “1”, glyceraldehyde-3-phosphate dehydrogenase; “2”, pyruvate kinase; “3”, phosphoenolpyruvate carboxylase; “4”, malic enzyme; “5”, isocitrate lyase; “6”, malate dehydrogenase; “7”, succinate semialdehyde dehydrogenase; “8”, alpha-ketoglutarate decarboxylase; “9”, succinic semialdehyde reductase; “10”, CoA transferase; “11”, polyhydroxyalkanoate synthase; “12”, succinate-semialdehyde dehydrogenase, NADP+-dependent.

FIG. 2 is a schematic of GBL recovery from biomass with residual converted to solid fuel, according to various embodiments.

FIG. 3 is a weight loss vs. time curve at 300° C. in N₂ for dry P4HB fermentation broth without lime (solid curve) and with 5% lime addition (dashed curve), according to various embodiments. The curves show the weight loss slopes and onset times for completed weight loss.

FIG. 4 (A-C) is a series of gas chromatograms of P4HB pure polymer, P4HB dry broth and P4HB dry broth+5% lime (Ca(OH)₂) catalyst after pyrolysis at 300° C., according to one embodiment.

FIG. 5 is a mass spectral library match of GC-MS peak @6.2 min to GBL (gamma-butyrolactone) according to one embodiment.

FIG. 6 is a mass spectral library match of GC-MS peak @11.1 min peak for GBL dimer according to one embodiment.

FIG. 7 is a schematic diagram of the equipment used for the scaled up pyrolysis of P4HB biomass.

FIG. 8 is a schematic diagram of the direct conversion of P4HB biomass to biobased THF via catalytic vapor phase hydrogenation.

### DETAILED DESCRIPTION OF THE INVENTION

A description of example embodiments of the invention follows.

The present invention provides processes and methods for the manufacture of biobased tetrahydrofuran (THF) from a genetically engineered microbe producing poly-4-hydroxybutyrate polymer (P4HB biomass). For the purposes of this invention P4HB is defined to also include the copolymer of 4-hydroxybutyrate with 3-hydroxybutyrate where the % of 4-hydroxybutyrate in the copolymer is greater than 80%, 85%, 90% preferably greater than 95% of the monomers in the copolymer. In certain embodiments, the P4HB biomass is produced by improved P4HB production processes using the recombinant hosts described herein. These recombinant hosts have been genetically constructed to increase the yield of P4HB by manipulating (e.g., inhibition and/or overexpression) certain genes in the P4HB pathway to increase the yield of P4HB in the biomass. The P4HB biomass is produced in a fermentation process in which the genetically engineered microbe is fed a renewable substrate. Renewable substrates include fermentation feedstocks such as sugars, vegetable oils, fatty acids or synthesis gas produced from plant crop materials. The level of P4HB produced in the biomass from the sugar substrate is greater than 10% (e.g., about 20%, about 30%, about 40%, about 50%, about 60%, about 70%, about 80%) of the total dry weight of the biomass. The P4HB biomass is then combined with a first catalyst and heated to thermally decompose the P4HB to biobased GBL vapor, after the GBL vapor is mixed with hydrogen gas and exposed to a second catalyst which converts the GBL to tetrahydrofuran (THF).

Described herein are an alternative processes for manufacturing THF based on using renewable carbon sources to produce a biobased poly-4-hydroxybutyrate (P4HB) polymer in a biomass that is then converted to biobased tetrahydrofuran product.

Biobased, biodegradable polymers such as polyhydroxyalkanoates (PHAs), are naturally produced in biomass systems, such as microbial biomass (e.g., bacteria including cyanobacteria, yeast, fungi), plant biomass, or algal biomass. Genetically-modified biomass systems have been developed which produce a wide variety of biodegradable PHA polymers and copolymers in high yield (Lee (1996), Biotechnology & Bioengineering 49:1-14; Braunegg et al. (1998), J. Biotechnology 65:127-161; Madison, L. L. and Huisman, G. W. (1999), Metabolic Engineering of Poly-3-Hydroxyalkanoates; From DNA to Plastic, in: Microbiol. Mol. Biol. Rev. 63:21-53). PHA polymers are well known to be thermally unstable compounds that readily degrade when heated up to and beyond their melting points (Cornelissen et al., Fuel, 87, 2523, 2008). This is usually a limiting factor when processing the polymers for plastic applications that can, however, be leveraged to create biobased, chemical manufacturing processes starting from 100% renewable resources.

When pure poly-4-hydroxybutyrate (P4HB), produced using petroleum derived 1,4-butanediol, is heated up to 250-350° C., it thermally degrades to volatile GBL exclusively by unzipping of the polymer chain (Kim et al. (2006), Polymer Degradation and Stability, 91:2333-2341). As described herein, the addition of low cost catalysts to a genetically engineered biomass with an increased production of P4HB from glucose speeds up the degradation reaction to gamma-butyrolactone vapor. The inexpensive catalyst is left with the residual biomass or can optionally be recycled back to the process after suitable regeneration including thermal regeneration. Combining the first catalysis reaction with specifically genetically modified, high yielding P4HB producing biomass is an economical and environmental alternative to the traditional petroleum-based processes for producing GBL. The GBL so produced can then be further processed while still in the vapor state (in situ) by mixing it with hydrogen, pressurizing and heating the mixer and then passing it over a second catalyst to hydrogenate the GBL to THF. This greatly improves the energetics of the process as it avoids the energy intensive operations of condensing GBL and subsequently vaporizing the same GBL before feeding to a catalytic reactor. By integrating the thermolysis and downstream catalytic conversions, it is now possible to produce renewable, biobased THF directly from biomass containing P4HB while at the same time eliminating costly and energy intensive unit operations.

Recombinant Hosts with Metabolic Pathways for Producing P4HB

Genetic engineering of hosts (e.g., bacteria, fungi, algae, plants and the like) as production platforms for modified and new materials provides a sustainable solution for high value eco-friendly industrial applications for production of chemicals. Described herein are process methods of producing biobased gamma-butyrolactone from a genetically modified recombinant polyhydroxyalkanoate P4HB biomass. The processes described herein avoid toxic effects to the host organism by producing the biobased chemical post culture or post harvesting, are cost effective and highly efficient (e.g., use less energy to make), decrease greenhouse gas emissions, use renewable resources and can be further processed to produce high purity products like tetrahydrofuran from GBL in high yield.

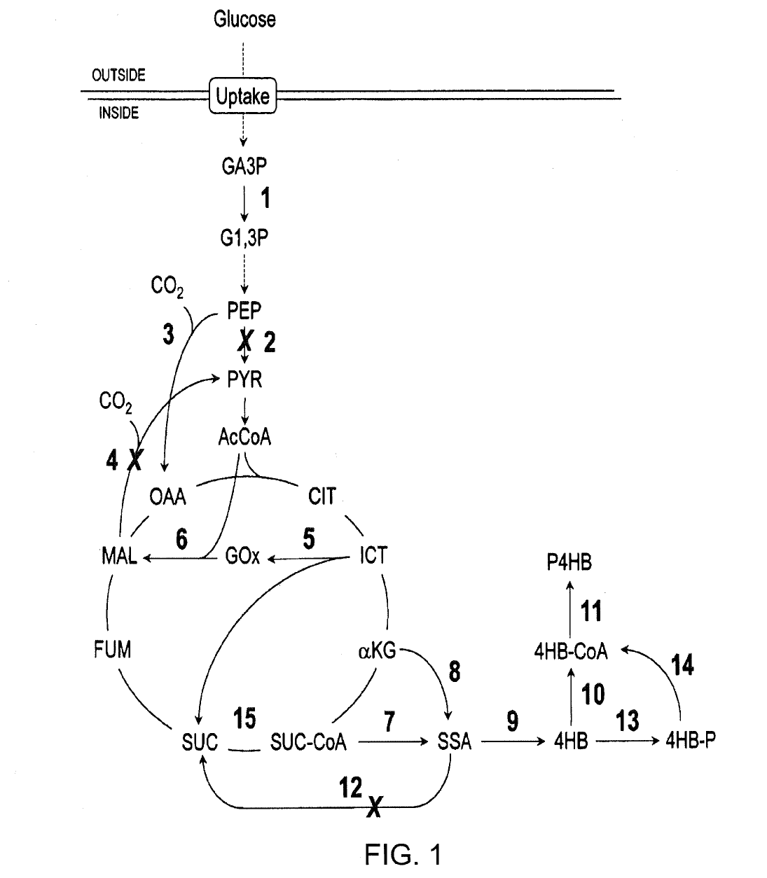

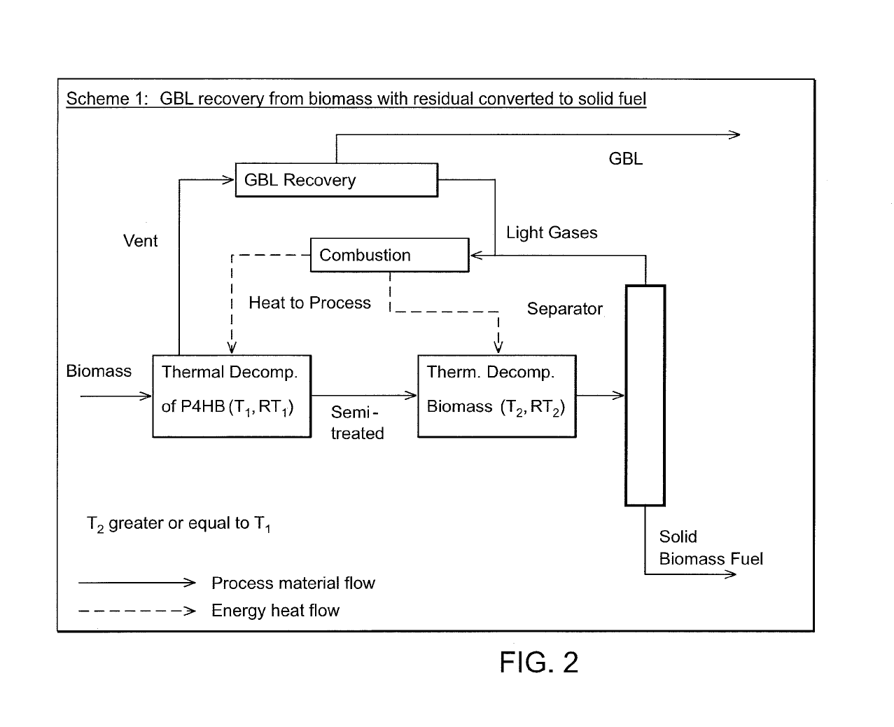

The PHA biomass utilized in the methods described herein is genetically engineered to produce poly-4-hydroxybutyrate (P4HB). An exemplary pathway for production of P4HB is provided in FIG. 1 and a more detailed description of the pathway, recombinant hosts that produce P4HB biomass is provided below. The pathway can be engineered to increase production of P4HB from carbon feed sources.

As used herein, “P4HB biomass” is intended to mean any genetically engineered biomass from a recombinant host (e.g., bacteria,) that includes a non-naturally occurring amount of the polyhydroxyalkanoate polymer e.g. poly-4-hydroxybutyrate (P4HB). In some embodiments, a source of the P4HB biomass is bacteria, yeast, fungi, algae, plant crop cyanobacteria, or a mixture of any two or more thereof. In certain embodiments, the biomass titer (g/L) of P4HB has been increased when compared to the host without the overexpression or inhibition of one or more genes in the P4HB pathway. In certain embodiments, the P4HB titer is reported as a percent dry cell weight (% dcw) or as grams of P4HB/Kg biomass.

“Overexpression” refers to the expression of a polypeptide or protein encoded by a DNA introduced into a host cell, wherein the polypeptide or protein is either not normally present in the host cell, or where the polypeptide or protein is present in the host cell at a higher level than that normally expressed from the endogenous gene encoding the polypeptide or protein. “Inhibition” or “down regulation” refers to the suppression or deletion of a gene that encodes a polypeptide or protein. In some embodiments, inhibition means inactivating the gene that produces an enzyme in the pathway. In certain embodiments, the genes introduced are from a heterologous organism.

Genetically engineered microbial PHA production systems with fast growing hosts such as Escherichia coli have been developed. In certain embodiments, genetic engineering also allows for the modification of wild-type microbes to improve the production of the P4HB polymer. Examples of PHA production modifications are described in Steinbuchel & Valentin, FEMS Microbiol. Lett. 128:219-28 (1995). PCT Publication No. WO 98/04713 describes methods for controlling the molecular weight using genetic engineering to control the level of the PHA synthase enzyme. Commercially useful strains, including Alcaligenes eutrophus (renamed as Ralstonia eutropha), Alcaligenes latus, Azotobacter vinlandii, and Pseudomonads, for producing PHAs are disclosed in Lee, Biotechnology & Bioengineering, 49:1-14 (1996) and Braunegg et al., (1998), J. Biotechnology 65: 127-161. U.S. Pat. Nos. 6,316,262, 7,229,804 6,759,219 and 6,689,589 describe biological systems for manufacture of PHA polymers containing 4-hydroxyacids, incorporated by reference herein.

Although there have been reports of producing 4-hydroxybutyrate copolymers from renewable resources such as sugar or amino acids, the level of 4HB in the copolymers produced from scalable renewable substrates has been much less than 50% of the monomers in the copolymers and therefore unsuitable for practicing the disclosed invention. Production of the P4HB biomass using an engineered microorganism with renewable resources where the level of P4HB in the biomass is sufficient to practice the disclosed invention (i.e., greater than 40%, 50%, 60% or 65% of the total biomass dry weight) has not previously been achieved.

The weight percent PHA in the wild-type biomass varies with respect to the source of the biomass. For microbial systems produced by a fermentation process from renewable resource-based feedstocks such as sugars, vegetable oils or glycerol, the amount of PHA in the wild-type biomass may be about 65 wt %, or more, of the total weight of the biomass. For plant crop systems, in particular biomass crops such as sugarcane or switchgrass, the amount of PHA may be about 3%, or more, of the total weight of the biomass. For algae or cyanobacterial systems, the amount of PHA may be about 40%, or more of the total weight of the biomass.

In certain aspects of the invention, the recombinant host has been genetically engineered to produce an increased amount of P4HB as compared to the wild-type host. The wild-type P4HB biomass refers to the amount of P4HB that an organism typically produces in nature.

For example, in certain embodiments, the P4HB is increased between about 20% to about 90% over the wild-type or between about 50% to about 80%. In other embodiments, the recombinant host produces at least about a 20% increase of P4HB over wild-type, at least about a 30% increase over wild-type, at least about a 40% increase over wild-type, at least about a 50% increase over wild-type, at least about a 60% increase over wild-type, at least about a 70% increase over wild-type, at least about a 75% increase over wild-type, at least about a 80% increase over wild-type or at least about a 90% increase over wild-type. In other embodiments, the P4HB is between about a 2 fold increase to about a 400 fold increase over the amount produced by the wild-type host. The amount of P4HB in the host or plant is determined by gas chromatography according to procedures described in Doi, Microbial Polyesters, John Wiley&Sons, p24, 1990. In certain embodiments, a biomass titer of 100-120 g P4HB/Kg of biomass is achieved. In other embodiments, the amount of P4HB titer is presented as percent dry cell weight (% dcw).

### Suitable Host Strains

In certain embodiments, the host strain is E. coli K-12 strain LS5218 (Spratt et al., J. Bacteriol. 146 (3):1166-1169 (1981); Jenkins and Nunn, J. Bacteriol. 169 (1):42-52 (1987)). Other suitable E. coli K-12 host strains include, but are not limited to, MG1655 (Guyer et al., Cold Spr. Harb. Symp. Quant. Biol. 45:135-140 (1981)), WG1 and W3110 (Bachmann Bacteriol. Rev. 36(4):525-57 (1972)). Alternatively, E. coli strain W (Archer et al., BMC Genomics 2011, 12:9 doi:10.1186/1471-2164-12-9) or E. coli strain B (Delbruck and Luria, Arch. Biochem. 1:111-141 (1946)) and their derivatives such as REL606 (Lenski et al., Am. Nat. 138:1315-1341 (1991)) are other suitable E. coli host strains.

Other exemplary microbial host strains include but are not limited to: Ralstonia eutropha, Zoogloea ramigera, Allochromatium vinosum, Rhodococcus ruber, Delftia acidovorans, Aeromonas caviae, Synechocystis sp. PCC 6803, Synechococcus elongatus PCC 7942, Thiocapsa pfenigii, Bacillus megaterium, Acinetobacter baumannii, Acinetobacter baylyi, Clostridium kluyveri, Methylobacterium extorquens, Nocardia corralina, Nocardia salmonicolor, Pseudomonas fluorescens, Pseudomonas oleovorans, Pseudomonas sp. 6-19, Pseudomonas sp. 61-3 and Pseudomonas putida, Rhodobacter sphaeroides, Alcaligenes latus, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, and Clostridium acetobutylicum. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris.

Exemplary algal strains species include but are not limited to: Chlorella strains, species selected from: Chlorella minutissima, Chlorella emersonii, Chlorella sorokiniana, Chlorella ellipsoidea, Chlorella sp., or Chlorella protothecoides.

### Source of Recombinant Genes

Sources of encoding nucleic acids for a P4HB pathway enzyme can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Saccharomyces cerevisiae, Saccharomyces kluyveri, Clostridium kluyveri, Clostridium acetobutylicum, Clostridium beijerinckii, Clostridium saccharoperbutylacetonicum, Clostridium perjringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Chlorella minutissima, Chlorella emersonii, Chlorella sorokiniana, Chlorella ellipsoidea, Chlorella sp., Chlorella protothecoides, Homo sapiens, Oryctolagus cuniculus, Rhodobacter spaeroides, Thermoanaerobacter brockii, Metallosphaera sedula, Leuconostoc mesenteroides, ChloroJlexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salina rum, Geobacillus stearothermophilus, Aeropyrum pernix, Sus scrofa, Caenorhabditis elegans, Corynebacterium glutamicum, Acidaminococcusfermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum marine gamma proteobacterium, and butyrate-producing bacterium. For example, microbial hosts (e.g., organisms) having P4HB biosynthetic production are exemplified herein with reference to an E. coli host. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite P4HB biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of P4HB and other compounds of the invention described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

### Production of Transgenic Host for Producing 4HB

Transgenic (Recombinant) hosts for producing P4HB are genetically engineered using conventional techniques known in the art. The genes cloned and/or assessed for host strains producing P4HB-containing PHA and 4-carbon chemicals are presented below in Table 1A, along with the appropriate Enzyme Commission number (EC number) and references. Some genes were synthesized for codon optimization while others were cloned via PCR from the genomic DNA of the native or wild-type host. As used herein, “heterologous” means from another host. The host can be the same or different species. FIG. 1 is an exemplary pathway for producing P4HB.

TABLE 1A
Genes in microbial host strains producing 4HB-containing PHA and 4-carbon
chemicals. A star (*) after the gene name denotes that the nucleotide sequence was
optimized for expression in E. coli.

| Reaction | | | | |
|---|---|---|---|---|
| number | | | | |
| (FIG. 1) | Gene Name | Enzyme Name | EC Number | Accession No. |
| 1 | gapA | Glyceraldehyde 3- | 1.2.1.12 | NP_416293 |
| | | phosphate dehydrogenase | | |
| 1 | gdp1 | Glyceraldehyde-3- | 1.2.1.12 | XP_455496 |
| | | phosphate dehydrogenase | | |
| 1 | gap2 | Glyceraldehyde-3- | 1.2.1.59 | CAA58550 |
| | | phosphate dehydrogenase | | |
| | | (NADP+) | | |
| | | (phosphorylating) | | |
| 1 | gapB | Glyceraldehyde-3- | 1.2.1.59 | NP_390780 |
| | | phosphate dehydrogenase 2 | | |
| 1 | gapN | Putative NADP- | 1.2.1.12 | NP_664849 |
| | | dependent | | |
| | | glyceraldehyde-3- | | |
| | | phosphate dehydrogenase | | |
| 2 | pykF | Pyruvate kinase I | 2.7.1.40 | b1676 |
| 2 | pykA | Pyruvate kinase II | 2.7.1.40 | b1854 |
| 3 | ppc_{Ec} | Phosphoenolpyruvate | 4.1.1.31 | NP_418391 |
| | | carboxylase | | |
| 3 | ppc_{Ms}* | Phosphoenolpyruvate | 4.1.1.31 | Gene/Protein ID 1; |
| | | carboxylase | | Q02735 |
| 4 | maeA | Malate dehydrogenase, | 1.1.1.38 | b1479 |
| | | NAD-requiring | | |
| 4 | maeB | Malate dehydrogenase | 1.1.1.40 | b2463 |
| | | (oxaloacetate- | | |
| | | decarboxylating) | | |
| | | (NADP+) | | |
| 5 | aceA | Isocitrate lyase | 4.1.3.1 | NP_418439 |
| 6 | aceB | Malate synthase A | 2.3.3.9 | NP_418438 |
| 7 | sucD* | Succinate semialdehyde | 1.2.1.76 | Gene/Protein ID 2; |
| | | dehydrogenase | | YP_001396394 |
| 8 | kgdM | Alpha-ketoglutarate | 4.1.1.71 | NP_335730 |
| | | decarboxylase | | |
| 9 | ssaR_{At}* | Succinic semialdehyde | 1.1.1.61 | Gene/Protein ID 3; |
| | | reductase | | AAK94781 |
| 9 | 4hbD | Succinic semialdehyde | 1.1.1.61 | YP_001396393 |
| | | reductase | | |
| 9 | ssaR_{At2}* | Succinic semialdehyde | 1.1.1.61 | Gene/Protein ID 4; |
| | | reductase | | XP_001210625 |
| 9 | ssaR_{Mm}* | Succinic semialdehyde | 1.1.1.61 | Gene/Protein ID 5; |
| | | reductase | | AKR7A5; |
| 9 | yqhD | Succinic semialdehyde | 1.1.1.61 | NP_417484 |
| | | reductase | | |
| 10 | orfZ | CoA transferase | 2.8.3.n | AAA92344 |
| 11 | phaC1 | Polyhydroxyalkanoate | 2.3.1.n | YP_725940 |
| | | synthase | | |
| 11 | phaC3/C1* | Polyhydroxyalkanoate | 2.3.1.n | Gene/Protein ID 6 |
| | | synthase fusion protein | | |
| 12 | yneI | Succinate-semialdehyde | 1.2.1.24 | NP_416042 |
| | | dehydrogenase, NADP+- | | |
| | | dependent | | |
| 12 | gabD | Succinate-semialdehyde | 1.2.1.16 | NP_417147 |
| | | dehydrogenase, NADP+- | | |
| | | dependent | | |
| 13 | buk1 | Butyrate kinase I | 2.7.2.7 | NP_349675 |
| 13 | buk2 | Butyrate kinase II | 2.7.2.7 | NP_348286 |
| 14 | ptb | Phosphotransbutyrylase | 2.3.1.19 | NP_349676 |
| 15 | sucCD | Succinate-CoA ligase | 6.2.1.5 | NP_286444 |
| | | (ADP-forming) | | NP_286445 |
| 15 | cat1 | Succinyl-CoA:coenzyme | 2.8.3.n | YP_001396395 |
| | | A transferase | | |

Other proteins capable of catalyzing the reactions listed in Table 1A can be discovered by consulting the scientific literature, patents or by BLAST searches against e.g. nucleotide or protein databases at NCBI (www.ncbi.nlm.nih.gov/). Synthetic genes can then be created to provide an easy path from sequence databases to physical DNA. Such synthetic genes are designed and fabricated from the ground up, using codons to enhance heterologous protein expression, optimizing characteristics needed for the expression system and host. Companies such as e.g. DNA 2.0 (Menlo Park, Calif. 94025, USA) will provide such routine service. Proteins that may catalyze some of the biochemical reactions listed in Table 1A are provided in Tables 1B-1Z.

TABLE 1B
Suitable homologues for the GapA protein (glyceraldehyde 3-phosphate
dehydrogenase-A, from Escherichia coli, EC No. 1.2.1.12,
which acts on D-glyceraldehyde 3-phosphate to produce
1,3-diphosphateglycerate; protein acc. no. NP_416293.1)

| Protein Name | Protein Accession No. |
|---|---|
| glyceraldehyde-3-phosphate dehydrogenase | NP_456222 |
| glyceraldehyde-3-phosphate dehydrogenase A | ZP_04561688 |
| glyceraldehyde-3-phosphate dehydrogenase | CBK85249 |
| glyceraldehyde-3-phosphate dehydrogenase, | ZP_05729429 |
| type I | |
| glyceraldehyde-3-phosphate dehydrogenase | ZP_04613128 |
| glyceraldehyde-3-phosphate dehydrogenase | NP_929794 |
| glyceraldehyde-3-phosphate dehydrogenase A | YP_002648641 |
| glyceraldehyde-3-phosphate dehydrogenase A | CBA72924 |
| glyceraldehyde-3-phosphate dehydrogenase A | ZP_07394569 |

TABLE 1C
Suitable homologues for the Gdp1 protein (glyceraldehyde 3-phosphate
dehydrogenase, from Kluyveromyces lactis, EC No. 1.2.1.12,
which acts on D-glyceraldehyde 3-phosphate to produce
1,3-diphosphateglycerate; protein acc. no. XP_455496)

| Protein Name | Protein Accession No. |
|---|---|
| hypothetical protein | XP_446770 |
| unnamed protein product | CAA24607 |
| glyceraldehyde 3-phosphate dehydrogenase | EDN63283 |
| glyceraldehyde 3-phosphate dehydrogenase | Q9UVC0 |
| glyceraldehyde 3-phosphate dehydrogenase | XP_002171328 |
| glyceraldehyde 3-phosphate dehydrogenase | Q01077 |
| hypothetical protein CRE_18959 | XP_003115497 |
| glyceraldehyde 3-phosphate dehydrogenase | CAA06030 |
| glyceraldehyde 3-phosphate dehydrogenase | ABQ81648 |

TABLE 1D
Suitable homologues for the Gap2 protein (glyceraldehyde-3-phosphate
dehydrogenase (NADP+) (phosphorylating), from Synechocystis sp.,
EC No. 1.2.1.59, which acts on D-glyceraldehyde 3-phosphate to produce
1,3-diphosphateglycerate; protein acc. no. CAA58550)

| Protein Name | Protein Accession No. |
|---|---|
| glyceraldehyde 3-phosphate dehydrogenase | NP_442821 |
| glyceraldehyde 3-phosphate dehydrogenase | YP_003889819 |
| glyceraldehyde 3-phosphate dehydrogenase | YP_002372721 |
| unnamed protein product | CAO91151 |
| glyceraldehyde 3-phosphate dehydrogenase | ZP_01729953 |
| glyceraldehyde 3-phosphate dehydrogenase | YP_723521 |
| glyceraldehyde 3-phosphate dehydrogenase, | ZP_06309941 |
| type I | |
| glyceraldehyde 3-phosphate dehydrogenase | ZP_07113693 |
| glyceraldehyde 3-phosphate dehydrogenase | ZP_01623628 |

TABLE 1E
Suitable homologues for the GapB protein (glyceraldehyde-3-phosphate
dehydrogenase 2, from Bacillus subtilis, EC No. 1.2.1.59,
which acts on D-glyceraldehyde 3-phosphate to produce
1,3-diphosphateglycerate; protein acc. no. NP_390780)

| Protein Name | Protein Accession No. |
|---|---|
| glyceraldehyde 3-phosphate dehydrogenase | YP_003974321 |
| glyceraldehyde 3-phosphate dehydrogenase | YP_003921301 |
| glyceraldehyde 3-phosphate dehydrogenase | YP_001487767 |
| glyceraldehyde 3-phosphate dehydrogenase | YP_080196 |
| glyceraldehyde 3-phosphate dehydrogenase | YP_148579 |
| glyceraldehyde 3-phosphate dehydrogenase | YP_001376482 |
| glyceraldehyde 3-phosphate dehydrogenase | ZP_01173259 |
| glyceraldehyde 3-phosphate dehydrogenase, | ZP_06809473 |
| type I | |
| glyceraldehyde 3-phosphate dehydrogenase | YP_001126741 |

TABLE 1F
Suitable homologues for the GapN protein (putative NADP-dependent
glyceraldehyde-3-phosphate dehydrogenase, from Streptococcus pyogenes,
EC No. 1.2.1.12, which acts on D-glyceraldehyde 3-phosphate to
produce 1,3-diphosphateglycerate; protein acc. no. NP_664849)

| Protein Name | Protein Accession No. |
|---|---|
| NADP-dependent glyceraldehyde-3- | YP_002997128 |
| phosphate dehydrogenase | |
| NADP-dependent glyceraldehyde-3- | YP_002744716 |
| phosphate dehydrogenase | |
| NADP-dependent glyceraldehyde-3- | Q3C1A6 |
| phosphate dehydrogenase | |
| glyceraldehyde-3-phosphate | ZP_07725052 |
| dehydrogenase (NADP+) | |
| NADP-dependent glyceraldehyde-3- | YP_820625 |
| phosphate dehydrogenase | |
| NADP-dependent glyceraldehyde-3- | YP_001034755 |
| phosphate dehydrogenase, putative | |
| NAD-dependent DNA ligase LigA | ZP_01825832 |
| glyceraldehyde-3-phosphate | ZP_06011937 |
| dehydrogenase (NADP+) | |
| aldehyde dehydrogenase | YP_003307897 |

TABLE 1G
Suitable homologues for the Ppc protein (phosphoenolpyruvate
carboxylase, from Escherichia coli, EC No. 4.1.1.31,
which acts on phosphoenolpyruvate and carbon
dioxide to produce oxaloacetate; protein acc. no. NP_418391)

| Protein Name | Protein Accession No. |
|---|---|
| phosphoenolpyruvate carboxylase | ZP_02904134 |
| phosphoenolpyruvate carboxylase | YP_002384844 |
| phosphoenolpyruvate carboxylase | YP_003367228 |
| phosphoenolpyruvate carboxylase | ZP_02345134 |
| phosphoenolpyruvate carboxylase | ZP_04558550 |
| phosphoenolpyruvate carboxylase | YP_003615503 |
| phosphoenolpyruvate carboxylase | YP_002241183 |
| phosphoenolpyruvate carboxylase | CBK84190 |
| phosphoenolpyruvate carboxylase | YP_003208553 |

TABLE 1H
Suitable homologues for the Ppc protein (phosphoenolpyruvate
carboxylase, from Medicago sativa, EC No. 4.1.1.31,
which acts on phosphoenolpyruvate and carbon
dioxide to produce oxaloacetate; protein acc. no. Q02909)

| Protein Name | Protein Accession No. |
|---|---|
| phosphoenolpyruvate carboxylase | CAA09588 |
| phosphoenolpyruvate carboxylase | P51061 |
| phosphoenolpyruvate carboxylase 3 | AAU07998 |
| phosphoenolpyruvate carboxylase | ACN32213 |
| phosphoenolpyruvate carboxylase | BAC20365 |
| predicted protein | XP_002330719 |
| phosphoenolpyruvate carboxylase | ABV80356 |
| phosphoenolpyruvate carboxylase | AAD31452 |
| phosphoenolpyruvate carboxylase | CAJ86550 |

TABLE 1I
Suitable homologues for the AceA protein (isocitrate lyase, from
Escherichia coli K-12, EC No. 4.1.3.1, which acts on isocitrate
to produce glyoxylate and succinate; protein acc. no. NP_418439)

| Protein Name | Protein Accession No. |
|---|---|
| isocitrate lyase | NP_290642 |
| isocitrate lyase | ZP_04558565 |
| isocitrate lyase | YP_002218096 |
| isocitrate lyase, putative | YP_002932565 |
| isocitrate lyase | YP_002241049 |
| hypothetical protein ESA_00054 | YP_001436195 |
| isocitrate lyase | YP_003261295 |
| isocitrate lyase family protein | ZP_07952710 |
| isocitrate lyase | YP_002514615 |
| isocitrate lyase | YP_001234628 |

TABLE 1J
Suitable homologues for the AceB protein (malate synthase A, from
Escherichia coli K-12, EC No. 2.3.3.9, which acts on glyoxylate and
acetyl-CoA to produce malate; protein acc. no. NP_418438)

| Protein Name | Protein Accession No. |
|---|---|
| malate synthase | YP_002385083 |
| malate synthase A | ZP_06356448 |
| malate synthase | YP_002917220 |
| malate synthase | YP_001480725 |
| malate synthase | YP_001399288 |
| malate synthase A | YP_003714066 |
| malate synthase | NP_933534 |
| malate synthase A | YP_002253716 |
| malate synthase | YP_081279 |

TABLE 1K
Suitable homologues for the SucD protein (succinate semialdehyde
dehydrogenase, from Clostridium kluyveri, EC No. 1.2.1.76,
which acts on succinyl-CoA to produce succinate semialdehyde;
protein acc. no. YP_001396394)

| | Protein |
|---|---|
| Protein Name | Accession No. |
| CoA-dependent succinate semialdehyde | AAA92347 |
| dehydrogenase | |
| succinate-semialdehyde dehydrogenase | ZP_06559980 |
| [NAD(P)+] | |
| succinate-semialdehyde dehydrogenase | ZP_05401724 |
| [NAD(P)+] | |
| aldehyde-alcohol dehydrogenase family | ZP_07821123 |
| protein | |
| succinate-semialdehyde dehydrogenase | ZP_06983179 |
| [NAD(P)+] | |
| succinate-semialdehyde dehydrogenase | YP_001928839 |
| hypothetical protein CLOHYLEM_05349 | ZP_03778292 |
| succinate-semialdehyde dehydrogenase | YP_003994018 |
| [NAD(P)+] | |
| succinate-semialdehyde dehydrogenase | NP_904963 |

TABLE 1L
Suitable homologues for the KgdM protein (alpha-ketoglutarate
decarboxylase, from Mycobacterium tuberculosis,
EC No. 4.1.1.71, which acts on alpha-ketoglutarate to produce succinate
semialdehyde and carbon dioxide; protein acc. no. NP_335730)

| Protein Name | Protein Accession No. |
|---|---|
| alpha-ketoglutarate decarboxylase | YP_001282558 |
| alpha-ketoglutarate decarboxylase | NP_854934 |
| 2-oxoglutarate dehydrogenase sucA | ZP_06454135 |
| 2-oxoglutarate dehydrogenase sucA | ZP_04980193 |
| alpha-ketoglutarate decarboxylase | NP_961470 |
| alpha-ketoglutarate decarboxylase Kgd | YP_001852457 |
| alpha-ketoglutarate decarboxylase | NP_301802 |
| alpha-ketoglutarate decarboxylase | ZP_05215780 |
| alpha-ketoglutarate decarboxylase | YP_001702133 |

TABLE 1M
Suitable homologues for the SsaR_{At} protein (succinic
semialdehyde reductase, from Arabidopsis thaliana,
EC No. 1.1.1.61, which acts on succinate semialdehyde to
produce 4-hydroxybutyrate; protein acc. no. AAK94781)

| | Protein |
|---|---|
| Protein Name | Accession No. |
| 6-phosphogluconate dehydrogenase NAD- | XP_002885728 |
| binding domain-containing protein | |
| hypothetical protein isoform 1 | XP_002266252 |
| predicted protein | XP_002320548 |
| hypothetical protein isoform 2 | XP_002266296 |
| unknown | ACU22717 |
| 3-hydroxyisobutyrate dehydrogenase, | XP_002524571 |
| putative | |
| unknown | ABK22179 |
| unknown | ACJ85049 |
| predicted protein | XP_001784857 |

TABLE 1N
Suitable homologues for the 4hbD protein (succinic
semialdehyde reductase, from Clostridium kluyveri,
EC No. 1.1.1.61, which acts on succinate semialdehyde to
produce 4-hydroxybutyrate; protein acc. no. YP_001396393)

| Protein Name | Protein Accession No. |
|---|---|
| NAD-dependent 4-hydroxybutyrate | NP_348201 |
| dehydrogenase | |
| NAD-dependent 4-hydroxybutyrate | ZP_05401720 |
| dehydrogenase | |
| 4-hydroxybutyrate dehydrogenase | ZP_06902666 |
| NAD-dependent 4-hydroxybutyrate | ZP_06983178 |
| dehydrogenase | |
| NAD-dependent 4-hydroxybutyrate | NP_904964 |
| dehydrogenase | |
| NAD-dependent 4-hydroxybutyrate | ZP_04389726 |
| dehydrogenase | |
| alcohol dehydrogenase, iron-dependent | ZP_07821131 |
| NAD-dependent 4-hydroxybutyrate | ZP_05427218 |
| dehydrogenase | |
| hypothetical protein CLOL250_02815 | ZP_02076027 |

TABLE 1O
Suitable homologues for the SsaR_{At2} protein (succinic
semialdehyde reductase, from Aspergillus terreus,
EC No. 1.1.1.61, which acts on succinate semialdehyde to
produce 4-hydroxybutyrate; protein acc. no. XP_001210625)

| | Protein |
|---|---|
| Protein Name | Accession No. |
| aflatoxin B1-aldehyde reductase, putative | XP_001268918 |
| aflatoxin B1-aldehyde reductase, putative | XP_001264422 |
| hypothetical protein An08g06440 | XP_001392759 |
| Pc13g11860 | XP_002559603 |
| TPA: aflatoxin B1-aldehyde reductase | CBF89011 |
| GliO-like, putative | |
| aflatoxin B1 aldehyde reductase | EEH21318 |
| aflatoxin B1 aldehyde reductase member, | XP_003069315 |
| putative | |
| aldo/keto reductase | XP_002625767 |
| aflatoxin B1 aldehyde reductase member 2 | XP_002845070 |

TABLE 1P
Suitable homologues for the SsaR_{Mm} protein (succinic semialdehyde
reductase, from Mus musculus, EC No. 1.1.1.61, which acts on succinate
semialdehyde to produce 4-hydroxybutyrate; protein acc. no. AKR7A5)

| Protein Name | Protein Accession No. |
|---|---|
| aflatoxin B1 aldehyde reductase | XP_001092177 |
| member 2 | |
| AKR7A2 protein | AAI49541 |
| similar to aflatoxin B1 aldehyde | XP_001917301 |
| reductase member 3 | |
| aldo-keto reductase family 7, member | XP_002685838 |
| A3 | |

TABLE 1Q
Suitable homologues for the YqhD protein (succinic semialdehyde
reductase, from Escherichia coli K-12, EC No. 1.1.1.61,
which acts on succinate semialdehyde to produce 4-hydroxybutyrate;
protein acc. no. NP_417484)

| Protein Name | Protein Accession No. |
|---|---|
| alcohol dehydrogenase yqhD | ZP_02900879 |
| alcohol dehydrogenase, NAD(P)- | YP_002384050 |
| dependent | |
| putative alcohol dehydrogenase | YP_003367010 |
| alcohol dehydrogenase YqhD | ZP_02667917 |
| putative alcohol dehydrogenase | YP_218095 |
| hypothetical protein ESA_00271 | YP_001436408 |
| iron-containing alcohol dehydrogenase | YP_003437606 |
| hypothetical protein CKO_04406 | YP_001455898 |
| alcohol dehydrogenase | ZP_03373496 |

TABLE 1R
Suitable homologues for the OrfZ protein (CoA transferase, from
Clostridium kluyveri DSM 555, EC No. 2.8.3.n, which acts on
4-hydroxybutyrate to produce 4-hydroxybutyryl CoA;
protein acc. no. AAA92344)

| Protein Name | Protein Accession No. |
|---|---|
| 4-hydroxybutyrate coenzyme A | YP_001396397 |
| transferase | |
| acetyl-CoA hydrolase/transferase | ZP_05395303 |
| acetyl-CoA hydrolase/transferase | YP_001309226 |
| 4-hydroxybutyrate coenzyme A | NP_781174 |
| transferase | |
| 4-hydroxybutyrate coenzyme A | ZP_05618453 |
| transferase | |
| acetyl-CoA hydrolase/transferase | ZP_05634318 |
| 4-hydroxybutyrate coenzyme A | ZP_00144049 |
| transferase | |
| hypothetical protein ANASTE_01215 | ZP_02862002 |
| 4-hydroxybutyrate coenzyme A | ZP_07455129 |
| transferase | |

TABLE 1S
Suitable homologues for the PhaC1 protein (polyhydroxyalkanoate
synthase, from Ralstonia eutropha H16, EC No. 2.3.1.n, which acts on
(R)-3-hydroxybutyryl-CoA or 4-hydroxybutyryl-CoA + [(R)-3-
hydroxybutanoate-co-4-hydroxybutanoate]ₙ to produce
[(R)-3-hydroxybutanoate-co-4-hydroxybutanoate]₍ₙ₊₁₎ +
CoA and also acts on 4-hydroxybutyryl-CoA + [4-hydroxybutanoate]ₙ
to produce [4-hydroxybutanoate]₍ₙ₊₁₎ + CoA; Protein ace. no.
YP_725940 (Peoples and Sinskey, J. Biol. Chem. 264: 15298-15303
(1989))).

| Protein Name | Protein Accession No. |
|---|---|
| polyhydroxyalkanoic acid synthase | YP_002005374 |
| PHB synthase | BAB96552 |
| PhaC | AAF23364 |
| Polyhydroxyalkanoate synthase protein | AAC83658 |
| PhaC | |
| polyhydroxybutyrate synthase | AAL17611 |
| poly(R)-hydroxyalkanoic acid synthase, | YP_002890098 |
| class I | |
| poly-beta-hydroxybutyrate polymerase | YP_159697 |
| PHB synthase | CAC41638 |
| PHB synthase | YP_001100197 |

TABLE 1T
Suitable homologues for the PhaC3/C1 protein (Polyhydroxyalkanoate
synthase fusion protein from Pseudomonas putida and Ralstonia
eutropha JMP134, EC No. 2.3.1.n, which acts on (R)-3-hydroxybutyryl-
CoA or 4-hydroxybutyryl-CoA + [(R)-3-hydroxybutanoate-co-4-
hydroxybutanoate]ₙ to produce [(R)-3-hydroxybutanoate-co-4-
hydroxybutanoate]₍ₙ₊₁₎ + CoA and also acts on 4-hydroxybutyryl-
CoA + [4-hydroxybutanoate]ₙ to produce [4-hydroxybutanoate]₍ₙ₊₁₎ + CoA

| Protein Name | Protein Accession No. |
|---|---|
| Poly(R)-hydroxyalkanoic acid synthase, | YP_295561 |
| class I | |
| Poly(3-hydroxybutyrate) polymerase | YP_725940 |
| polyhydroxyalkanoic acid synthase | AAW65074 |
| polyhydroxyalkanoic acid synthase | YP_002005374 |
| Poly(R)-hydroxyalkanoic acid synthase, | YP_583508 |
| class I | |
| intracellular polyhydroxyalkanoate | ADM24646 |
| synthase | |
| Poly(3-hydroxyalkanoate) polymerase | ZP_00942942 |
| polyhydroxyalkanoic acid synthase | YP_003752369 |
| PhaC | AAF23364 |

TABLE 1U
Suitable homologues for the Buk1 protein (butyrate kinase I, from
Clostridium acetobutylicum ATCC824, EC No. 2.7.2.7, which acts
on 4-hydroxybutyrate to produce 4-hydroxybutyryl phosphate

| Protein Name | Protein Accession No. |
|---|---|
| butyrate kinase | YP_001788766 |
| butyrate kinase | YP_697036 |
| butyrate kinase | YP_003477715 |
| butyrate kinase | YP_079736 |
| acetate and butyrate kinase | ZP_01667571 |
| butyrate kinase | YP_013985 |
| butyrate kinase | ZP_04670620 |
| butyrate kinase | ZP_04670188 |
| butyrate kinase | ZP_07547119 |

TABLE 1V
Suitable homologues for the Buk2 protein (butyrate kinase II, from
Clostridium acetobutylicum ATCC824, EC No. 2.7.2.7, which acts
on 4-hydroxybutyrate to produce 4-hydroxybutyryl phosphate

| Protein Name | Protein Accession No. |
|---|---|
| butyrate kinase | YP_001311072 |
| hypothetical protein CLOSPO_00144 | ZP_02993103 |
| hypothetical protein COPEUT_01429 | ZP_02206646 |
| butyrate kinase | EFR5649 |
| butyrate kinase | ZP_0720132 |
| butyrate kinase | YP_0029418 |
| butyrate kinase | YP_002132418 |
| butyrate kinase | ZP_05389806 |
| phosphate butyryltransferase | ADQ27386 |

TABLE 1W
Suitable homologues for the Ptb protein (phosphotransbutyrylase, from
Clostridium acetobutylicum ATCC824, EC No. 2.3.1.19, which acts
on 4-hydroxybutyryl phosphate to produce 4-hydroxybutyryl CoA

| Protein Name | Protein Accession No. |
|---|---|
| phosphate butyryltransferase | YP_001884531 |
| hypothetical protein COPCOM_01477 | ZP_03799220 |
| phosphate butyryltransferase | YP_00331697 |
| phosphate butyryltransferase | YP_004204177 |
| phosphate acetyl/butyryltransferase | ZP_05265675 |
| putative phosphate | ZP_05283680 |
| acetyl/butyryltransferase | |
| bifunctional enoyl-CoA | YP_426556 |
| hydratase/phosphate acetyltransferase | |
| hypothetical protein CLOBOL_07039 | ZP_02089466 |
| phosphate butyryltransferase | YP_003564887 |

TABLE 1X
Suitable homologues for the SucC protein (succinate-CoA ligase (ADP-
forming), beta subunit, from Escherichia coli K-12, EC No. 6.2.1.5,
which acts on succinate and CoA to produce succinyl-CoA

| Protein Name | Protein Accession No. |
|---|---|
| succinyl-CoA synthetase, beta chain | YP_003942629 |
| succinyl-CoA synthetase subunit beta | YP_003005213 |
| succinyl-CoA synthetase subunit beta | YP_002150340 |
| succinyl-CoA ligase (ADP-forming) | ZP_06124567 |
| succinyl-CoA synthetase subunit beta | YP_001187988 |
| succinyl-CoA synthetase subunit beta | ZP_01075062 |
| succinyl-CoA ligase (ADP-forming) | ZP_05984280 |
| succinyl-CoA synthetase subunit beta | YP_003699804 |
| succinyl-CoA synthetase subunit beta | YP_003443470 |

TABLE 1Y
Suitable homologues for the SucD protein (succinate-CoA ligase (ADP-
forming), alpha subunit, from Escherichia coli K-12, EC No. 6.2.1.5,
which acts on succinate and CoA to produce succinyl-CoA

| Protein Name | Protein Accession No. |
|---|---|
| succinyl-CoA synthetase subunit alpha | YP_402344 |
| succinate-CoA ligase | ZP_07949625 |
| succinyl-CoA synthetase subunit alpha | NP_792024 |
| succinyl-CoA synthetase, alpha subunit | YP_001784751 |
| succinyl-CoA synthetase alpha chain | ZP_03822017 |
| succinyl-CoA ligase | ZP_07004580 |
| hypothetical protein | XP_002872045 |
| ARALYDRAFT_489184 | |
| succinyl-CoA synthetase subunit alpha | YP_896208 |
| succinyl-CoA synthetase (ADP- | YP_611746 |
| forming) alpha subunit | |

TABLE 1Z
Suitable homologues for the Cat1 protein (succinyl-CoA:coenzyme A
transferase, from Clostridium kluyveri DSM 555, EC No. 2.8.3.n, which
acts on succinate and acetyl-CoA to produce succinyl-CoA and acetate

| Protein Name | Protein Accession No. |
|---|---|
| succinyl-CoA synthetase subunit | YP_402344 |
| alpha | |
| succinate-CoA ligase | ZP_07949625 |
| succinyl-CoA synthetase subunit | NP_792024 |
| alpha | |
| succinyl-CoA synthetase, alpha | YP_001784751 |
| subunit | |
| succinyl-CoA synthetase alpha chain | ZP_03822017 |
| succinyl-CoA ligase | ZP_07004580 |
| hypothetical protein | XP_002872045 |
| ARALYDRAFT_489184 | |
| succinyl-CoA synthetase subunit | YP_896208 |
| alpha | |
| succinyl-CoA synthetase (ADP- | YP_611746 |
| forming) alpha subunit | |

### Suitable Extrachromosomal Vectors and Plasmids

A “vector,” as used herein, is an extrachromosomal replicon, such as a plasmid, phage, or cosmid, into which another DNA segment may be inserted so as to bring about the replication of the inserted segment. Vectors vary in copy number and depending on the origin of their replication they contain, their size, and the size of insert. Vectors with different origin of replications can be propagated in the same microbial cell unless they are closely related such as pMB1 and ColE1. Suitable vectors to express recombinant proteins can constitute pUC vectors with a pMB1 origin of replication having 500-700 copies per cell, pBluescript vectors with a ColE1 origin of replication having 300-500 copies per cell, pBR322 and derivatives with a pMB1 origin of replication having 15-20 copies per cell, pACYC and derivatives with a p15A origin of replication having 10-12 copies per cell, and pSC101 and derivatives with a pSC101 origin of replication having about 5 copies per cell as described in the QIAGEN® Plasmid Purification Handbook (found on the world wide web at: //kirshner.med.harvard.edu/files/protocols/QIAGEN_QIAGENPlasmidPurification_EN.pdf.

### Suitable Strategies and Expression Control Sequences for Recombinant Gene Expression

Strategies for achieving expression of recombinant genes in E. coli have been extensively described in the literature (Gross, Chimica Oggi 7(3):21-29 (1989); Olins and Lee, Cur. Op. Biotech. 4:520-525 (1993); Makrides, Microbiol. Rev. 60(3):512-538 (1996); Hannig and Makrides, Trends in Biotech. 16:54-60 (1998)). Expression control sequences can include constitutive and inducible promoters, transcription enhancers, transcription terminators, and the like which are well known in the art. Suitable promoters include, but are not limited to, Plac, Ptac, Ptrc, PR, PL, Ptrp, PphoA, Para, PuspA, PrspU, Psyn (Rosenberg and Court, Ann. Rev. Genet. 13:319-353 (1979); Hawley and McClure, Nucl. Acids Res. 11 (8):2237-2255 (1983); Harley and Raynolds, Nucl. Acids Res. 15:2343-2361 (1987); also ecocyc.org and partsregistry.org.

### Construction of Recombinant Hosts

Recombinant hosts containing the necessary genes that will encode the enzymatic pathway for the conversion of a carbon substrate to P4HB may be constructed using techniques well known in the art.

Methods of obtaining desired genes from a source organism (host) are common and well known in the art of molecular biology. Such methods can be found described in, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999). For example, if the sequence of the gene is known, the DNA may be amplified from genomic DNA using polymerase chain reaction (Mullis, U.S. Pat. No. 4,683,202) with primers specific to the gene of interest to obtain amounts of DNA suitable for ligation into appropriate vectors. Alternatively, the gene of interest may be chemically synthesized de novo in order to take into consideration the codon bias of the host organism to enhance heterologous protein expression. Expression control sequences such as promoters and transcription terminators can be attached to a gene of interest via polymerase chain reaction using engineered primers containing such sequences. Another way is to introduce the isolated gene into a vector already containing the necessary control sequences in the proper order by restriction endonuclease digestion and ligation. One example of this latter approach is the BioBrick™ technology (see the World Wide Web at biobricks.org) where multiple pieces of DNA can be sequentially assembled together in a standardized way by using the same two restriction sites.

In addition to using vectors, genes that are necessary for the enzymatic conversion of a carbon substrate to P4HB can be introduced into a host organism by integration into the chromosome using either a targeted or random approach. For targeted integration into a specific site on the chromosome, the method generally known as Red/ET recombineering is used as originally described by Datsenko and Wanner (Proc. Natl. Acad. Sci. USA, 2000, 97, 6640-6645). Random integration into the chromosome involved using a mini-Tn5 transposon-mediated approach as described by Huisman et al. (U.S. Pat. Nos. 6,316,262 and 6,593,116).

### Culturing of Host to Produce P4HB Biomass

In general, the recombinant host is cultured in a medium with a carbon source and other essential nutrients to produce the P4HB biomass by fermentation techniques either in batches or continuously using methods known in the art. Additional additives can also be included, for example, antifoaming agents and the like for achieving desired growth conditions. Fermentation is particularly useful for large scale production. An exemplary method uses bioreactors for culturing and processing the fermentation broth to the desired product. Other techniques such as separation techniques can be combined with fermentation for large scale and/or continuous production.

As used herein, the term “feedstock” refers to a substance used as a carbon raw material in an industrial process. When used in reference to a culture of organisms such as microbial or algae organisms such as a fermentation process with cells, the term refers to the raw material used to supply a carbon or other energy source for the cells. Carbon sources useful for the production of GBL include simple, inexpensive sources, for example, glucose, sucrose, lactose, fructose, xylose, maltose, arabinose and the like alone or in combination. In other embodiments, the feedstock is molasses or starch, fatty acids, vegetable oils or a lignocelluloses material and the like. It is also possible to use organisms to produce the P4HB biomass that grow on synthesis gas (CO2, CO and hydrogen) produced from renewable biomass resources.

Introduction of P4HB pathway genes allows for flexibility in utilizing readily available and inexpensive feedstocks. A “renewable” feedstock refers to a renewable energy source such as material derived from living organisms or their metabolic byproducts including material derived from biomass, often consisting of underutilized components like chaff or stover. Agricultural products specifically grown for use as renewable feedstocks include, for example, corn, soybeans, switchgrass and trees such as poplar, wheat, flaxseed and rapeseed, sugar cane and palm oil. As renewable sources of energy and raw materials, agricultural feedstocks based on crops are the ultimate replacement of declining oil reserves. Plants use solar energy and carbon dioxide fixation to make thousands of complex and functional biochemicals beyond the current capability of modern synthetic chemistry. These include fine and bulk chemicals, pharmaceuticals, nutraceuticals, flavanoids, vitamins, perfumes, polymers, resins, oils, food additives, bio-colorants, adhesives, solvents, and lubricants.

Combining P4HB Biomass with a First Catalyst

In general, during or following production (e.g., culturing) of the P4HB biomass, the biomass is combined with a first catalyst under suitable conditions to help convert the P4HB polymer to high purity gamma-butyrolactone vapor. The catalyst (in solid or solution form) and biomass are combined for example by mixing, flocculation, centrifuging or spray drying, or other suitable method known in the art for promoting the interaction of the biomass and catalyst driving an efficient and specific conversion of P4HB to gamma-butyrolactone. In some embodiments, the biomass is initially dried, for example at a temperature between about 100° C. and about 150° C. and for an amount of time to reduce the water content of the biomass. The dried biomass is then re-suspended in water prior to combining with the catalyst. Suitable temperatures and duration for drying are determined for product purity and yield and can in some embodiments include low temperatures for removing water (such as between 25° C. and 150° C.) for an extended period of time or in other embodiments can include drying at a high temperature (e.g., above 450° C.) for a short duration of time. Under “suitable conditions” refers to conditions that promote the catalytic reaction. For example, under conditions that maximize the generation of the product gamma-butyrolactone such as in the presence of co-agents or other material that contributes to the reaction efficiency. Other suitable conditions include in the absence of impurities, such as metals or other materials that would hinder the reaction from progression.

As used herein, “catalyst” refers to a substance that initiates or accelerates a chemical reaction without itself being affected or consumed in the reaction. Examples of useful catalysts for degradation of PHA's include metal-type catalysts. In certain embodiments, the catalyst lowers the temperature for initiation of thermal decomposition and increases the rate of thermal decomposition at certain pyrolysis temperatures (e.g., about 200° C. to about 325° C.).

In some embodiments, the catalyst is a chloride, oxide, hydroxide, nitrate, phosphate, sulphonate, carbonate or stearate compound containing a metal ion. Examples of suitable metal ions include aluminum, antimony, barium, bismuth, cadmium, calcium, cerium, chromium, cobalt, copper, gallium, iron, lanthanum, lead, lithium, magnesium, molybdenum, nickel, palladium, potassium, silver, sodium, strontium, tin, tungsten, vanadium or zinc and the like. In some embodiments, the catalyst is an organic catalyst that is an amine, azide, enol, glycol, quaternary ammonium salt, phenoxide, cyanate, thiocyanate, dialkyl amide and alkyl thiolate. In some embodiments, the catalyst is calcium hydroxide. In other embodiments, the catalyst is sodium carbonate. Mixtures of two or more catalysts are also included.

In certain embodiments, the amount of metal catalyst is about 0.1% to about 15% or about 1% to about 25%, or 4% to about 50%, or about 4% to about 50% based on the weight of metal ion relative to the dry solid weight of the biomass. In some embodiments, the amount of catalyst is between about 7.5% and about 12%. In other embodiments, the amount of catalyst is about 0.5% dry cell weight, about 1%, about 2%, about 3%, about 4%, about 5, about 6%, about 7%, about 8%, about 9%, or about 10%, or about 11%, or about 12%, or about 13%, or about 14%, or about 15%, or about 20%, or about 30%, or about 40% or about 50% or amounts in between these.

As used herein, the term “sufficient amount” when used in reference to a chemical reagent in a reaction is intended to mean a quantity of the reference reagent that can meet the demands of the chemical reaction and the desired purity of the product.

### Thermal Degradation of the P4HB Biomass

“Heating,” “pyrolysis”, “thermolysis” and “torrefying” as used herein refer to thermal degradation (e.g., decomposition) of the P4HB biomass for conversion to GBL. In general, the thermal degradation of the P4HB biomass occurs at an elevated temperature in the presence of a catalyst. For example, in certain embodiments, the heating temperature for the processes described herein is between about 200° C. to about 400° C. In some embodiments, the heating temperature is about 200° C. to about 350° C. In other embodiments, the heating temperature is about 300° C. “Pyrolysis” typically refers to a thermochemical decomposition of the biomass at elevated temperatures over a period of time. The duration can range from a few seconds to hours. In certain conditions, pyrolysis occurs in the absence of oxygen or in the presence of a limited amount of oxygen to avoid oxygenation. The processes for P4HB biomass pyrolysis can include direct heat transfer or indirect heat transfer. “Flash pyrolysis” refers to quickly heating the biomass at a high temperature for fast decomposition of the P4HB biomass, for example, depolymerization of a P4HB in the biomass. Another example of flash pyrolysis is RTP™ rapid thermal pyrolysis. RTP™ technology and equipment from Envergent Technologies, Des Plaines, Ill. converts feedstocks into bio-oil. “Torrefying” refers to the process of torrefaction, which is an art-recognized term that refers to the drying of biomass at elevated temperature with loss of water and organic volatiles to produce a torrefied biomass with enhanced solid fuel properties. The torrefied biomass typically has higher heating value, greater bulk density, improved grindability for pulverized fuel boilers, increased mold resistance and reduced moisture sensitivity compared to biomass dried to remove free water only (e.g. conventional oven drying at 105° C.). The torrefaction process typically involves heating a biomass in a temperature range from 200-350° C., over a relatively long duration (e.g., 10-30 minutes), typically in the absence of oxygen. The process results for example, in a torrefied biomass having a water content that is less than 7 wt % of the biomass (e.g., less than 5%). The torrefied biomass may then be processed further. In some embodiments, the heating is done in a vacuum, at atmospheric pressure or under controlled pressure. In certain embodiments, the heating is accomplished without the use or with a reduced use of petroleum generated energy.

In certain embodiments, the P4HB biomass is dried prior to heating. Alternatively, in other embodiments, drying is done during the thermal degradation (e.g., heating, pyrolysis or torrefaction) of the P4HB biomass. Drying reduces the water content of the biomass. In certain embodiments, the biomass is dried at a temperature of between about 100° C. to about 350° C., for example, between about 200° C. and about 275° C. In some embodiments, the dried 4PHB biomass has a water content of 5 wt %, or less.

In certain embodiments, the heating of the P4HB biomass with the first catalyst is carried out for a sufficient time to efficiently and specifically convert the P4HB biomass to GBL vapor. In certain embodiments, the time period for heating is from about 30 seconds to about 1 minute, from about 30 seconds to about 1.5 minutes, from about 1 minute to about 10 minutes, from about 1 minute to about 5 minutes or a time between, for example, about 1 minute, about 2 minutes, about 1.5 minutes, about 2.5 minutes, about 3.5 minutes.

In other embodiments, the time period is from about 1 minute to about 2 minutes. In still other embodiments, the heating time duration is for a time between about 5 minutes and about 30 minutes, between about 30 minutes and about 2 hours, or between about 2 hours and about 10 hours or for greater that 10 hours (e.g., 24 hours).

In certain embodiments, the heating temperature is at a temperature of about 200° C. to about 350° C. including a temperature between, for example, about 205° C., about 210° C., about 215° C., about 220° C., about 225° C., about 230° C., about 235° C., about 240° C., about 245° C., about 250° C., about 255° C. about 260° C., about 270° C., about 275° C., about 280° C., about 290° C., about 300° C., about 310° C., about 320° C., about 330° C., about 340° C., or 345° C. In certain embodiments, the temperature is about 250° C. In certain embodiments, the temperature is about 275° C. In other embodiments, the temperature is about 300° C.

In certain embodiments, the process also includes flash pyrolyzing the residual biomass for example at a temperature of about 500° C. or greater for a time period sufficient to decompose at least a portion of the residual biomass into pyrolysis liquids. In certain embodiments, the flash pyrolyzing is conducted at a temperature of about 500° C. to about 750° C. In some embodiments, a residence time of the residual biomass in the flash pyrolyzing is from 1 second to 15 seconds, or from 1 second to 5 seconds or for a sufficient time to pyrolyze the biomass to generate the desired pyrolysis precuts, for example, pyrolysis liquids. In some embodiments, the flash pyrolysis can take place instead of torrefaction. In other embodiments, the flash pyrolysis can take place after the torrrefication process is complete.

As used herein, “pyrolysis liquids” are defined as a low viscosity fluid with up to 15-20% water, typically containing sugars, aldehydes, furans, ketones, alcohols, carboxylic acids and lignins. Also known as bio-oil, this material is produced by pyrolysis, typically fast pyrolysis of biomass at a temperature that is sufficient to decompose at least a portion of the biomass into recoverable gases and liquids that may solidify on standing. In some embodiments, the temperature that is sufficient to decompose the biomass is a temperature between 400° C. to 800° C.

In certain embodiments, the GBL vapor produced from heating P4HB biomass is first partially condensed to remove higher boiling compounds like bio-oil as previously described. As a non-limiting example, the partial condensation of the gamma-butyrolactone vapor may be described as follows: the incoming gas/vapor stream from the pyrolysis/torrefaction chamber enters an interchanger, where the gas/vapor stream may be pre-cooled. The gas/vapor stream then passes through a heat exchanger where the temperature of the gas/vapor stream is lowered to that required to condense the high boiling vapors from the GBL gas by indirect contact with a cooling medium. In certain embodiments, the cooling medium can be water and the heat from the gas/vapor stream is captured in the form of steam that is used in the process improving the overall energy efficiency of the process. In other embodiments the cooling medium can simply be cooling water or other suitable refrigerant. The GBL gas and condensed vapors flow from the heat exchanger into a separator, where the condensed vapors are collected in the bottom. The GBL gas, free of the bio-oil vapors, flows from the separator and exits the unit.

Vapor Phase Hyrdogenation with a Second Catalyst

In a further embodiment, the GBL gas is processed to tetrahydrofuran liquid by vapor phase hydrogenation using a second catalyst. The GBL gas is first passed over a guard bed column containing an absorbent designed to remove organic nitrogen impurities (generated from the biomass) which would otherwise poison the hydrogenation catalyst. The absorbent comprises activated carbon, clay, alumina, zeolite, silica gel or any mixture of these as outlined in U.S. Pat. No. 7,867,381 (Koseoglu, assigned to Saudia. Arabian Oil Company, 2011). The purified GBL gas is then compressed to a pressure of about 8 bars, about 7 bars, about 6 bars, about 5 bars, about 4 bars about 3 bars, or about 2 bars, mixed with 99% pure hydrogen gas at a composition ratio of H₂/GBL of about 99%/1%, about 98%/2%, about 97%/3%, about 96%/4% or about 95%/5%, at a temperature of 190° C. and fed to a vapor phase catalytic bed reactor. The reactor is a tube-type which contains a hydrogenation catalyst in a fixed bed arrangement. The catalyst can be comprised of a mixture of copper oxide, zinc oxide, aluminum oxide, chrome oxide, cobalt oxide and/or nickel oxide compounds stabilized on an inert substrate as described in U.S. Pat. No. 4,006,104 (Michalczyk et al. assigned to Deutsche Texaco Aktiengesellschaft, 1977), U.S. Pat. No. 5,149,836 (De Thomas et al. assigned to ISP, 1992) and U.S. Pat. No. 6,831,182 (Borchert et al. assigned to BASF Aktiengesellschaft, 2004). Preferred catalyst mixtures are 20-40% CuO/20-40% ZnO/5-20% Al₂O₃ for carrying out the vapor phase hydrogenation of GBL. Prior to introducing the H₂/GBL mixture, the second catalyst is initially activated by heating it first in the presence of nitrogen gas and then pure hydrogen (99%) using a well defined temperature profile. After catalyst activation, the hydrogen/GBL mixture is then introduced into the reactor, passed over the catalyst bed at about 180° C., about 190° C., about 200° C. or about 210° C. to promote the hydrogenation reaction. After hydrogenation, the THF is recovered as a liquid by condensation as described previously and then optionally distilled to separate the pure compounds. The advantage of in situ conversion of GBL gas is that it greatly improves the energetics of the process as it avoids the energy intensive operations of first condensing GBL and subsequently vaporizing the same GBL before feeding to the catalytic reactor. By integrating the thermolysis and downstream catalytic conversion it is now possible to produce renewable THF directly from biomass containing P4HB at the same time eliminating costly and energy intensive unit operations.

In certain embodiments, recovery of the first catalyst is further included in the processes of the invention. For example, when a calcium catalyst is used calcination is a useful recovery technique. Calcination is a thermal treatment process that is carried out on minerals, metals or ores to change the materials through decarboxylation, dehydration, devolatilization of organic matter, phase transformation or oxidation. The process is normally carried out in reactors such as hearth furnaces, shaft furnaces, rotary kilns or more recently fluidized beds reactors. The calcination temperature is chosen to be below the melting point of the substrate but above its decomposition or phase transition temperature. Often this is taken as the temperature at which the Gibbs free energy of reaction is equal to zero. For the decomposition of CaCO₃ to CaO, the calcination temperature at ΔG=0 is calculated to be ˜850° C. Typically for most minerals, the calcination temperature is in the range of 800-1000° C. but calcinations can also refer to heating carried out in the 200-800° C. range.

To recover the calcium catalyst from the biomass after recovery of the GBL, one would transfer the spent biomass residue directly from pyrolysis or torrefaction into a calcining reactor and continue heating the biomass residue in air to 825-850° C. for a period of time to remove all traces of the organic biomass. Once the organic biomass is removed, the catalyst could be used as is or purified further by separating the metal oxides present (from the fermentation media and catalyst) based on density using equipment known to those in the art.

In certain embodiments, the process is selective for producing gamma-butyrolactone product with a relatively small amount of undesired side products (e.g., dimerized product of GBL (3-(dihydro-2(3H)-furanylidene)dihydro-2(3H)-furanone), other oligomers of GBL or other side products). For example, in some embodiments the use of a first catalyst in a sufficient amount will reduce the production of undesired side products and increase the yield of gamma-butyrolactone vapor by at least about 2 fold. In some embodiments, the production of undesired side products will be reduced to at least about 50%, at least about 40%, at least about 30%, at least about 20% at least about 10%, or about at least 5%. In certain embodiment, the undesired side products will be less than about 5% of the recovered gamma-butyrolactone, less than about 4% of the recovered gamma-butyrolactone, less than about 3% of the recovered gamma-butyrolactone, less than about 2% of the recovered gamma-butyrolactone, or less than about 1% of the recovered gamma-butyrolactone.

The processes described herein can provide a yield of THF expressed as a percent yield, for example, when prepared from glucose as a carbon source, the mass yield is up to 80% based on grams THF recovered per gram P4HB contained in the biomass feed to the process (equivalent to a molar yield of 0.95 mol THF recovered per mol P4HB on a monomer basis contained in the biomass feed). In other embodiments, the mass yield is in a range between about 40% and about 80%, for example between about 50% and about 70%, or between about 60% and 70%. In other embodiment, the yield is about 75%, about 70%, about 65%, about 60%, about 55%, about 50%, about 45% or about 40%.

As used herein, “gamma-butyrolactone” or GBL refers to the compound with the following chemical structure:

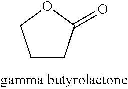

As used herein, “tetrahydrofuran” or THF refers to the compound with the following chemical structure:

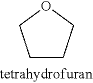

The term “tetrahydrofuran product” refers to a product that contains at least about 70 up to 100 weight percent tetrahydrofuran. For example, in a certain embodiment, the tetrahydrofuran product may contain 95% by weight tetrahydrofuran and 5% by weight side products. In some embodiments, the amount of tetrahydrofuran in the tetrahydrofuran product is about 71% by weight, about 72% by weight, about 73% by weight, about, 74% by weight, about 75% by weight, about 76% by weight, about 77% by weight, about 78% by weight, about 79% by weight, about 80% by weight, 81% by weight, about 82% by weight, about 83% by weight, about, 84% by weight, about 85% by weight, about 86% by weight, about 87% by weight, about 88% by weight, about 89% by weight, about 90% by weight, 91% by weight, about 92% by weight, about 93% by weight, about, 94% by weight, about 95% by weight, about 96% by weight, about 97% by weight, about 98% by weight, about 99% by weight or about 100% by weight. In particular embodiments, the weight percent of tetrahydrofuran product produced by the processes described herein is 85% or greater than 85%.

In other embodiments, the tetrahydrofuran product can be further purified if needed by additional methods known in the art, for example, by distillation, by reactive distillation (e.g., the gamma-butyrolactone product is acidified first to oxidize certain components (e.g., for ease of separation) and then distilled) by treatment with activated carbon for removal of color and/or odor bodies, by ion exchange treatment, by liquid-liquid extraction—with GBL immiscible solvent (e.g., nonpolar solvents, like cyclopentane or hexane) to remove fatty acids etc, for purification after GBL recovery, by vacuum distillation, by extraction distillation or using similar methods that would result in further purifying the tetrahydrofuran product to increase the yield of tetrahydrofuran. Combinations of these treatments can also be utilized.

As used herein, the term “residual biomass” refers to the biomass after PHA conversion to the small molecule intermediates. The residual biomass may then be converted via torrefaction to a useable, fuel, thereby reducing the waste from PHA production and gaining additional valuable commodity chemicals from typical torrefaction processes. The torrefaction is conducted at a temperature that is sufficient to densify the residual biomass. In certain embodiments, processes described herein are integrated with a torrefaction process where the residual biomass continues to be thermally treated once the volatile chemical intermediates have been released to provide a fuel material. Fuel materials produced by this process are used for direct combustion or further treated to produce pyrolysis liquids or syngas. Overall, the process has the added advantage that the residual biomass is converted to a higher value fuel which can then be used for the production of electricity and steam to provide energy for the process thereby eliminating the need for waste treatment.

A “carbon footprint” is a measure of the impact the processes have on the environment, and in particular climate change. It relates to the amount of greenhouse gases produced.

In certain embodiments, it may be desirable to label the constituents of the biomass. For example, it may be useful to deliberately label with an isotope of carbon (e.g., 13C) to facilitate structure determination or for other means. This is achieved by growing microorganisms genetically engineered to express the constituents, e.g., polymers, but instead of the usual media, the bacteria are grown on a growth medium with 13C-containing carbon source, such as glucose, pyruvic acid, etc. In this way polymers can be produced that are labeled with 13C uniformly, partially, or at specific sites. Additionally, labeling allows the exact percentage in bioplastics that came from renewable sources (e.g., plant derivatives) can be known via ASTM D6866—an industrial application of radiocarbon dating. ASTM D6866 measures the Carbon 14 content of biobased materials; and since fossil-based materials no longer have Carbon 14, ASTM D6866 can effectively dispel inaccurate claims of biobased content

### EXAMPLES

The present technology is further illustrated by the following examples, which should not be construed as limiting in any way.

### Experimental Methods

### Measurement of Thermal Degradation Behavior by Thermogravimetric Analysis (TGA)

The isothermal weight loss versus time for biomass samples was measured using a TA Instruments Q500 Thermogravimetric Analyzer (TGA). TGA is a technique commonly used to measure the thermal degradation behavior of materials such as PHA's. The instrument consists of a sensitive balance from which a sample is suspended. A furnace is then brought up around the sample and programmed to heat at a specified rate (ramp conditions) or to a certain temperature and hold (isothermal conditions). A purge gas is swept across the sample during heating which is typically nitrogen or air. As the sample is heated, it begins to lose weight which is recorded by the balance. At the end of the analysis, the results can then be plotted as percent sample weight loss versus temperature or time. When plotted as weight loss versus time, the rate of degradation can then be determined from the slope of this curve. For the following examples, 5-10 mg of dry biomass was weighed into a platinum pan and then loaded onto the TGA balance. The purge gas used was nitrogen at a flow rate of 60 ml/min. For isothermal test conditions, the biomass sample was preheated from room temperature to the programmed isothermal temperature at a heating rate of 150-200° C./min and held at the isothermal temperature for 10-30 min. The data was then plotted as % sample weight loss vs. time and the thermal degradation rate calculated from the initial slope of the curve.

### Measurement of Thermal Degradation Products by Pyrolysis-Gas Chromatography-Mass Spectroscopy (Py-GC-MS).

In order to identify and semi-quantitate the monomer compounds generated from dry biomass while being heated at various temperatures, an Agilent 7890A/5975 GC-MS equipped with a Frontier Lab PY-20201D pyrolyzer was used. For this technique, a sample is weighed into a steel cup and loaded into the pyrolyzer autosampler. When the pyrolyzer and GC-MS are started, the steel cup is automatically placed into the pyrolyzer which has been set to a specific temperature. The sample is held in the pyrolyzer for a short period of time while volatiles are released by the sample. The volatiles are then swept using helium gas into the GC column where they condense onto the column which is at room temperature. Once the pyrolysis is over, the GC column is heated at a certain rate in order to elute the volatiles released from the sample. The volatile compounds are then swept using helium gas into an electro ionization/mass spectral detector (mass range 10-700 daltons) for identification and quantitation.

For the following examples, 200-400 μg of dry biomass was weighed into a steel pyrolyzer cup using a microbalance. The cup was then loaded into the pyrolyzer autosampler. The pyrolyzer was programmed to heat to temperatures ranging from 225-350° C. for a duration of 0.2-1 minutes. The GC column used in the examples was either a Frontier Lab Ultra Alloy capillary column or an HP-5MS column (length 30m, ID 0.25 μm, film thickness 0.25 μm). The GC was then programmed to heat from room temperature to 70° C. over 5 minutes, then to 240° C. at 10° C./min for 4 min. and finally to 270° C. at 20° C./min for 1.5 min. Total GC run time was 25 minutes. Peaks showing in the chromatogram were identified by the best probability match to spectra from a NIST mass spectral library. GBL ‘purity’ was measured by taking the area counts for GBL peak and dividing it by the area counts for GBL dimer peak.

These examples describe a number of biotechnology tools and methods for the construction of strains that generate a product of interest. Suitable host strains, the potential source and a list of recombinant genes used in these examples, suitable extrachromosomal vectors, suitable strategies and regulatory elements to control recombinant gene expression, and a selection of construction techniques to overexpress genes in or inactivate genes from host organisms are described. These biotechnology tools and methods are well known to those skilled in the art.

### Example 1

### 4HB Polymer Production before Modification

This example shows the 411B polymer production capability of microbial strains have not been optimized to incorporate high mole % 4HB from renewable carbon resources. The strains used in this example are listed in Table 2. Strains 1 and 2 were described by Dennis and Valentin (U.S. Pat. No. 6,117,658).

TABLE 2
Strains used in Example 1

| | Relevant host genome | |
|---|---|---|
| Strains | modifications | Genes overexpressed |
| 1 | | P_{tac}-phaCAB |
| | | P_{lac}-orfZ-′cat1-sucD-4hbD |
| 2 | yneI-negative | P_{tac}-phaCAB |
| | | P_{lac}-orfZ-′cat1-sucD-4hbD |
| 3 | ΔyneI ΔgabD | P_{X}-phaC, P₁₂-phaAB |
| | | P_{lac}-orfZ-′cat1-sucD-4hbD |

Strain 3 contained deletions of both the yneI and gabD chromosomal genes (FIG. 1 and Table 1A, Reaction Number 12) which encode the CoA-independent, NAD-dependent succinate semialdehyde (SSA) dehydrogenase and the CoA-independent, NADP-dependent SSA dehydrogenase, respectively. To accomplish this, a derivative strain of LS5218 (Jenkins and Nunn J. Bacteriol. 169:42-52 (1987)) was used that expressed phaA, phaB and phaC as described previously by Huisman et al. (U.S. Pat. No. 6,316,262). Single null gabD and yneI mutants were constructed as described by Farmer et al. (WO Patent No. 2010/068953) and used the Red/ET recombineering method described by Datsenko and Wanner (Proc. Natl. Acad. Sci. USA. 97:6640-6645 (2000)), a method well known to those skilled in the art. This resulted in strain 3 that had the entire coding sequences of both the yneI and gabD genes removed from the genome. Note that strains 1, 2, and 3 contain the same gene cassette Plac-orfZ-'cat1-sucD-4-hbD as described by Dennis and Valentin, where sucD is not codon-optimized for expression in E. coli.

To examine production of P3HB-co-4HB (poly-3-hydroxybutyrate-co-4-hydroxybutyrate), strain 3 was cultured overnight in a sterile tube containing 3 mL of LB and appropriate antibiotics. From this, 50 μL was added in triplicate to Duetz deep-well plate wells containing 450 □L of LB and antibiotics. This was grown for 6 hours at 30° C. with shaking. Then, 25 □L of each LB culture replicate was added to 3 additional wells containing 475 μL of LB medium supplemented with 10 g/L glucose, 100 □M IPTG, 100 μg/mL ampicillin, and 25 μg/mL chloramphenicol, and incubated at 30° C. with shaking for 72 hours. Thereafter, production well sets were combined (1.5 mL total) and analyzed for polymer content. At the end of the experiment, cultures were spun down at 4150 rpm, washed once with distilled water, frozen at −80° C. for at least 30 minutes, and lyophilized overnight. The next day, a measured amount of lyophilized cell pellet was added to a glass tube, followed by 3 mL of butanolysis reagent that consists of an equal volume mixture of 99.9% n-butanol and 4.0 N HCl in dioxane with 2 mg/mL diphenylmethane as internal standard. After capping the tubes, they were vortexed briefly and placed on a heat block set to 93° C. for six hours with periodic vortexing. Afterwards, the tube was cooled down to room temperature before adding 3 mL distilled water. The tube was vortexed for approximately 10 s before spinning down at 620 rpm (Sorvall Legend RT benchtop centrifuge) for 2 min 1 mL of the organic phase was pipetted into a GC vial, which was then analyzed by gas chromatography-flame ionization detection (GC-FID) (Hewlett-Packard 5890 Series II). The quantity of PHA in the cell pellet was determined by comparing against a standard curve for 4HB (for P4HB analysis) or by comparing against standard curves for both 3HB and 4HB (for PHB-co-4HB analysis). The 4HB standard curve was generated by adding different amounts of a 10% solution of □-butyrolactone (GBL) in butanol to separate butanolysis reactions. The 3HB standard curve was generated by adding different amounts of 99% ethyl 3-hydroxybutyrate to separate butanolysis reactions.

The results in Table 3 show that strain 3 incorporated similarly low mole % 4HB into the copolymer as was described in U.S. Pat. No. 6,117,658.

TABLE 3
P3HB-co-4HB polymer production from microbial strains

| Strains | Mole % 3HB | Mole % 4HB |
|---|---|---|
| 1 | 98.5 | 1.5 |
| 2 | 95.0 | 5.0 |
| 3 | 97.6 ± 0.9 | 2.4 ± 0.9 |

### Example 2

### P4HB Production Via an α-Ketoglutarate Decarboxylase or a Succinyl-CoA Dehydrogenase

Several metabolic pathways were proposed to generate succinic semialdehyde (SSA) from the tricarboxylic acid (TCA) cycle (reviewed by Steinbuchel and Lütke-Eversloh, Biochem. Engineering J. 16:81-96 (2003) and Efe et al., Biotechnology and Bioengineering 99:1392-1406 (2008). One pathway converts succinyl-CoA to SSA via a succinyl-CoA dehydrogenase, which is encoded by sucD (Söhling and Gottschalk, J. Bacterial. 178:871-880 (1996); FIG. 1, Reaction number 7). A second pathway converts alpha-ketoglutarate to SSA via an alpha-ketoglutarate decarboxylase that is encoded by kgdM (Tian et al. Proc. Natl. Acad. Sci. U.S.A. 102:10670-10675 (2005); FIG. 1, Reaction number 8). A third pathway converts alpha-ketoglutarate to SSA via L-glutamate and 4-aminobutyrate using a glutamate dehydrogenase (EC 1.4.1.4), a glutamate decarboxylase (EC 4.1.1.15), and a 4-aminobutyrate transaminase (EC 2.6.1.19), or a 4-aminobutyrate aminotransferase (EC 2.6.1.19). Van Dien et al. (WO Patent No. 2010/141920) showed that both the sucD and the kgdM pathways worked independently of each other and were additive when combined to produce 4HB. Note that kgdM is called sucA in van Dien et al.

In this example, the two metabolic pathways via sucD or kdgM were compared to see which one could produce the highest P4HB titers. The following three strains were thus constructed using the well known biotechnology tools and methods described above, all of which contained chromosomal deletions of yneI and gabD and overexpressed a PHA synthase, and a CoA transferase, and either an alpha-ketoglutarate decarboxylase with an SSA reductase (strain 5), or a succinyl-CoA dehydrogenase with an SSA reductase (strain 6). Strain 4 served as a negative control and just contained the empty vector instead of Ptrc-kgdM-ssaRAt* or Ptrc-sucD*-ssaRAt* (see Table 4).

TABLE 4
Microbial Strains used in Example 2

| | Relevant host | |
|---|---|---|
| | genome | |
| Strains | modifications | Genes overexpressed |
| 4 | ΔyneI ΔgabD | P_{rpsU}-orfZ; P_{syn1}-phaC1 |
| 5 | ΔyneI ΔgabD | P_{rpsU}-orfZ; P_{syn1}-phaC1; P_{trc}-kgdM-ssaR_{At}* |
| 6 | ΔyneI ΔgabD | P_{rpsU}-orfZ; P_{syn1}-phaC1; P_{trc}-sucD*-ssaR_{At}* |

The strains were grown in a 24 hour shake plate assay. The production medium consisted of 1× E2 minimal salts solution containing 10 g/L glucose, 5 g/L sodium 4-hydroxybutyrate, 2 mM MgSO4, 1× Trace Salts Solution, and 100 μM IPTG. 50× E2 stock solution consists of 1.275 M NaNH₄HPO₄.4H2O, 1.643 M K₂HPO₄, and 1.36 M KH₂PO₄. 1000× stock Trace Salts Solution is prepared by adding per 1 L of 1.5 NHCL: 50 g FeSO₄.7H₂O, 11 g ZnSO₄.7H2O, 2.5 g MnSO₄.4H₂O, 5 g CuSO₄.5H₂O, 0.5 g (NH₄)6Mo7O24.4H₂O, 0.1 g Na₂B₄O₇, and 10 g CaCl₂.2H₂O. At the end of the growth phase, the biomass and P4HB titers were determined as described in Example 1.

The results in Table 5 surprisingly show that only strain 6 expressing the sucD pathway produced significant amounts of P4HB. In contrast to the strains described by van Dien et al. (WO Patent No. 2010/141920) that produced 4HB via both the kgdM and sucD pathways in similar amounts, the alpha-ketoglutarate decarboxylase pathway used here produced only very low amounts of P4HB.

TABLE 5
Biomass and P4HB titer

| Strains | Biomass Titer (g/L) | P4HB Titer (% dcw) |
|---|---|---|
| 4 | 2.33 ± 0.02 | 0.0 ± 0.0 |
| 5 | 2.06 ± 0.03 | 0.1 ± 0.0 |
| 6 | 2.59 ± 0.01 | 6.9 ± 0.1 |

### Example 3

### Improvement in P(4HB) Production by Overexpressing Certain Succinic Semialdehyde Reductase Genes

### Effect of 4hbD on P4HB Production

The succinic semialdehyde (SSA) reductase gene 4hbD was used by Dennis and Valentin (U.S. Pat. No. 6,117,658) to produce P3HB-co-4HB copolymer. To see how effective overproduction of this SSA reductase was for P4HB homopolymer production, the 4hbD gene was overexpressed by the IPTG-inducible Ptrc promoter (strain 8). An empty vector containing strain served as a control (strain 7). The host strain used contained chromosomal deletions of genes yneI and gabD and also overexpressed the recombinant genes orfZ, sucD* and phaC3/C1* as shown in Table 6.

TABLE 6
Microbial Strains used in this section of Example 3

| | Relevant host | |
|---|---|---|
| | genome | |
| Strains | modifications | Genes overexpressed |
| 7 | ΔyneI ΔgabD | P_{rpsU}-orfZ, P_{uspA}-phaC3/C1*-sucD* |
| 8 | ΔyneI ΔgabD | P_{rpsU}-orfZ, P_{uspA}-phaC3/C1*-sucD*, |
| | | P_{trc}-4hbD |

The strains were grown in a 48 hour shake plate assay. The production medium consisted of 1× E2 minimal salts solution containing 20 g/L glucose, 1× Trace Salts Solution and 100 μM IPTG. Both E2 medium and trace elements are described in Example 2. At the end of the growth phase, the biomass and P4HB titers were determined as described in Example 1.

As shown in Table 7, strain 8 expressing 4hbD incorporated low amounts of 4HB into the polymer, similar to the strains described in U.S. Pat. No. 6,117,658 and verified in Example 1. However, very unexpectedly, the empty vector control strain 7, which did not express the 4hbd gene, produced significantly increased P4HB titers.

TABLE 7
Biomass and P4HB titer for microbial strains 7 and 8

| Strains | Biomass Titer (g/L) | P4HB Titer (% dcw) |
|---|---|---|
| 7 | 2.64 ± 0.04 | 17.09 ± 0.06 |
| 8 | 4.20 ± 0.09 | 3.17 ± 0.24 |

### Effect of Other SSA Reductase Genes on P4HB Production

Since the 4hbD-encoded SSA reductase unexpectedly did not produce higher amounts of P4HB than its parental strain, another known SSA reductase from Arabidopsis thaliana (Breitkreuz et al., J. Biol. Chem. 278:41552-41556 (2003)) was cloned in search of a catalytically more active enzyme. In addition, several genes whose protein sequences were found to be homologous to the A. thaliana enzyme were tested. These included putative SSA reductase genes from Mus musculus and Aspergillus terreus. Furthermore, to investigate if an unspecific aldehyde dehydrogenase from E. coli that did not show significant homology to the Arabidopsis enzyme could catalyze the SSA to 4HB reaction, gene yqhD was also cloned. YqhD was shown previously to have a catalytic activity to convert 3-hydroxypropionaldehyde to 1,3-propanediol (Emptage et al., U.S. Pat. No. 7,504,250). The resulting strains are listed in Table 8.

TABLE 8
Microbial strains used in Example 3

| | Relevant | |
|---|---|---|
| | host genome | |
| Strains | modifications | Genes overexpressed |
| 9 | ΔyneI ΔgabD | P_{rpsU}-orfZ, P_{uspA}-phaC3/C1*-sucD* |
| 10 | ΔyneI ΔgabD | P_{rpsU}-orfZ, P_{uspA}-phaC3/C1*-sucD*, P_{trc}- |
| | | ssaR_{At}* |
| 11 | ΔyneI ΔgabD | P_{rpsU}-orfZ, P_{uspA}-phaC3/C1*-sucD*, P_{trc}- |
| | | ssaR_{Mm}* |
| 12 | ΔyneI ΔgabD | P_{rpsU}-orfZ, P_{uspA}-phaC3/C1*-sucD*, P_{trc}- |
| | | ssaR_{At2}* |
| 13 | ΔyneI ΔgabD | P_{rpsU}-orfZ, P_{uspA}-phaC3/C1*-sucD*, P_{trc}- |
| | | yqhD |

Strains 9 to 13 were grown and the biomass and P4HB titers were determined as described above. Table 9 shows that unlike the 4hbD-encoded SSA reductase, overproduction of the SSA reductase from A. thaliana significantly increased P4HB production. This clearly illustrates how unpredictable the metabolic engineering outcome is albeit the known function of both the C. kluyveri and A. thaliana enzymes. The putative SSA reductase genes from M. musculus and A. terreus also improved P4HB production to various degrees. Unexpectedly, the unspecific E. coli aldehyde dehydrogenase YqhD increased P4HB production to a similar degree as was observed for the A. thaliana SSA reductase.

TABLE 9
Biomass and P4HB titer for microbial strains 9-13

| Strains | Biomass Titer (g/L) | P4HB Titer (% dcw) |
|---|---|---|
| 9 | 2.64 ± 0.04 | 17.09 ± 0.06 |
| 10 | 4.80 ± 0.12 | 28.46 ± 0.65 |
| 11 | 3.96 ± 0.79 | 23.31 ± 4.32 |
| 12 | 3.60 ± 0.29 | 19.74 ± 0.43 |
| 13 | 5.07 ± 0.07 | 27.99 ± 1.36 |

### Example 4

### Improved P4HB Production by Deletion of Pyruvate Kinases

Removal of pyruvate kinase I encoded by pykF and pyruvate kinase II encoded by pykA (FIG. 1, Reaction number 2) has been shown to reduce the production of acetate and favor the generation of CO₂ (Zhu et al. (2001) Biotechnol. Prog. 17:624-628). These results indicate that removal of pykF and pykA causes carbon flux to be diverted to the TCA cycle, and so these genetic modifications have been described as being useful for the microbial production of succinate and 1,4-butanediol (Park et al., WO Patent No. 2009/031766). To determine if deleting the pyruvate kinase genes pykF and pykA would lead to improved P4HB titers, the following two strains were constructed using the well known biotechnology tools and methods described above. Both of these strains contained chromosomal deletions of yneI and gabD and overexpressed a PHA synthase, a succinyl-CoA dehydrogenase, an SSA reductase and a CoA-transferase. Strain 14 retained its native unmodified copies of pykF and pykA on the chromosome, while strain 15 has both of these genes removed (Table 10).

TABLE 10
Microbial strains used in Example 4

| | Relevant | |
|---|---|---|
| | host genome | |
| Strains | modifications | Genes overexpressed |
| 14 | ΔyneI ΔgabD | P_{syn1}-phaC1-P_{uspA}-sucD*-ssaR_{At}*; P_{rpsU}- |
| | | orfZ |
| 15 | ΔyneI ΔgabD ΔpykF | P_{syn1}-PhaC1-P_{uspA}-sucD*-ssaR_{At}*; P_{rpsU}- |
| | ΔpykA | orfZ |

The strains were grown in a 48 hour shake plate assay. The production medium consisted of 1× E2 minimal salts solution containing 30 g/L glucose and 1× Trace Salts Solution. Both E2 medium and trace elements are described in Example 2. At the end of the growth phase, the biomass and P4HB titers were determined as described in Example 1.

The results in Table 11 show that strain 15 which lacks pykF and pykA produced more P4HB than strain 14 that retained these two genes.

TABLE 11
Biomass and P4HB titer for microbial strains 14 and 15.

| Strain | Biomass Titer (g/L) | P4HB Titer (% dcw) |
|---|---|---|
| 14 | 10.26 ± 0.44 | 25.6 ± 4.8 |
| 15 | 14.17 ± 0.11 | 46.3 ± 2.2 |

### Example 5

### Improved P4HB Production by Overexpression of PEP Carboxylase

Overexpression of PEP carboxylase (FIG. 1, Reaction number 3) has been used to enhance the production of both the aspartate family of amino acids and succinate by increasing carbon flow into the TCA cycle. However, since many wild-type homologues of PEP carboxylase are feedback-regulated by L-aspartate or other TCA cycle-derived metabolites, a considerable amount of prior art has been created regarding the identification of either feedback-desensitized mutants (Sugimoto et al., U.S. Pat. No. 5,876,983; San et al., US Patent No. 2005/0170482) or alternative homologues that naturally exhibit less allosteric regulation (Rayapati and Crafton, US Patent No. 2002/0151010). To determine whether overexpression of PEP carboxylase would lead to improved P4HB titer, the following three strains were constructed using the well known biotechnology tools and methods described above. These strains contained chromosomal deletions of yneI and gabD and overexpressed a PHA synthase, a succinyl-CoA dehydrogenase, an SSA reductase, a CoA-transferase, and either wild-type PEP carboxylase (ppc_{Ec}) from E. coli (strain 17) or wild-type PEP carboxylase (ppc_{Ms}) from Medicago sativa (strain 18) which has reduced allosteric regulation (Rayapati and Crafton, US20020151010 A1). Strain 16 served as a negative control and contained only an empty vector instead of P_{syn1}-ppc_{Ec} or P_{syn1}-ppc_{Ms} (Table 12).

TABLE 12
Microbial strains used in Example 5

| | Relevant host | |
|---|---|---|
| | genome | |
| Strains | modifications | Genes overexpressed |
| 16 | ΔyneI ΔgabD | P_{syn1}-phaC1-P_{uspA}-sucD*-ssaR_{At}*; P_{rpsU}- |
| | | orfZ |
| 17 | ΔyneI ΔgabD | P_{syn1}-phaC1-P_{uspA}-sucD*-ssaR_{At}*; P_{rpsU}- |
| | | orfZ; |
| | | P_{syn1}-ppc_{Ec} |
| 18 | ΔyneI ΔgabD | P_{syn1}-phaC1-P_{uspA}-sucD*-ssaR_{At}*; P_{rpsU}- |
| | | orfZ; |
| | | P_{syn1}-ppc_{Ms}* |

The strains were grown in a 44 hour shake plate assay. The production medium consisted of 1× E2 minimal salts solution containing 25 g/L glucose and 1× Trace Salts Solution. Both E2 medium and trace elements are described in Example 2. At the end of the growth phase, the biomass and P4HB titers were determined as described in Example 1.

The results in Table 13 show that both strains 17 and 18, which express either wild-type E. coli PEP carboxylase or a less-regulated homologue thereof, produced significantly higher amounts of P4HB than control strain 16.

TABLE 13
Biomass and P4HB titer for microbial strains 16, 17 and 18.

| Strains | Biomass Titer (g/L) | P4HB Titer (% dcw) |
|---|---|---|
| 16 | 2.31 ± 0.01 | 14.93 ± 0.83 |
| 17 | 2.85 ± 0.29 | 25.57 ± 1.59 |
| 18 | 3.02 ± 0.13 | 24.31 ± 0.65 |

### Example 6

### Improved P4HB Production by Deleting Malic Enzymes

E. coli possesses two isoforms of malic enzyme which require either NAD (maeA) or NADP⁺ (maeB) as reducing cofactor (Bologna et al., J. Bacteria 189(16):5937-5946 (2007) for the reversible conversion of malate to pyruvate (FIG. 1, Reaction number 4). Deletion of both maeA and maeB has been shown to enhance the production of L-lysine and L-threonine in E. coli, presumably by preventing the loss of carbon from the TCA cycle (van Dien et al., WO Patent No. 2005/010175). To determine if deleting both malic enzymes would also lead to improved P4HB titers, the following two strains were constructed using the well known biotechnology tools and methods described above. Both of these strains contained chromosomal deletions of yneI and gabD and overexpressed a PHA synthase, a succinyl-CoA dehydrogenase, an SSA reductase and a CoA-transferase. Strain 19 retained its native unmodified copies of maeA and maeB on the chromosome, while strain 20 has both of these genes removed (Table 14).

TABLE 14
Microbial strains used in Example 6

| | Relevant host genome | |
|---|---|---|
| Strains | modifications | Genes overexpressed |
| 19 | ΔyneI ΔgabD | P_{syn1}-phaC1-P_{uspA}-sucD*-ssaR_{At}*; |
| | | P_{rpsU}-orfZ |
| 20 | ΔyneI ΔgabD ΔmaeA | P_{syn1}-phaC1-P_{uspA}-sucD*-ssaR_{At}*; |
| | ΔmaeB | P_{rpsU}-orfZ |

The strains were grown in a 48 hour shake plate assay. The production medium consisted of 1× E2 minimal salts solution containing 30 g/L glucose and 1× Trace Salts Solution. Both E2 medium and trace elements are described in Example 2. At the end of the growth phase, the biomass and P4HB titers were determined as described in Example 1.

The results in Table 15 show that strain 20 which lacks maeA and maeB produced more P4HB than strain 19 which retained these two genes.

TABLE 15
Biomass and P4HB titer for microbial strains 19 and 20

| Strain | Biomass Titer (g/L) | P4HB Titer (% dcw) |
|---|---|---|
| 19 | 10.26 ± 0.44 | 25.6 ± 4.8 |
| 20 | 12.50 ± 1.15 | 40.0 ± 4.6 |

### Example 7

### Improved P4HB Production by Overexpressing the Glyoxylate Bypass

### Effect of Removing the Glyoxylate Bypass Genes

Noronha et al. (Biotechnology and Bioengineering 68(3): 316-327 (2000)) concluded that the glyoxylate shunt is inactive in a fadR-positive (and iclR-positive) E. coli strain using 13C-NMR/MS. However, mutants of E. coli that are fadR-negative were described by Maloy et al. (J. Bacteriol. 143:720-725 (1980)) to have elevated levels of the glyoxylate shunt enzymes, isocitrate lyase and malate synthase. Since the LS5218 host strain parent used in these examples contains an unknown mutation in the fadR gene, called fadR601 (E. coli Genetic Resources at Yale, The Coli Genetic Stock Center, CGSC#: 6966; found at the world wide web: //cgsc.biology.yale.edu/index.php), it was of interest to investigate if carbon was channeled through the glyoxylate shunt (FIG. 1, Reaction numbers 5 and 6) and/or the oxidative branch of the TCA cycle via alpha-ketoglutarate towards succinyl-CoA. Two strains were thus constructed, both of which contained chromosomal deletions of yneI, gabD, pykF, pykA, maeA, maeB and overexpressed a PHA synthase, a succinyl-CoA dehydrogenase, an SSA reductase, a CoA-transferase and a PEP carboxylase (strain 21). Strain 22 contained additional deletions of the aceA and aceB genes encoding isocitrate lyase and malate synthase, respectively (Table 16).

TABLE 16
Microbial strains used in this section of Example 7

| | Relevant host | |
|---|---|---|
| Strains | genome modifications | Genes overexpressed |
| 21 | fadR601, ΔgabD, ΔyneI, | P_{rpsU}-orfZ, P_{syn1}-ppc_{Ec}, P_{syn1}-phaC1- |
| | ΔpykF, ΔpykA, ΔmaeA, | P_{uspA}-sucD*-ssaR_{At}* |
| | ΔmaeB | |
| 22 | fadR601, ΔgabD, ΔyneI, | P_{rpsU}-orfZ, P_{syn1}-ppc_{EC}, P_{syn1}-phaC1- |
| | ΔpykF, ΔpykA, Δmae B, | P_{uspA}-sucD*-ssaR_{At}* |
| | ΔmaeA, ΔmaeB, ΔaceB, | |
| | ΔaceA | |

The strains were grown in a 24 hour shake plate assay. The production medium consisted of 1× E2 minimal salts solution containing 15 g/L glucose, 1× Trace Salts Solution. Both E2 medium and trace elements are described in Example 2. At the end of the growth phase, the biomass and P4HB titers were determined as described in Example 1.

The results in Table 17 show that strain 22 containing an inactive glyoxylate shunt had highly reduced P4HB titers as compared to its parental strain 21.

TABLE 17
Biomass and P4HB titer for microbial strains 21 and 22

| Strains | Biomass Titer (g/L) | P4HB Titer (% dcw) |
|---|---|---|
| 21 | 3.5 ± 0.3 | 20.2 ± 7.0 |
| 22 | 3.0 ± 0.1 | 7.9 ± 0.3 |

### Effect of Overexpressing the Glyoxylate Bypass Genes

Two strains were constructed both of which contained chromosomal deletions of yneI, gabD, pykF, pykA and overexpressed a PHA synthase, a succinyl-CoA dehydrogenase, an SSA reductase, a CoA-synthetase and a PEP carboxylase (strain 23). Strain 24 overexpressed in addition the aceBA genes from the IPTG-inducible P_{trc} promoter while strain 23 contained an empty vector (Table 18).

TABLE 18
Microbial strains used in this section of Example 7

| | Relevant host genome | |
|---|---|---|
| Strains | modifications | Genes overexpressed |
| 23 | fadR601, ΔgabD, ΔyneI, ΔpykF, | P_{rpsU}-orfZ, P_{syn1}-ppc_{Ec}, |
| | ΔpykA | P_{syn1}-phaC1-P_{uspA}- |
| | | sucD*-ssaR_{At}* |
| 24 | fadR601, ΔgabD, ΔyneI, ΔpykF, | P_{rpsU}-orfZ, P_{syn1}-ppc_{Ec}, |
| | ΔpykA | P_{syn1}-phaC1-P_{uspA}- |
| | | sucD*-ssaR_{At}*, |
| | | P_{trc}-aceBA |

The strains were grown in a 24 hour shake plate assay. The production medium consisted of 1× E2 minimal salts solution containing 15 g/L glucose, 1× Trace Salts Solution and 100 μM IPTG. Both E2 medium and trace elements are described in Example 2. At the end of the growth phase, the biomass and P4HB titers were determined as described in Example 1.

The results in Table 19 show that strain 24 overexpressing the two glyoxylate shunt pathway enzymes produced higher P4HB titers than its parent strain 23 that did not express the aceBA genes from the P_{trc} promoter.

TABLE 19
Biomass and P4HB titer for microbial strains 23 and 24

| Strains | Biomass Titer (g/L) | P4HB Titer (% dcw) |
|---|---|---|
| 23 | 3.12 ± 0.03 | 21.0 ± 1.2 |
| 24 | 3.27 ± 0.09 | 27.0 ± 1.0 |

### Example 8

### Improved P4HB Production by Overexpressing Glyceraldehydes-3-Phosphate Dehydrogenase

Martinez et al., (Metab. Eng. 10:352-359 (2009)) genetically engineered an Escherichia coli strain to increase NADPH availability to improve the productivity of lycopene and ε-caprolactone that require NADPH in its biosynthesis. Their approach involved an alteration of the glycolysis step where glyceraldehyde-3-phosphate is oxidized to 1,3 bisphosphoglycerate. This reaction is catalyzed by NAD-dependent endogenous glyceraldehyde-3-phosphate dehydrogenase (GAPDH) encoded by the gapA gene (FIG. 1, Reaction number 1). They constructed a recombinant E. coli strain by replacing the native NAD-dependent gapA gene with a NADP-dependent GAPDH from Clostridium acetobutylicum and demonstrated significant higher lycopene and s-caprolactone productivity than the parent strains.

To determine whether the overexpression of an NADPH-generating GAPDH would lead to improved P4HB titer, the following six strains were constructed using the well known biotechnology tools and methods described earlier. All strains contained chromosomal deletions of yneI and gabD and overexpressed a PHA synthase, a succinyl-CoA dehydrogenase, an SSA reductase, a CoA-transferase. Strain 25 contained an empty vector and served as a negative control where no other recombinant gene was expressed. Strains 26 to 29 overexpressed a gene from an IPTG-inducible promoter that encodes an NADPH-generating GAPDH from various organisms, i.e. gdp1 from Kluyveromyces lactis, gap2 from Synechocystis sp. PCC6803, gapB from Bacillus subtilis, and gapN from Streptococcus pyogenes, respectively. As another control, strain 30 overexpressed the E. coli gapA gene that encodes the NADH-generating GAPDH (Table 20).

TABLE 20
Microbial strains used in Example 8

| | Relevant host | |
|---|---|---|
| | genome | |
| Strains | modifications | Genes overexpressed |
| 25 | ΔgabD, ΔyneI | P_{rpsU}-orfZ, P_{syn1}-phaC1-P_{uspA}-sucD*-ssaR_{At}* |
| 26 | ΔgabD, ΔyneI | P_{rpsU}-orfZ, P_{syn1}-phaC1-P_{uspA}-sucD*-ssaR_{At}* |
| | | P_{trc}-gdp1 |
| 27 | ΔgabD, ΔyneI | P_{rpsU}-orfZ, P_{syn1}-phaC1-P_{uspA}-sucD*-ssaR_{At}* |
| | | P_{trc}-gap2 |
| 28 | ΔgabD, ΔyneI | P_{rpsU}-orfZ, P_{syn1}-phaC1-P_{uspA}-sucD*-ssaR_{At}* |
| | | P_{trc}-gapB |
| 29 | ΔgabD, ΔyneI | P_{rpsU}-orfZ, P_{syn1}-phaC1-P_{uspA}-sucD*-ssaR_{At}* |
| | | P_{trc}-gapN |
| 30 | ΔgabD, ΔyneI | P_{rpsU}-orfZ, P_{syn1}-phaC1-P_{uspA}-sucD*-ssaR_{At}* |
| | | P_{trc}-gapA |

The strains were grown in a 24 hour shake plate assay. The production medium consisted of 1× E2 minimal salts solution containing 10 g/L glucose and 1× Trace Salts Solution and 100 μM IPTG. Both E2 medium and trace elements are described in Example 2. At the end of the growth phase, the biomass and P4HB titers were determined as described in Example 1.

The results in Table 21 show that strains 26, 27, and 29 produced higher amounts of P4HB than control strain 25. Interestingly, strain 28 produced much less P4HB than strain 25. Surprisingly, overexpression of the endogenous gapA gene encoding the NADH-generating GAPDH in strain 30 outperformed all other strains.

TABLE 21
Biomass and P4HB titer for microbial strains 25-30

| Strains | Biomass Titer (g/L) | P4HB Titer (% dcw) |
|---|---|---|
| 25 | 2.52 ± 0.03 | 14.0 ± 0.3 |
| 26 | 2.84 ± 0.01 | 25.0 ± 1.0 |
| 27 | 2.50 ± 0.10 | 21.5 ± 0.9 |
| 28 | 2.20 ± 0.10 | 2.3 ± 0.1 |
| 29 | 2.48 ± 0.01 | 21.0 ± 1.0 |
| 30 | 3.03 ± 0.08 | 32.5 ± 0.6 |

Gene ID 001 Nucleotide Sequence: Medicago sativa Phosphoenolpyruvate Carboxylase Ppc*

(SEQ ID NO. 1)
ATGGCAAACAAAATGGAAAAGATGGCAAGCATTGACGCGCAACTGCGCCAGTTGGTCCCGG
CAAAAGTCAGCGAGGACGACAAATTGATTGAATACGATGCTCTGTTGCTGGACCGCTTTCT
GGACATTCTGCAAGATCTGCATGGCGAGGATCTGAAGGATTCGGTTCAGGAAGTTTACGAA
CTGTCTGCGGAGTATGAGCGTAAGCATGACCCGAAGAAGCTGGAAGAGCTGGGTAACTTGA
TTACGAGCTTTGACGCGGGCGACAGCATTGTCGTGGCGAAATCGTTCTCTCATATGCTGAA
TCTGGCGAACCTGGCCGAAGAAGTTCAAATTGCTCACCGCCGTCGTAACAAGCTGAAGAAG
GGTGATTTTCGTGATGAGAGCAATGCGACCACCGAGTCCGATATTGAGGAGACTCTGAAGA
AACTGGTTTTCGACATGAAGAAGTCTCCGCAAGAAGTGTTTGACGCGTTGAAGAATCAGAC
CGTGGACCTGGTGCTGACGGCACATCCTACCCAGAGCGTTCGCCGTTCCCTGCTGCAAAAG
CATGGTCGTGTTCGTAATTGCTTGAGCCAGCTGTATGCGAAAGACATTACCCCGGATGACA
AACAAGAGCTGGACGAGGCACTGCAGCGTGAAATCCAGGCAGCGTTCCGTACCGATGAAAT
CAAACGTACCCCGCCGACCCCACAAGACGAAATGCGTGCTGGCATGAGCTATTTCCACGAA
ACCATCTGGAAGGGCGTCCCGAAGTTCCTGCGTCGCGTGGACACCGCGTTGAAGAACATCG
GCATTAACGAACGCGTGCCGTATAACGCCCCGCTGATTCAATTCAGCAGCTGGATGGGTGG
CGACCGTGACGGCAATCCGCGTGTTACGCCAGAAGTGACCCGTGATGTTTGTCTGCTGGCG
CGTATGATGGCGGCGAATTTGTACTATAGCCAGATTGAAGATCTGATGTTTGAGCTGTCTA
TGTGGCGCTGTAATGATGAGTTGCGTGTGCGTGCCGAAGAACTGCACCGCAATAGCAAGAA
AGACGAAGTTGCCAAGCACTACATCGAGTTCTGGAAGAAGATCCCGTTGAACGAGCCGTAC
CGTGTTGTTCTGGGTGAGGTCCGCGATAAGCTGTATCGCACCCGTGAGCGCAGCCGTTATC
TGCTGGCACACGGTTATTGCGAAATTCCGGAGGAGGCGACCTTTACCAACGTGGATGAATT
TCTGGAACCGCTGGAGCTGTGTTATCGTAGCCTGTGCGCGTGCGGTGACCGCGCGATTGCG
GACGGTTCTTTGCTGGATTTCCTGCGCCAGGTGAGCACGTTTGGTCTGAGCCTGGTCCGTC
TGGATATCCGTCAGGAATCGGACCGCCATACGGATGTGATGGACGCTATTACCAAACACCT
GGAAATTGGCAGCTACCAGGAGTGGAGCGAGGAGAAACGTCAAGAGTGGCTGCTGAGCGAG
CTGATCGGTAAGCGTCCGCTGTTCGGTCCAGATCTGCCGCAAACCGACGAAATCCGCGACG
TTCTGGACACCTTTCGTGTGATTGCCGAACTGCCGAGCGACAACTTCGGCGCGTACATTAT
CTCCATGGCCACCGCCCCGAGCGATGTCCTGGCAGTCGAGCTGCTGCAACGCGAATGTAAG
GTCCGTAACCCGTTGCGCGTGGTTCCGCTGTTTGAAAAGCTGGATGACCTGGAGAGCGCAC
CGGCCGCACTGGCTCGTCTGTTTAGCATTGACTGGTACATTAACCGTATTGATGGTAAACA
GGAAGTGATGATTGGTTACTCCGACAGCGGTAAAGATGCGGGTCGTTTTAGCGCCGCATGG
CAGCTGTACAAGGCACAAGAAGATCTGATCAAGGTTGCACAGAAGTTCGGCGTTAAACTGA
CCATGTTCCACGGTCGCGGTGGTACGGTTGGCCGTGGTGGCGGCCCAACCCACCTGGCGAT
TCTGAGCCAACCGCCGGAGACTATCCATGGTTCCTTGCGTGTCACCGTCCAGGGCGAAGTG
ATTGAGCAAAGCTTCGGCGAGGAACATCTGTGCTTTCGCACCCTGCAGCGTTTTACGGCCG
CGACTTTGGAACACGGCATGCGTCCGCCATCCAGCCCAAAGCCAGAATGGCGTGCGCTGAT
GGACCAAATGGCGGTTATCGCGACCGAGGAGTATCGCAGCATTGTGTTCAAAGAGCCGCGT
TTTGTGGAGTATTTCCGTTTGGCAACGCCGGAGATGGAGTACGGCCGCATGAATATCGGCA
GCCGTCCGGCAAAACGTCGCCCGTCCGGCGGCATCGAGACGCTGCGTGCCATCCCGTGGAT
TTTCGCGTGGACGCAGACCCGTTTCCATTTGCCGGTGTGGCTGGGTTTCGGTGCCGCCTTT
CGTCAAGTCGTGCAGAAGGACGTGAAGAATCTGCATATGCTGCAGGAGATGTACAACCAGT
GGCCGTTCTTTCGTGTCACCATTGATCTGGTGGAAATGGTCTTTGCGAAAGGTGATCCGGG
CATCGCGGCGTTGAATGACCGTCTGCTGGTTTCCAAAGACCTGTGGCCTTTTGGTGAACAG
CTGCGTAGCAAGTACGAGGAAACCAAGAAACTGCTGTTGCAAGTTGCGGCGCACAAGGAGG
TGCTGGAAGGTGACCCTTATCTGAAGCAACGCCTGCGTCTGCGTGACTCGTACATCACGAC
CCTGAATGTCTTTCAGGCGTATACCCTGAAGCGTATCCGTGACCCGAATTACAAAGTGGAA
GTTCGCCCTCCGATCAGCAAGGAGAGCGCGGAGACTAGCAAACCAGCGGACGAACTGGTCA
CCCTGAATCCGACCTCGGAGTATGCTCCGGGTTTGGAAGATACGCTGATTCTGACGATGAA
GGGTATCGCGGCTGGCATGCAGAACACGGGCTAA

Gene ID 001 Protein Sequence: Medicago sativa Phosphoenolpyruvate Carboxylase Ppc*

(SEQ ID NO. 2)
MANKMEKMASIDAQLRQLVPAKVSEDDKLIEYDALLLDRFLDILQDLHGEDLKDSVQEVYE
LSAEYERKHDPKKLEELGNLITSFDAGDSIVVAKSFSHMLNLANLAEEVQIAHRRRNKLKK
GDFRDESNATTESDIEETLKKLVFDMKKSPQEVFDALKNQTVDLVLTAHPTQSVRRSLLQK
HGRVRNCLSQLYAKDITPDDKQELDEALQREIQAAFRTDEIKRTPPTPQDEMRAGMSYFHE
TIWKGVPKFLRRVDTALKNIGINERVPYNAPLIQFSSWMGGDRDGNPRVTPEVTRDVCLLA
RMMAANLYYSQIEDLMFELSMWRCNDELRVRAEELHRNSKKDEVAKHYIEFWKKIPLNEPY
RVVLGEVRDKLYRTRERSRYLLAHGYCEIPEEATFTNVDEFLEPLELCYRSLCACGDRAIA
DGSLLDFLRQVSTFGLSLVRLDIRQESDRHTDVMDAITKHLEIGSYQEWSEEKRQEWLLSE
LIGKRPLFGPDLPQTDEIRDVLDTFRVIAELPSDNFGAYIISMATAPSDVLAVELLQRECK
VRNPLRVVPLFEKLDDLESAPAALARLFSIDWYINRIDGKQEVMIGYSDSGKDAGRFSAAW
QLYKAQEDLIKVAQKFGVKLTMFHGRGGTVGRGGGPTHLAILSQPPETIHGSLRVTVQGEV
IEQSFGEEHLCFRTLQRFTAATLEHGMRPPSSPKPEWRALMDQMAVIATEEYRSIVFKEPR
FVEYFRLATPEMEYGRMNIGSRPAKRRPSGGIETLRAIPWIFAWTQTRFHLPVWLGFGAAF
RQVVQKDVKNLHMLQEMYNQWPFFRVTIDLVEMVFAKGDPGIAALNDRLLVSKDLWPFGEQ
LRSKYEETKKLLLQVAAHKEVLEGDPYLKQRLRLRDSYITTLNVFQAYTLKRIRDPNYKVE
VRPPISKESAETSKPADELVTLNPTSEYAPGLEDTLILTMKGIAAGMQNTG

Gene ID 002 Nucleotide Sequence: Clostridium kluyveri Succinate Semialdehyde Dehydrogenase sucD*

(SEQ ID NO. 3)
ATGTCCAACGAGGTTAGCATTAAGGAGCTGATTGAGAAGGCGAAAGTGGCGCAGAAAAAGC
TGGAAGCGTATAGCCAAGAGCAAGTTGACGTTCTGGTCAAGGCGCTGGGTAAAGTTGTGTA
CGACAACGCCGAGATGTTCGCGAAAGAGGCGGTGGAGGAAACCGAGATGGGTGTTTACGAG
GATAAAGTGGCTAAATGTCATCTGAAATCTGGTGCAATCTGGAATCACATTAAAGATAAGA
AAACCGTTGGTATTATCAAGGAAGAACCGGAGCGTGCGCTGGTGTACGTCGCGAAGCCTAA
AGGTGTTGTGGCGGCGACGACCCCTATCACCAATCCTGTGGTTACCCCGATGTGTAACGCG
ATGGCAGCAATTAAAGGTCGCAACACCATCATTGTCGCCCCGCATCCGAAGGCGAAGAAGG
TGAGCGCGCACACCGTGGAGCTGATGAATGCAGAACTGAAAAAGTTGGGTGCGCCGGAAAA
CATTATCCAGATCGTTGAAGCCCCAAGCCGTGAAGCAGCCAAGGAGTTGATGGAGAGCGCA
GACGTGGTTATCGCCACGGGTGGCGCAGGCCGTGTTAAAGCAGCGTACTCCTCCGGCCGTC
CGGCATACGGTGTCGGTCCGGGCAATTCTCAGGTCATTGTCGATAAGGGTTACGATTATAA
CAAAGCTGCCCAGGACATCATTACCGGCCGCAAGTATGACAACGGTATCATTTGCAGCTCT
GAGCAGAGCGTGATCGCACCGGCGGAGGACTACGACAAGGTCATCGCGGCTTTCGTCGAGA
ATGGCGCGTTCTATGTCGAGGATGAGGAAACTGTGGAGAAATTCCGTAGCACGCTGTTCAA
GGATGGCAAGATCAATAGCAAAATCATCGGTAAATCCGTGCAGATCATCGCTGACCTGGCT
GGTGTCAAGGTGCCGGAAGGCACCAAGGTGATCGTGTTGAAGGGCAAGGGTGCCGGTGAAA
AGGACGTTCTGTGCAAGGAGAAAATGTGCCCGGTCCTGGTTGCCCTGAAATATGACACCTT
TGAGGAGGCGGTCGAGATCGCGATGGCCAACTATATGTACGAGGGTGCGGGCCATACCGCC
GGTATCCACAGCGATAACGACGAGAATATCCGCTACGCGGGTACGGTGCTGCCAATCAGCC
GTCTGGTTGTCAACCAGCCAGCAACTACGGCCGGTGGTAGCTTTAACAATGGTTTTAATCC
GACCACCACCTTGGGCTGCGGTAGCTGGGGCCGTAACTCCATTAGCGAGAACCTGACGTAT
GAGCATCTGATTAATGTCAGCCGTATTGGCTATTTCAATAAGGAGGCAAAAGTTCCTAGCT
ACGAGGAGATCTGGGGTTAA

Gene ID 002 Protein Sequence: Clostridium kluyveri Succinate Semialdehyde Dehydrogenase sucD*

(SEQ ID NO. 4)
MSNEVSIKELIEKAKVAQKKLEAYSQEQVDVLVKALGKVVYDNAEMFAKEAVEETEMGVYE
DKVAKCHLKSGAIWNHIKDKKTVGIIKEEPERALVYVAKPKGVVAATTPITNPVVTPMCNA
MAAIKGRNTIIVAPHPKAKKVSAHTVELMNAELKKLGAPENIIQIVEAPSREAAKELMESA
DVVIATGGAGRVKAAYSSGRPAYGVGPGNSQVIVDKGYDYNKAAQDIITGRKYDNGIICSS
EQSVIAPAEDYDKVIAAFVENGAFYVEDEETVEKFRSTLFKDGKINSKIIGKSVQIIADLA
GVKVPEGTKVIVLKGKGAGEKDVLCKEKMCPVLVALKYDTFEEAVEIAMANYMYEGAGHTA
GIHSDNDENIRYAGTVLPISRLVVNQPATTAGGSFNNGFNPTTTLGCGSWGRNSISENLTY
EHLINVSRIGYFNKEAKVPSYEEIWG

Gene ID 003 Nucleotide Sequence: Arabidopsis thaliana Succinic Semialdehyde Reductase ssaR_{At}*

(SEQ ID NO. 5)
ATGGAAGTAGGTTTTCTGGGTCTGGGCATTATGGGTAAAGCTATGTCCATGAACCTGCTGA
AAAACGGTTTCAAAGTTACCGTGTGGAACCGCACTCTGTCTAAATGTGATGAACTGGTTGA
ACACGGTGCAAGCGTGTGCGAGTCTCCGGCTGAGGTGATCAAGAAATGCAAATACACGATC
GCGATGCTGAGCGATCCGTGTGCAGCTCTGTCTGTTGTTTTCGATAAAGGCGGTGTTCTGG
AACAGATCTGCGAGGGTAAGGGCTACATCGACATGTCTACCGTCGACGCGGAAACTAGCCT
GAAAATTAACGAAGCGATCACGGGCAAAGGTGGCCGTTTTGTAGAAGGTCCTGTTAGCGGT
TCCAAAAAGCCGGCAGAAGACGGCCAGCTGATCATCCTGGCAGCAGGCGACAAAGCACTGT
TCGAGGAATCCATCCCGGCCTTTGATGTACTGGGCAAACGTTCCTTTTATCTGGGTCAGGT
GGGTAACGGTGCGAAAATGAAACTGATTGTTAACATGATCATGGGTTCTATGATGAACGCG
TTTAGCGAAGGTCTGGTACTGGCAGATAAAAGCGGTCTGTCTAGCGACACGCTGCTGGATA
TTCTGGATCTGGGTGCTATGACGAATCCGATGTTCAAAGGCAAAGGTCCGTCCATGACTAA
ATCCAGCTACCCACCGGCTTTCCCGCTGAAACACCAGCAGAAAGACATGCGTCTGGCTCTG
GCTCTGGGCGACGAAAACGCTGTTAGCATGCCGGTCGCTGCGGCTGCGAACGAAGCCTTCA
AGAAAGCCCGTAGCCTGGGCCTGGGCGATCTGGACTTTTCTGCTGTTATCGAAGCGGTAAA
ATTCTCTCGTGAATAA

Gene ID 003 Protein Sequence: Arabidopsis thaliana Succinic Semialdehyde Reductase ssaR_{At}*

(SEQ ID NO. 6)
MEVGFLGLGIMGKAMSMNLLKNGFKVTVWNRTLSKCDELVEHGASVCESPAEVIKKCKYTI
AMLSDPCAALSVVFDKGGVLEQICEGKGYIDMSTVDAETSLKINEAITGKGGRFVEGPVSG
SKKPAEDGQLIILAAGDKALFEESIPAFDVLGKRSFYLGQVGNGAKMKLIVNMIMGSMMNA
FSEGLVLADKSGLSSDTLLDILDLGAMTNPMFKGKGPSMTKSSYPPAFPLKHQQKDMRLAL
ALGDENAVSMPVAAAANEAFKKARSLGLGDLDFSAVIEAVKFSRE

Gene ID 004 Nucleotide Sequence: Aspergillus terreus Succinic Semialdehyde Reductase ssaR_{At2}*

(SEQ ID NO. 7)
ATGCCACTGGTTGCTCAAAATCCACTGCCACGTGCTATTCTGGGTCTGATGACTTTCGGTC
CGAGCGAAAGCAAAGGTGCGCGTATCACTTCCCTGGATGAGTTTAACAAGTGCCTGGATTA
CTTCCAGCAGCAGGGCTTCCAGGAAATCGATACCGCGCGCATCTACGTCGGCGGTGAACAG
GAGGCATTCACGGCGCAGGCAAAGTGGAAAGAACGCGGCCTGACGCTGGCGACTAAGTGGT
ATCCGCAGTACCCGGGTGCGCACAAACCGGATGTCCTGCGTCAGAACCTGGAGCTGTCCCT
GAAAGAACTGGGCACGAACCAGGTCGATATCTTCTATCTGCACGCCGCGGATCGTTCTGTG
CCGTTCGCGGAAACTCTGGAAACTGTTAACGAACTGCACAAAGAAGGCAAATTTGTTCAGC
TGGGTCTGTCTAACTACACCGCTTTCGAAGTAGCTGAAATCGTGACCCTGTGTAACGAGCG
TGGTTGGGTTCGTCCGACTATCTACCAGGCGATGTATAACGCTATCACCCGTAACATCGAA
ACTGAACTGATCCCGGCGTGCAAGCGTTACGGTATTGACATTGTTATCTACAACCCACTGG
CGGGTGGCCTGTTCAGCGGCAAATACAAAGCACAGGACATCCCGGCTGAAGGTCGTTACAG
CGACCAATCTTCCATGGGCCAGATGTACCGCAACCGTTACTTTAAGGACGCAACCTTTGAC
GCTCTGCGCCTGATCGAACCGGTTGTTGCGAAGCACGGCCTGACGATGCCGGAAACCGCGT
TCCGCTGGGTCCACCACCACTCCGCACTGAACATGGAAGATGGCGGCCGTGACGGCATCAT
TCTGGGTGTAAGCAGCCTGGCTCAGCTGGAAAACAACCTGAAAGACATTCAGAAAGGTCCG
CTGCCGCAGGAGGTTGTAGACGTCCTGGATCAGGCTTGGCTGGTGGCTAAGCCGACGGCTC
CAAACTACTGGCATCTGGACCTGAAATACACGTACGACACCCAGGAAGCTCTGTTCAAACC
GAAATCTAAGGCGTAA

Gene ID 004 Protein Sequence: Aspergillus terreus Succinic Semialdehyde Reductase ssaR_{At2}*

(SEQ ID NO. 8)
MPLVAQNPLPRAILGLMTFGPSESKGARITSLDEFNKCLDYFQQQGFQEIDTARIYVGGEQ
EAFTAQAKWKERGLTLATKWYPQYPGAHKPDVLRQNLELSLKELGTNQVDIFYLHAADRSV
PFAETLETVNELHKEGKFVQLGLSNYTAFEVAEIVTLCNERGWVRPTIYQAMYNAITRNIE
TELIPACKRYGIDIVIYNPLAGGLFSGKYKAQDIPAEGRYSDQSSMGQMYRNRYFKDATFD
ALRLIEPVVAKHGLTMPETAFRWVHHHSALNMEDGGRDGIILGVSSLAQLENNLKDIQKGP
LPQEVVDVLDQAWLVAKPTAPNYWHLDLKYTYDTQEALFKPKSKAAVKFSRE

Gene ID 005 Nucleotide Sequence: Mus musculus Succinic Semialdehyde Reductase ssaR_{Mm}*

(SEQ ID NO. 9)
ATGCTGCGTGCTGCTTCTCGTGCTGTTGGTCGTGCTGCTGTACGTTCCGCTCAACGTTCTG
GTACTAGCGTTGGCCGTCCGCTGGCGATGTCCCGTCCACCGCCGCCTCGCGCAGCTAGCGG
TGCCCCGCTGCGTCCGGCAACCGTACTGGGCACTATGGAGATGGGTCGTCGCATGGACGCT
TCTGCATCCGCGGCAAGCGTTCGTGCGTTCCTGGAACGTGGCCATAGCGAACTGGATACCG
CTTTCATGTATTGCGACGGTCAGTCCGAAAATATCCTGGGTGGCCTGGGCCTGGGTCTGGG
CTCCGGTGATTGTACCGTTAAAATTGCGACCAAGGCGAACCCTTGGGAGGGCAAGAGCCTG
AAGCCGGATTCTGTGCGTTCTCAGCTGGAGACTTCTCTGAAACGTCTGCAGTGTCCGCGCG
TAGACCTGTTCTATCTGCATGCGCCGGACCACAGCACTCCGGTAGAGGAAACTCTGCGTGC
GTGTCATCAGCTGCACCAGGAAGGCAAGTTCGTCGAACTGGGTCTGTCTAACTACGCATCT
TGGGAAGTGGCAGAAATCTGTACGCTGTGTAAGTCTAATGGTTGGATCCTGCCAACCGTGT
ACCAGGGCATGTACAACGCTACCACCCGCCAGGTAGAAGCAGAACTGCTGCCGTGCCTGCG
TCACTTCGGCCTGCGCTTTTACGCTTACAACCCGCTGGCGGGTGGTCTGCTGACGGGCAAA
TACAAGTATGAAGATAAAGATGGTAAACAACCGGTCGGTCGTTTCTTTGGTAACAACTGGG
CCGAAACCTACCGTAATCGCTTCTGGAAAGAGCACCACTTTGAAGCGATCGCACTGGTTGA
AAAAGCGCTGCAGACGACTTATGGCACTAACGCGCCGCGTATGACCTCCGCTGCGCTGCGT
TGGATGTACCACCATAGCCAGCTGCAGGGTACTCGCGGCGATGCCGTTATCCTGGGCATGA
GCTCCCTGGAACAGCTGGAACAGAACCTGGCCGCGACTGAAGAGGGCCCGCTGGAACCGGC
AGTTGTCGAAGCTTTTGACCAGGCATGGAACATGGTGGCGCACGAATGTCCAAACTATTTC
CGCTAA

Gene ID 005 Protein Sequence: Mus musculus Succinic Semialdehyde Reductase ssaR_{Mm}*

(SEQ ID NO. 10)
MLRAASRAVGRAAVRSAQRSGTSVGRPLAMSRPPPPRAASGAPLRPATVLGTMEMGRRMDA
SASAASVRAFLERGHSELDTAFMYCDGQSENILGGLGLGLGSGDCTVKIATKANPWEGKSL
KPDSVRSQLETSLKRLQCPRVDLFYLHAPDHSTPVEETLRACHQLHQEGKFVELGLSNYAS
WEVAEICTLCKSNGWILPTVYQGMYNATTRQVEAELLPCLRHFGLRFYAYNPLAGGLLTGK
YKYEDKDGKQPVGRFFGNNWAETYRNRFWKEHHFEAIALVEKALQTTYGTNAPRMTSAALR
WMYHHSQLQGTRGDAVILGMSSLEQLEQNLAATEEGPLEPAVVEAFDQAWNMVAHECPNYF
R

Gene ID 006 Nucleotide Sequence: Pseudomonas putida/Ralstonia Eutropha JMP134 Polyhydroxyalkanoate Synthase Fusion Protein phaC3/C1

(SEQ ID NO. 11)
ATGACTAGAAGGAGGTTTCATATGAGTAACAAGAACAACGATGAGCTGGCGACGGGTAAAG
GTGCTGCTGCATCTTCTACTGAAGGTAAATCTCAGCCGTTTAAATTCCCACCGGGTCCGCT
GGACCCGGCCACTTGGCTGGAATGGAGCCGTCAGTGGCAAGGTCCGGAGGGCAATGGCGGT
ACCGTGCCGGGTGGCTTTCCGGGTTTCGAAGCGTTCGCGGCGTCCCCGCTGGCGGGCGTGA
AAATCGACCCGGCTCAGCTGGCAGAGATCCAGCAGCGTTATATGCGTGATTTCACCGAGCT
GTGGCGTGGTCTGGCAGGCGGTGACACCGAGAGCGCTGGCAAACTGCATGACCGTCGCTTC
GCGTCCGAAGCGTGGCACAAAAACGCGCCGTATCGCTATACTGCGGCATTTTACCTGCTGA
ACGCACGTGCACTGACGGAACTGGCTGATGCAGTAGAAGCGGATCCGAAAACCCGTCAGCG
TATCCGTTTTGCGGTTTCCCAGTGGGTAGATGCTATGAGCCCGGCTAACTTCCTGGCCACC
AACCCGGACGCTCAGAACCGTCTGATCGAGAGCCGTGGTGAAAGCCTGCGTGCCGGCATGC
GCAATATGCTGGAAGATCTGACCCGCGGTAAAATTTCCCAAACCGATGAGACTGCCTTCGA
AGTAGGCCGTAACATGGCAGTTACCGAAGGTGCTGTGGTATTCGAAAACGAGTTCTTCCAG
CTGCTGCAGTACAAACCTCTGACTGACAAAGTATACACCCGTCCGCTGCTGCTGGTACCGC
CGTGCATTAACAAGTTCTATATTCTGGACCTGCAGCCGGAAGGTTCTCTGGTCCGTTACGC
AGTCGAACAGGGTCACACTGTATTCCTGGTGAGCTGGCGCAATCCAGACGCTAGCATGGCT
GGCTGTACCTGGGATGACTATATTGAAAACGCGGCTATCCGCGCCATCGAGGTTGTGCGTG
ATATCAGCGGTCAGGACAAGATCAACACCCTGGGCTTTTGTGTTGGTGGCACGATCATCTC
CACTGCCCTGGCGGTCCTGGCCGCCCGTGGTGAGCACCCGGTGGCCTCTCTGACCCTGCTG
ACTACCCTGCTGGACTTCACCGATACTGGTATCCTGGATGTTTTCGTGGACGAGCCACACG
TTCAGCTGCGTGAGGCGACTCTGGGCGGCGCCAGCGGCGGTCTGCTGCGTGGTGTCGAGCT
GGCCAATACCTTTTCCTTCCTGCGCCCGAACGACCTGGTTTGGAACTACGTTGTTGACAAC
TATCTGAAAGGCAACACCCCGGTACCTTTCGATCTGCTGTTCTGGAACGGTGATGCAACCA
ACCTGCCTGGTCCATGGTACTGTTGGTACCTGCGTCATACTTACCTGCAGAACGAACTGAA
AGAGCCGGGCAAACTGACCGTGTGTAACGAACCTGTGGACCTGGGCGCGATTAACGTTCCT
ACTTACATCTACGGTTCCCGTGAAGATCACATCGTACCGTGGACCGCGGCTTACGCCAGCA
CCGCGCTGCTGAAGAACGATCTGCGTTTCGTACTGGGCGCATCCGGCCATATCGCAGGTGT
GATCAACCCTCCTGCAAAGAAAAAGCGTTCTCATTGGACCAACGACGCGCTGCCAGAATCC
GCGCAGGATTGGCTGGCAGGTGCTGAGGAACACCATGGTTCCTGGTGGCCGGATTGGATGA
CCTGGCTGGGTAAACAAGCCGGTGCAAAACGTGCAGCTCCAACTGAATATGGTAGCAAGCG
TTATGCTGCAATCGAGCCAGCGCCAGGCCGTTACGTTAAAGCGAAAGCATAA

Gene ID 006 Protein Sequence: Pseudomonas putida/Ralstonia Eutropha JMP134 Polyhydroxyalkanoate Synthase Fusion Protein phaC3/C1

(SEQ ID NO. 12)
MSNKNNDELATGKGAAASSTEGKSQPFKFPPGPLDPATWLEWSRQWQGPEGNGGTVPGGFP
GFEAFAASPLAGVKIDPAQLAEIQQRYMRDFTELWRGLAGGDTESAGKLHDRRFASEAWHK
NAPYRYTAAFYLLNARALTELADAVEADPKTRQRIRFAVSQWVDAMSPANFLATNPDAQNR
LIESRGESLRAGMRNMLEDLTRGKISQTDETAFEVGRNMAVTEGAVVFENEFFQLLQYKPL
TDKVYTRPLLLVPPCINKFYILDLQPEGSLVRYAVEQGHTVFLVSWRNPDASMAGCTWDDY
IENAAIRAIEVVRDISGQDKINTLGFCVGGTIISTALAVLAARGEHPVASLTLLTTLLDFT
DTGILDVFVDEPHVQLREATLGGASGGLLRGVELANTFSFLRPNDLVWNYVVDNYLKGNTP
VPFDLLFWNGDATNLPGPWYCWYLRHTYLQNELKEPGKLTVCNEPVDLGAINVPTYIYGSR
EDHIVPWTAAYASTALLKNDLRFVLGASGHIAGVINPPAKKKRSHWTNDALPESAQDWLAG
AEEHHGSWWPDWMTWLGKQAGAKRAAPTEYGSKRYAAIEPAPGRYVKAKA

### Example 9

### Generation of Gamma-Butyrolactone from the Pyrolysis of a Genetically Engineered Microbe Producing Poly-4-hydroxybutyrate

Biomass containing poly(4-hydroxybutyrate) (P4HB) was produced in a 20 L New Brunswick Scientific fermentor (BioFlo 4500) using a genetically modified E. coli strain specifically designed for production of poly-4HB from glucose syrup as a carbon feed source. Examples of the E. coli strains, fermentation conditions, media and feed conditions are described in U.S. Pat. Nos. 6,316,262; 6,689,589; 7,081,357; and 7,229,804 incorporated by reference herein. The E. coli strain generated a fermentation broth which had a P4HB titer of approximately 100-120 g of P4HB/kg of broth. After the fermentation was complete, 100 g of the fermentation broth (e.g. P4HB biomass) was mixed with an aqueous slurry containing 10% by weight lime (Ca(OH)₂ 95+%, Sigma Aldrich). A 2 g portion of the broth+lime mixture was then dried in an aluminum weigh pan at 150° C. using an infrared heat balance (MB-45 Ohaus Moisture Analyzer) to constant weight. Residual water remaining was <5% by weight. The final lime concentration in the dry broth was 50 g lime/kg of dry solids or 5% by wt. A sample containing only dried fermentation broth (no lime addition) was prepared as well. Additionally, a sample of pure poly-4HB was recovered by solvent extraction as described in U.S. Pat. Nos. 7,252,980 and 7,713,720, followed by oven drying to remove the residual solvent.

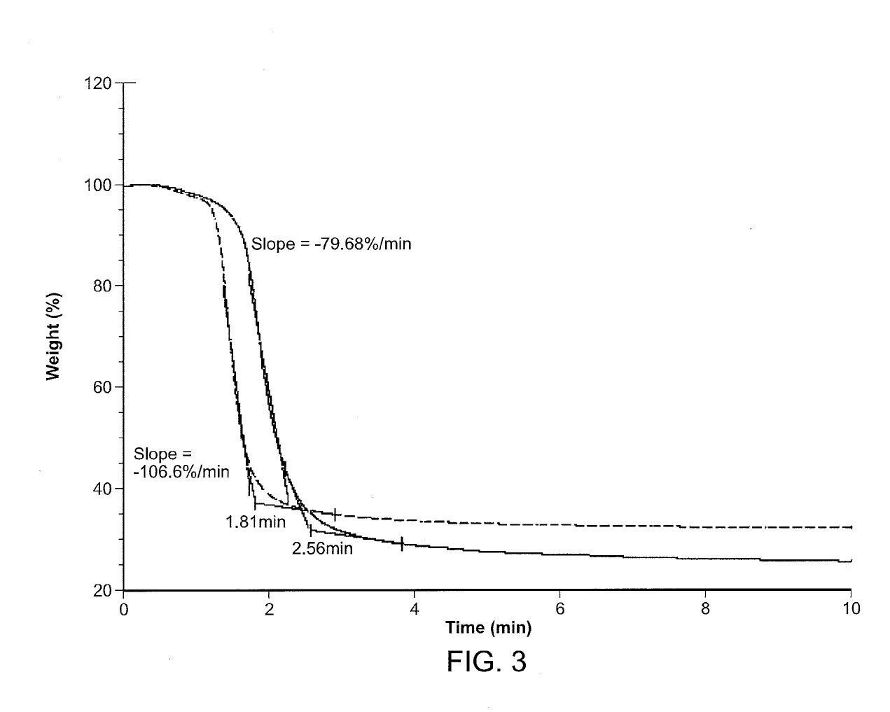

The dry P4HB biomass samples were analyzed by TGA using an isothermal temperature of 300° C. under a N₂ gas purge. FIG. 3 shows the TGA weight loss vs. time curves for the dry fermentation broth with lime (dashed curve), and without lime (solid curve). Each dry broth sample showed a single major weight loss event. Also shown in the plots are the slopes of the weight loss curves (indicating the thermal degradation rate) and the onset times for completion of weight loss. Table 22 shows the thermal degradation rate data for the two dry broth samples. With the addition of 5 wt % lime, the dry broth showed a 34% faster rate of weight loss as compared to the dry broth with no lime added. Also the onset time for completion of thermal degradation was approximately 30% shorter in the dry broth with added lime sample. These results showed that the lime catalyst significantly sped up the P4HB biomass thermal degradation process.

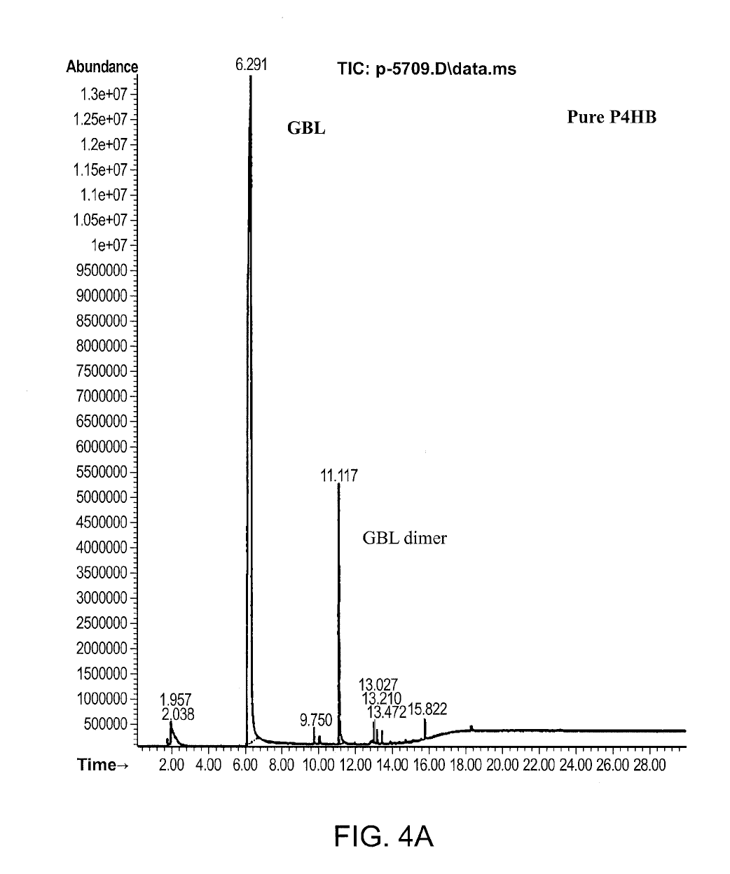

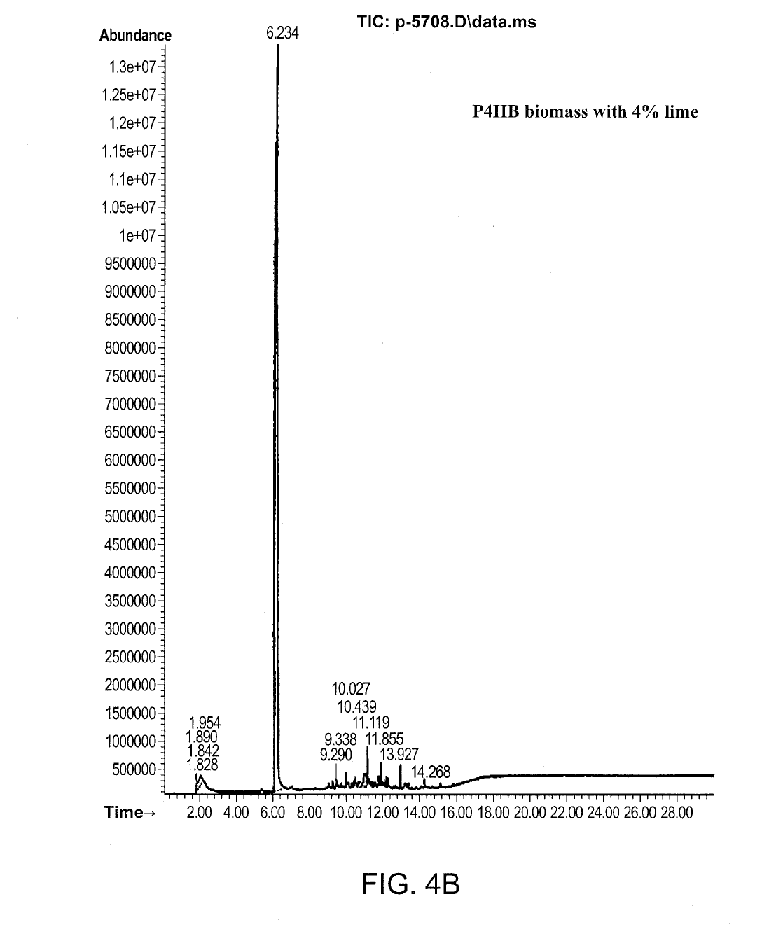

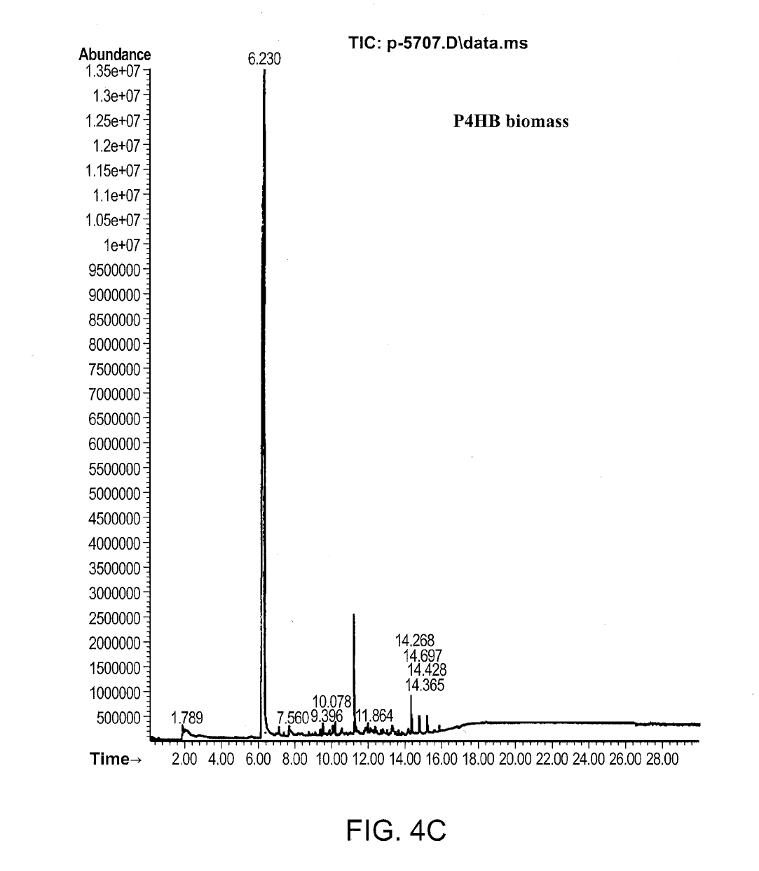

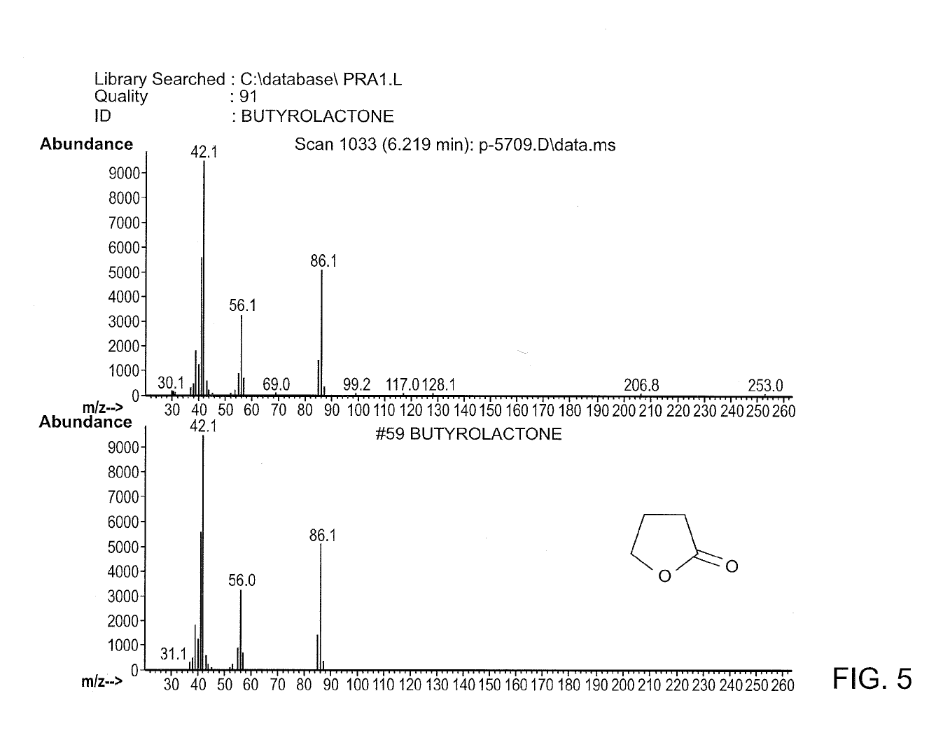

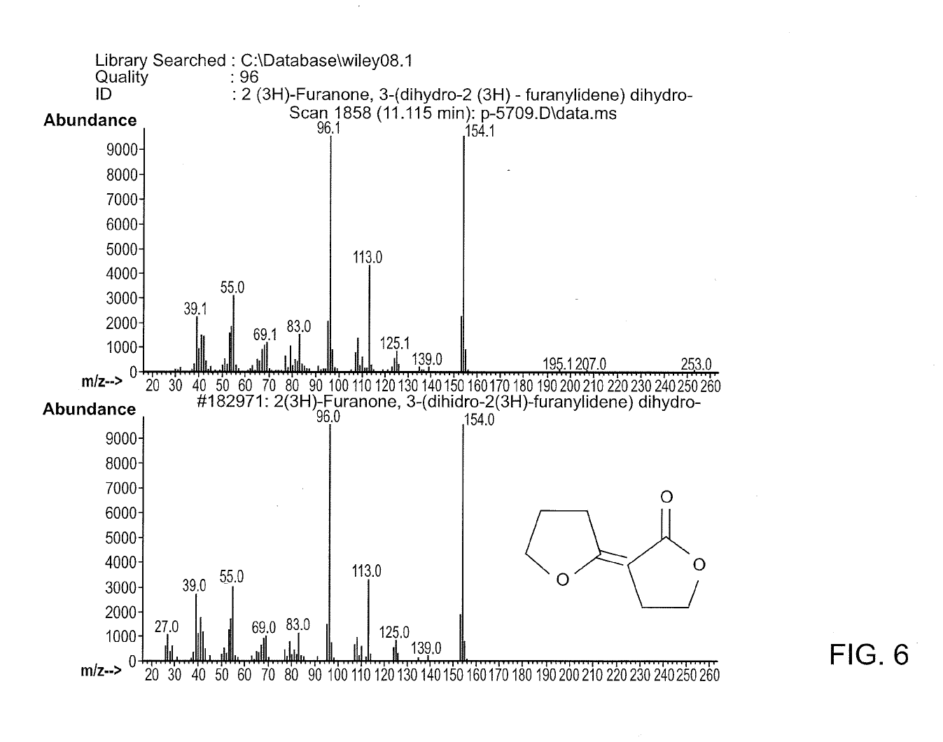

Both dry broth samples and a pure poly-4HB sample were then analyzed by Py-GC-MS in order to identify the compounds being generated during thermal degradation at 300° C. in an inert atmosphere. FIG. 4 shows the chromatograms of pyrolyzed pure poly-4HB, dry broth without added lime, and dry broth with added lime. For all of the samples, two major thermal degradation components were identified from the pyrolysis at 300° C.: GBL (peak at 6.2 min), and the dimer of GBL (peak at 11.1 min). The dimer of GBL was identified as (3-(dihydro-2(3H)-furanylidene)dihydro-2(3H)-furanone). FIG. 4 shows the mass spectral library matches identifying these two peaks.

Table 22 below summarizes the Py-GC-MS data measured for the pure poly-4HB polymer, dry poly-4HB broth without added lime, and the dry poly-4HB broth with added lime. Both the selectivity and yield of GBL from broth were observed to increase with addition of the lime catalyst. The yield was calculated by taking the GBL peak area counts and dividing by the weight of P4HB in each sample. For the broth samples, the % P4HB was measured to be ˜49% by weight of the total biomass. The fermentation broth media typically has potassium (4-7% by wt.) and sodium metal salts (<1% by wt.) present in it so that the increase in the yield of GBL was only 10% after lime addition. However, the selectivity for GBL was increased by a factor of 2 after the lime addition. As is evident from Table 22, higher lime concentration suppressed the formation of the GBL dimer, while increasing the yield of GBL relative to weight of poly-4HB pyrolyzed.

TABLE 22
Summary of Pyrolysis-GC-MS at 300° C. and TGA data for
poly-4HB pure polymer, dry poly-4HB broth and dry poly-4HB
broth with added lime.

| | | Area Counts | Thermal |
|---|---|---|---|
| | Ratio of | GBL/mg of | Degradation |
| | GBL/GBL | poly-4HB | Rate* |
| Sample ID | Dimer | pyrolized | (% Wt loss/min) |
| Poly-4HB pure polymer | 14.7 | 8.72 × 10⁶ | — |
| Dry poly-4HB broth | 26.5 | 1.37 × 10⁷ | −79.7 |
| Dry poly-4HB broth + | 54.0 | 1.51 × 10⁷ | −107 |
| 5% by wt lime | | | |

*Measured from the slope of the TGA weight loss curves at 300° C. under N₂ atmosphere.

### Example 10

### Effect of Temperature, Catalyst Type, Catalyst Concentration and Broth Type on the Purity of Gamma-Butyrolactone from the Pyrolysis of a Genetically Engineered Microbe Producing Poly-4-hydroxybutyrate

In this example, a designed experiment (DOE) was carried out to determine the effects of pyrolysis temperature, catalyst type, catalyst concentration and broth type on the purity of GBL produced from a P4HB-containing microbial fermentation broth. Table 23 shows the DOE parameters and conditions tested. Sixteen different experimental conditions were tested in total. Py-GC-MS was used to measure the GBL purity. Two replicates at each condition were carried out for a total of thirty-two Py-GC-MS runs. TGA was also measured to assess the effect of the catalysts on the thermal degradation rate of P4HB at the various pyrolysis temperatures. Only single runs at each experimental condition were made for these measurements. For comparision, dry broth+P4HB samples (washed and unwashed) having no catalyst added were also prepared and analyzed by TGA and Py-GC-MS but were not part of the overall experiment.

TABLE 23
Design of Experiment parameters and conditions for determining
the effect of pyrolysis temperature, catalyst type, catalyst concentration
and broth type on GBL purity generated from microbial fermentation
broth + P4HB.

| | | Catalyst | Pyrolysis |
|---|---|---|---|
| Broth Type | Catalyst type | Concentration* | Temp (° C.) |
| Unwashed | Ca(OH)₂, | 1, 3, 5, 10% | 225, 250, 275, 300 |
| | Mg(OH)₂, FeSO₄, | | |
| | Na₂CO₃ | | |
| Washed | Ca(OH)₂, | 1, 3, 5, 10% | 225, 250, 275, 300 |
| | Mg(OH)₂, FeSO₄, | | |
| | Na₂CO₃ | | |

*Wt % metal ion relative to the dry cell mass of the broth.

Biomass containing poly(4-hydroxybutyrate) (poly-4HB) was produced in a 20 L New Brunswick Scientific fermentor (BioFlo 4500) using a genetically modified E. coli strain specifically designed for high yield production of poly-4HB from glucose syrup as a carbon feed source. Examples of the E. coli strains, fermentation conditions, media and feed conditions are described in U.S. Pat. Nos. 6,316,262; 6,689,589; 7,081,357; and 7,229,804. The E. coli strain generated a fermentation broth which had a PHA titer of approximately 100-120 g of PHA/kg of broth. After fermentation, the fermentation broth containing the microbial biomass and P4HB polymer was split into two fractions. One fraction was used without any further processing and was identified as ‘unwashed’ broth. The unwashed broth had a dry solids content of 13.7% (dry solids weight was measured using an MB-45 Ohaus Moisture Analyzer). The other fraction was washed by adding an equal volume of distilled-deionized water to the broth, stirring the mixture for 2 minutes, centrifuging and then decanting the liquid and retaining the solid biomass+P4HB. The wash step was repeated a second time and then after centrifuging and decanting, the remaining solids were resuspended again in DI water to give a 12.9% by weight dry solids solution. This material was designated ‘washed’ broth. Table 24 shows the trace metals analysis by Ion Chromatography of the two broth types. The results showed that the unwashed broth had high levels of potassium and sodium ions present due to the media components used to grow the microbial cells. After the washing step, the potassium, magnesium and sodium ions were significantly reduced thereby reducing the overall metals content of the broth+P4HB by a factor of 6.

TABLE 24
Summary of Ion Chromatography results for fermentation
broth + P4HB before and after washing with distilled
deionized water.

| | | Metal Ion |
|---|---|---|
| Broth + P4HB Type | Metal Ion | Concentration |
| Unwashed | Calcium | 39.8 ppm |
| | Magnesium | 811 ppm |
| | Potassium | 6.07% |
| | Sodium | 0.38% |
| Washed (2 times) | Calcium | 40.2 ppm |
| | Magnesium | 419 ppm |
| | Potassium | 0.83% |
| | Sodium | None detected |

The pyrolysis catalysts used in this experiment included Ca(OH)₂ (95+% Sigma Aldrich), Mg(OH)₂ (Sigma Aldrich), FeSO₄ 7H₂O (JT Baker), and Na₂CO₃ (99.5+% Sigma Aldrich). Aqueous slurries of the Ca(OH)₂, Mg(OH)₂ and FeSO₄7H₂O catalysts were prepared in DI water (25-30% by weight solids) and added to the broth samples while the Na₂CO₃ was added to the broth+P4HB directly as a solid. As shown in Table 23, the catalyst concentrations targeted for the experiment were 1%, 3%, 5% and 10% based on the weight of the metal ion relative to the dry solids weight of the broth. To prepare the broth+P4HB/catalyst samples, 10 g of either washed or unwashed broth was added to a 15 ml centrifuge tube. Next, the appropriate amount of catalyst solution or solid was added and the mixture vortexed for 30 sec. The mixture was then centrifuged, decanted and poured into a drying dish. Finally the drying dish was placed in an oven at 110° C. and dried to constant weight. Dry samples of unwashed and washed broth containing no catalysts were also prepared by centrifuging, decanting and drying at 110° C.

Table 25 shows results from the TGA and Py-GC-MS analyses on the broth+P4HB samples which have no catalysts added.

TABLE 25
Summary of TGA and Py-GC-MS results for broth + P4HB samples
having no catalyst added to them.

| Broth | | Pyrolysis | TGA Slope | GBL/GBL Dimer |
|---|---|---|---|---|
| Type | Catalyst | Temp. (° C.) | (% Wt loss/min) | Peak Area Ratio |
| unwashed | None | 225 | −17.9 | 45.6 |
| washed | None | 225 | −1.88 | 32.2 |
| unwashed | None | 250 | −43.9 | 23.1 |
| washed | None | 250 | −4.38 | 32.4 |
| unwashed | None | 275 | −64.0 | 36.6 |
| washed | None | 275 | −8.39 | 39.2 |
| unwashed | None | 300 | −97.0 | 28.9 |
| washed | None | 300 | −28.9 | 40.3 |

The results from Table 25 show that washing the broth+P4HB before pyrolyzing had a significant impact on lowering the rate of thermal decomposition at all pyrolysis temperatures. From the Ion Chromatography results in Table 24, it can be seen that the overall concentration of metal ions present in the washed broth was lowered by a factor of 6 as compared to the unwashed broth. This indicated that the metal ions present in the broth+P4HB after a fermentation run, by themselves had a catalytic effect on the degradation rate of P4HB during pyrolysis. Kim et al (2008, Polymer Degradation and Stability, 93, p776-785) have shown that the metal ions Ca, Na, Mg, Zn, Sn and Al are all effective in catalyzing the thermal degradation of P4HB. What was not shown however was the effect that these metal ions had on the purity of the GBL produced by thermal decomposition of P4HB. Table 25 shows that for the unwashed broth+P4HB samples, the GBL purity (GBL/GBL dimer peak area ratio) decreased as the pyrolysis temperature increased. For the washed samples, the purity marginally improved with increasing pyrolysis temperature. The data in Table 25 suggests that for any process making biobased GBL by thermal decomposition of P4HB and a cataylst, there exits a trade off between speed of reaction and purity of the final product. The following data will show that the type and concentration of catalyst used significantly impacts both the thermal degradation rate and GBL purity in unanticipated ways.

Table 26 summarizes the TGA and Py-GC-MS experimental results for the pyrolysis of broth+P4HB as a function of catalyst type, concentration, pyrolysis temperature and broth type.

TABLE 26
Summary of TGA and Py-GC-MS results for broth + P4HB as a function of
catalyst type, catalyst concentration, pyrolysis temperature and broth type.

| | | | Catalyst | | TGA Slope | GBL/GBL |
|---|---|---|---|---|---|---|
| | Broth | | Concentration | Pyrolysis | (% Wt | Dimer Peak |
| Run# | Type | Catalyst | (Wt %)* | Temp. (° C.) | loss/min) | Area Ratio |
| 1 | unwashed | FeSO₄ | 1% | 225 | −1.07 | — |
| 2 | unwashed | FeSO₄ | 1% | 225 | — | — |
| 3 | unwashed | Na₂CO₃ | 10% | 225 | −77.6 | 142.9 |
| 4 | unwashed | Na₂CO₃ | 10% | 225 | — | 91.74 |
| 5 | washed | Ca(OH)₂ | 3% | 225 | −35.0 | 480.7 |
| 6 | washed | Ca(OH)₂ | 3% | 225 | — | 617.3 |
| 7 | washed | Mg(OH)₂ | 5% | 225 | −33.1 | 147.6 |
| 8 | washed | Mg(OH)₂ | 5% | 225 | — | 122.1 |
| 9 | unwashed | Mg(OH)₂ | 1% | 250 | −41.6 | 38.19 |
| 10 | unwashed | Mg(OH)₂ | 1% | 250 | — | 49.75 |
| 11 | unwashed | Ca(OH)₂ | 10% | 250 | −78.2 | 1546 |
| 12 | unwashed | Ca(OH)₂ | 10% | 250 | — | 2016 |
| 13 | washed | Na₂CO₃ | 3% | 250 | −111 | 36.11 |
| 14 | washed | Na₂CO₃ | 3% | 250 | — | 28.30 |
| 15 | washed | FeSO₄ | 5% | 250 | −0.918 | — |
| 16 | washed | FeSO₄ | 5% | 250 | — | — |
| 17 | washed | Ca(OH)₂ | 1% | 275 | −14.2 | 35.39 |
| 18 | washed | Ca(OH)₂ | 1% | 275 | — | 55.07 |
| 19 | washed | FeSO₄ | 10% | 275 | −1.17 | — |
| 20 | washed | FeSO₄ | 10% | 275 | — | — |
| 21 | unwashed | Mg(OH)₂ | 3% | 275 | −109 | 118.1 |
| 22 | unwashed | Mg(OH)₂ | 3% | 275 | — | 135.2 |
| 23 | unwashed | Na₂CO₃ | 5% | 275 | −185 | 29.81 |
| 24 | unwashed | Na₂CO₃ | 5% | 275 | — | 30.84 |
| 25 | washed | Na₂CO₃ | 1% | 300 | −172 | 23.53 |
| 26 | washed | Na₂CO₃ | 1% | 300 | — | 17.33 |
| 27 | washed | Mg(OH)₂ | 10% | 300 | −55.5 | 48.59 |
| 28 | washed | Mg(OH)₂ | 10% | 300 | — | 25.52 |
| 29 | unwashed | FeSO₄ | 3% | 300 | −12.5 | — |
| 30 | unwashed | FeSO₄ | 3% | 300 | — | — |
| 31 | unwashed | Ca(OH)₂ | 5% | 300 | −164 | 46.49 |
| 32 | unwashed | Ca(OH)₂ | 5% | 300 | — | 34.45 |

*Wt % metal ion relative to the dry solids weight of the broth.

Statistical analysis of the data in Table 26 (using JMP statistical software from SAS), showed that for the fastest thermal degradation rate, the optimum variable parameters to use would be unwashed broth+P4HB, Na₂CO₃ as the catalyst at 5% concentration and a pyrolysis temperature of 300° C. Catalyst type was the most significant variable affecting the degradation rates which varied from −1 to −185% wt loss/min. Samples with FeSO₄ catalyst had degradation rates lower than even the washed broth+P4HB indicating that this compound acted more as a P4HB thermal stabilizer rather than a catalyst promoter. The samples which had the highest degradation rates were those with either Na₂CO₃ or Ca(OH)₂. Higher temperatures and generally higher catalyst concentration also favored faster degradation rates.

The statistical analysis of the GBL purity data showed that the optimum variable parameters for highest GBL purity were found using Ca(OH)₂ catalyst at 10% concentration and a pyrolysis temperature of 250° C. In comparison to the other variables, broth type had a negligible effect on the GBL purity. The most statistically significant variables for GBL purity, which ranged in value from 17 to 2016 (GBL/GBL dimer peak area ratio) were catalyst concentration and type. It was noted that the upper range values for GBL purity in the experimental results were much higher than those observed for the unwashed broth+P4HB samples in Table 25. This indicated that the metal ions remaining in broth from fermentation (mostly potassium) were not as effective for improving GBL purity as those used in the experiment. Pyrolysis tempertature was also found to be a statistically significant variable for GBL purity (higher temperatures generated more dimer). In Table 26, the missing Py-GC-MS data for broth+P4HB with FeSO₄ as the catalyst was due to the fact that the samples took too long to pyrolyze under the Py-GC-MS conditions and therefore could not be quantitated. This was in agreement with the TGA data which showed FeSO₄ acted as a thermal stabilizer rather than catalyst promoter.

As shown in Example 9, addition of the catalyst Ca(OH)₂ to microbial biomass+P4HB suppressed the formation of GBL dimer producing a purer GBL liquid during pyrolysis of the biomass. The above experimental data confirmed this observation and showed that cataylst concentration and pyrolysis temperature were also important in determining the optimum conditions for producing high purity GBL from dry broth+P4HB by pyrolysis. The choice of catalyst and pyrolysis temperature was also shown to impact the rate of P4HB thermal degradation. Therefore one needs to carefully choose the correct conditions to optimize both variables when designing a robust process for production of biobased GBL.

### Example 11

### Larger Scale Production of Gamma-Butyrolactone from the Pyrolysis of a Genetically Engineered Microbe Producing Poly-4-hydroxybutyrate

In the following example, GBL production from pyrolyis of a fermentation broth+P4HB+catalyst mixture will be outlined showing the ability to produce a high purity, high yield biobased GBL on the hundred gram scale.

Biomass containing poly-4-hydroxybutyrate (poly-4HB) was produced in a 20 L New Brunswick Scientific fermentor (BioFlo 4500) using a genetically modified E. coli strain specifically designed for high yield production of poly-4HB from glucose syrup as a carbon feed source. Examples of the E. coli strains, fermentation conditions, media and feed conditions are described in U.S. Pat. Nos. 6,316,262; 6,689,589; 7,081,357; and 7,229,804. The E. coli strain generated a fermentation broth which had a PHA titer of approximately 100-120 g of PHA/kg of broth. After fermentation, the broth was washed with DI water by adding an equal volume of water, mixing for 2 minutes, centrifuging and decanting the water. Next, the washed broth was mixed with lime (Ca(OH)₂ standard hydrated lime 98%, Mississippi Lime) targeting 4% by wt dry solids. The mixture was then dried in a rotating drum dryer at 125-130° C. to a constant weight. Moisture levels in the dried biomass were approximately 1-2% by weight. The final wt % calcium ion in the dried broth+P4HB was measured by Ion Chromatography to be 1.9% (3.5% by wt. Ca(OH)₂).

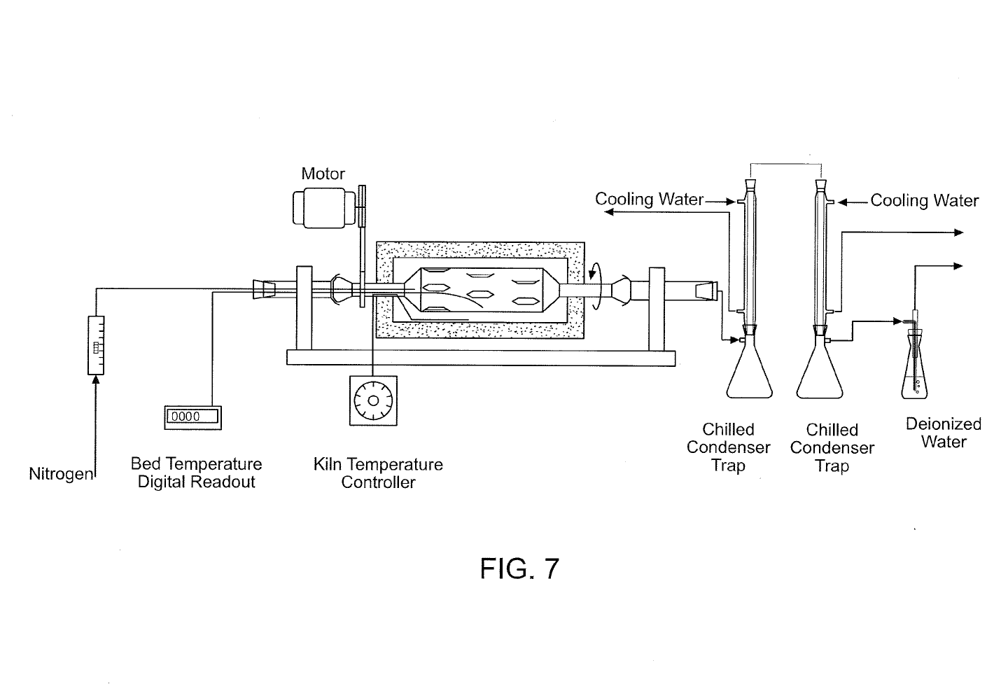

Pyrolysis of the dried broth+P4HB+Ca(OH)₂ was carried out using a rotating, four inch diameter quartz glass kiln suspended within a clamshell tube furnace. At the start of the process, a weighed sample of dried broth+P4HB+Ca(OH)₂ was placed inside of the glass kiln and a nitrogen purge flow established. The furnace rotation and heat up would then be started. As the temperature of the furnace reached its set point value, gases generated by the broth+P4HB+Ca(OH)₂ sample would be swept out of the kiln by the nitrogen purge and enter a series of glass condensers or chilled traps. The condensers consisted of a vertical, cooled glass condenser tower with a condensate collection bulb located at the its base. A glycol/water mixture held at 0° C. was circulated through all of the glass condensers. The cooled gases that exited the top of the first condenser were directed downward through a second condenser and through a second condensate collection bulb before being bubbled through a glass impinger filled with deionized water. FIG. 7 shows a schematic diagram of the pyrolyzer and gas collection equipment.

For the larger scale pyrolysis experiment, 292 g of dried broth+P4HB+Ca(01-1)₂ was first loaded into the quartz kiln at room temperature. The total weight of P4HB biomass was estimated to be 281.4 g based on Ca(OH)₂ loading. The wt % P4HB in the mixture was also measured to be 66.7% (see Doi, Microbial Polyesters, John Wiley and Sons, p23, 1990) based on the dry solids which made the mass of P4HB in the kiln equal to 195 g. The system was then sealed up and a nitrogen purge of approximately 1500 ml/min was established. Power was applied to the furnace and the dried broth+P4HB+Ca(OH)₂ was heated up to the target pyrolysis temperature of 250° C. During pyrolysis, the products of thermal degradaton of biomass+P4HB, GBL, were collected in the condensate traps below the cooled condensers. Water could be seen to collect initially in each of the collection bulbs. The majority of the liquified product (>95%) was collected in the first glass collection bulb. Total pyrolysis run time was aproximately 60 minutes. The weight of the remaining biomass after pyrolysis was measured to be 11.9 g.

After the completion of the pyrolysis run, the condensates from the condensers were collected and weighed. The results showed that the combined condensate weight was 181 g. Analysis of the condensate by Karl Fisher and GC-MS showed that the condensate contained 6.1% water, 0.06% fatty acids with the balance of the material being GBL products. The GBL product yield ((g of GBL product/g of starting P4HB)×100%) therefore was calculated to be approximately 87%. The GC-MS results also showed that the major impurity in the GBL product was GBL dimer where the peak area ratio of GBL/GBL dimer was calculated to be 2777. This was in agreement with the results from the design of experiments in Example 10 showing that the optimum process conditions for highest GBL purity were at the 250° C. pyrolysis temperature with the Ca(OH)₂ catalyst. Other impurities such as organosulfur and amide compounds were also detected as being present in the condensate by GC-MS. The conversion of the P4HB biomass solid to liquid ((g of dry Biomass—g Residual biomass/g of dry biomass)×100%) was calculated to be 96%.

The processing of fats and oils to produce alcohols provides some guidance in this respect. Oils and fats are significant sources of fatty alcohols that are used in a variety of applications such as lubricants and surfactants. The fats are not typically hydrogenated directly as the intensive reaction conditions tend to downgrade the glycerol to lower alcohols such as propylene glycol and propanol during the course of the hydrogenation. For this reason it is more conventional to first hydrolyze the oil and then pre-purify the fatty acids to enable a more efficient hydrogenation (see for instance Lurgi's hydrogenation process in Bailey's Industrial Oil and Fat Products, Sixth Edition, Six Volume Set. Edited by Fereidoon Shahidi, John Wiley & Sons, Inc. 2005).

### Example 12

### Direct Generation of Biobased Tetrahydrofuran (THF) from the Pyrolysis of a Genetically Engineered Biomass Producing Poly-4-hydroxybutyrate

In the following example, THF production from pyrolyis of a fermentation broth+P4HB+catalyst mixture will be outlined indicating the ability to produce THF as the main product from biomass containing P4HB. Biomass containing poly-4HB is produced in a 20 L New Brunswick Scientific fermentor (BioFlo 4500) using a genetically modified E. coli strain specifically designed for high yield production of poly-4HB from glucose syrup as a carbon feed source. Examples of the E. coli strains, fermentation conditions, media and feed conditions are described in U.S. Pat. Nos. 6,316,262; 6,689,589; 7,081,357; and 7,229,804. The E. coli strain generates a fermentation broth which has a PHA titer of approximately 100-120 g of PHA/kg of broth. After fermentation, the broth is mixed with calcium hydroxide (Ca(OH)₂ standard hydrated lime 98%, Mississippi Lime) targeting 4% by wt dry solids. The mixture is then dried in a rotating drum dryer at 125-130° C. to a constant weight. Moisture levels in the dried biomass are targeted to approximately 1-2% by weight. The final wt % calcium ion in the dried broth+P4HB was measured by Ion Chromatography to be 1.9% (3.5% by wt. Ca(OH)₂).

Pyrolysis of the dried broth+P4HB+Ca(OH)₂ is carried out using a rotating, four inch diameter quartz glass kiln suspended within a clamshell tube furnace. At the start of the process, a weighed sample of dried broth+P4HB+Ca(OH)₂ is placed inside of the glass kiln and a nitrogen purge flow established. As the temperature of the furnace reaches its set point value, gases generated by the broth+P4HB+Ca(OH)₂ sample are swept out of the kiln by the nitrogen flow and passed over a partial condenser. The condenser temperature is controlled such that very little GBL condenses along with the high boiling pyrolysis oils that are removed from the bottom of the condenser. The GBL vapors are then passed over a guard bed that contains an absorbent designed to remove nitrogen containing impurities that would otherwise poison the hydrogenation catalyst. The absorbent could be a composed of clay, alumina, zeolite, silica gel and activated carbon as outlined in U.S. Pat. No. 7,867,381. The polished GBL vapor is then compressed to 5 bars and mixed with hydrogen gas at temperature of 190° C. to obtain a gas mixture with a composition of 1% by volume GBL and 99% hydrogen that is then fed to the catalytic reactor at a GHSV of 3000 hr⁻¹ (Gas hourly space velocity=volume flow of gas at standard temperature and pressure divided by bed volume of catalyst).

The hydrogentation catalyst used in the reactor is produced by mixing 40% by weight of CuO, 40% by weight of ZnO and 20% by weight of Al₂O₃ with uniformly sized glass rings that is then place in the tube reator. Before introducing the GBL vapor to the reactor, the catalyst is first activated by flushing with nitrogen over a period of 1 hour at a temperature of 180° C. Thereafter hydrogen is gradually introduced without exceeding a temperature of 250 C until only hydrogen and no nitrogen is flowing over the catalyst bed at a temperature less than 190° C. Thereafter the GBL and hydrogen reaction mixture is introduced to the bed.

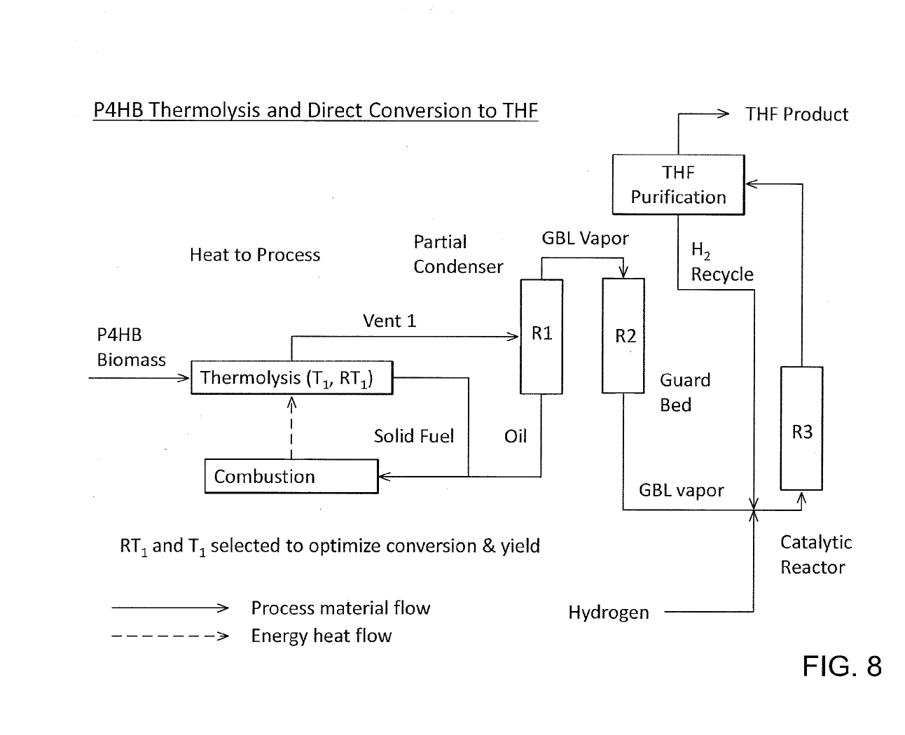

All the GBL in the feed is completely converted with more than 99% THF selectivity. This gas mixture is then condensed to produce THF liquid that is separated from the hydrogen which can be recycled to the reactor together with fresh make-up hydrogen gas. FIG. 8 shows a schematic diagram of the P4HB biomass to THF conversion process as outlined above.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A process for production of a biobased tetrahydrofuran product, comprising:
a) combining a genetically engineered biomass and a first catalyst, wherein the biomass comprises a poly-4-hydroxybutyrate;
b) heating the biomass with the first catalyst to convert the poly 4-hydroxybutyrate to gamma-butyrolactone vapor; wherein a yield of the gamma-butyrolactone vapor is at least 85%; and
c) hydrogenating the gamma-butyrolactone vapor using a second catalyst to produce the biobased tetrahydrofuran product, wherein the genetically engineered biomass is from a recombinant host having a poly-4-hydroxybutyrate pathway, wherein the host has an inhibiting mutation in its CoA-independent NAD-dependent succinic semialdehyde dehydrogenase gene or its CoA-independent NADP-dependent succinic semialdehyde dehydrogenase gene, or having the inhibiting mutations in both genes, and having stably incorporated one or more genes encoding one or more enzymes selected from: a succinyl-CoA:coenzyme A transferase, a succinate semialdehyde dehydrogenase, a succinic semialdehyde reductase, a CoA transferase, and a polyhydroxyalkanoate synthase, wherein the succinyl-CoA:coenzyme A transferase converts succinate to succinyl-CoA, the succinate semialdehyde dehydrogenase converts succinyl-CoA to succinic semialdehyde, the succinic semialdehyde reductase converts succinic semialdehyde to 4-hydroxybutyrate, the CoA transferase converts 4-hydroxybutyrate to 4-hydroxybutyryl-CoA and the polyhydroxyalkanoate synthase polymerizes 4-hydroxybutyryl-CoA to poly-4-hydroxybutyrate.

2. (canceled)

3. The process of claim 1, wherein the genetically engineered biomass is from a recombinant host having stably incorporated one or more genes encoding one or more enzymes selected from: a phosphoenolpyruvate carboxylase, an isocitrate lyase, a malate synthase, an ADP-forming succinate-CoA ligase, an NADP-dependent glyceraldeyde-3-phosphate dehydrogenase, an NAD-dependent glyceraldeyde-3-phosphate dehydrogenase, a butyrate kinase, and a phosphotransbutyrylase; and optionally having a disruption in one or more genes selected from yneI, gabD, pykF, pykA, maeA and maeB, wherein the phosphoenolpyruvate carboxylase converts phosphoenolpyruvate to oxaloacetate, the isocitrate lyase converts isocitrate to glyoxalate, the malate synthase converts glyoxalate to malate and succinate, the ADP-forming succinate-CoA ligase converts succinate to succinyl-CoA, the NADP-dependent glyceraldeyde-3-phosphate dehydrogenase converts glyceraldehyde 3-phosphate to 1,3-bisphosphoglycerate forming NADPH+H⁺, the NAD-dependent glyceraldeyde-3-phosphate dehydrogenase converts glyceraldehyde 3-phosphate to 1,3-bisphosphoglycerate forming NADH+H⁺, the butyrate kinase converts 4-hydroxybutyrate to 4-hydroxybutyryl-phosphate, and the phosphotransbutyrylase converts 4-hydroxybutyryl-phosphate to 4-hydroxybutyryl-CoA.

4. The process of claim 1, wherein the process further includes an initial step of culturing a recombinant host with a renewable feedstock to produce a poly-4-hydroxybutyrate biomass wherein a source of the renewable feedstock is selected from glucose, fructose, sucrose, arabinose, maltose, lactose, xylose, fatty acids, vegetable oils, and biomass derived synthesis gas or a combination thereof.

5. (canceled)

6. The process of claim 1, wherein the biomass host is a bacteria, yeast, fungi, algae, cyanobacteria, or a mixture of any two or more thereof.

7-9. (canceled)

10. The process of claim 1, wherein heating is at a temperature of from about 100° C. to about 350° C., from about 200° C. to about 350° C. or from about 225° C. to about 300° C.

11. (canceled)

12. The process of claim 11, wherein the weight percent of the first catalyst is in the range of about 4% to about 50%.

13. The process of claim 1, wherein heating reduces the water content of the biomass to about 5 wt %.

14-15. (canceled)

16. The process of claim 1, wherein the heating is for a time period from about 30 seconds to about 5 minutes or from about 5 minutes to about 2 hours.

17. (canceled)

18. The process of claim 1, wherein the gamma-butyrolactone vapor comprises less than 5% by weight of side products.

19. (canceled)

20. The process of claim 19, wherein the partially condensed gamma-butyrolactone vapor is passed through an absorbent bed.

21. (canceled)

22. The process of claim 1, wherein the gamma-butyrolactone vapor during hydrogenating is mixed with hydrogen gas at a weight ratio of hydrogen gas/GBL of 99%/1% to about 95%/5%.

23. The process of claim 22, wherein the hydrogen/GBL gas mixture is compressed to 2 to about 8 bars.

24. The process of claim 23, wherein the compressed hydrogen gas/GBL mixture is heated to 180° C. to about 210° C.

25. The process of claim 1, wherein the second catalyst is a vapor phase hydrogenation catalyst.

26. The process of claim 1, wherein the second catalyst is a mixture of 20-40% CuO, 20-40% ZnO, 5-20% Al₂O₃ by weight.

27. The process of claim 24, wherein the heated and compressed, hydrogen/GBL gas mixture is exposed to the second catalyst.

28. The process of claim 1, wherein the genetically engineered biomass is from a recombinant host having a poly-4-hydroxybutyrate pathway, wherein the host has optionally an inhibiting mutation in its CoA-independent NAD-dependent succinic semialdehyde dehydrogenase gene or its CoA-independent NADP-dependent succinic semialdehyde dehydrogenase gene, or having inhibiting mutations in both genes, and having stably incorporated genes encoding the following enzymes: a succinyl-CoA:coenzyme A transferase, a succinate semialdehyde dehydrogenase, a succinic semialdehyde reductase, a CoA transferase, and a polyhydroxyalkanoate synthase, wherein the succinyl-CoA:coenzyme A transferase converts succinate to succinyl-CoA, the succinate semialdehyde dehydrogenase converts succinyl-CoA to succinic semialdehyde, the succinic semialdehyde reductase converst succinic semialdehyde to 4-hydroxybutyrate, the CoA transferase converts 4-hydroxybutyrate to 4-hydroxybutyryl-CoA, and the polyhydroxyalkanoate synthase polymerizes 4-hydroxybutyryl-CoA to poly-4-hydroxybutyrate.

29. The process of claim 1, wherein the genetically engineered biomass is from a recombinant host having stably incorporated genes encoding the following enzymes: a phosphoenolpyruvate carboxylase, an isocitrate lyase, a malate synthase wherein the malate synthase is able to convert glyoxalate to malate and succinate, an ADP-forming succinate-CoA ligase, an NADP-dependent glyceraldeyde-3-phosphate dehydrogenase wherein the NADP-dependent glyceraldeyde-3-phosphate dehydrogenase is able to convert glyceraldehyde 3-phosphate to 1,3-bisphosphoglycerate forming NADPH+H⁺, an NAD-dependent glyceraldeyde-3-phosphate dehydrogenase, a butyrate kinase, a phosphotransbutyrylase; and optionally having a disruption in one or more genes selected from yneI, gabD, pykF, pykA, maeA and maeB, wherein the phosphoenolpyruvate carboxylase converts phosphoenolpyruvate to oxaloacetate, the isocitrate lyase converts isocitrate to glyoxalate, the succinate-CoA ligase (ADP-forming) converts succinate to succinyl-CoA, the NAD-dependent glyceraldeyde-3-phosphate dehydrogenase converts glyceraldehyde 3-phosphate to 1,3-bisphosphoglycerate forming NADH+H⁺, the butyrate kinase converts 4-hydroxybutyrate to 4-hydroxybutyryl-phosphate and the phosphotransbutyrylase converts 4-hydroxybutyryl-phosphate to 4-hydroxybutyryl-CoA.

30. The process of claim 1, wherein the genetically engineered biomass is from a recombinant host having a poly-4-hydroxybutyrate pathway, wherein the host has stably incorporated one or more genes encoding one or more enzymes selected from a succinyl-CoA:coenzyme A transferase, a succinate semialdehyde dehydrogenase, a succinic semialdehyde reductase, a CoA transferase, and a polyhydroxyalkanoate synthase wherein the succinyl-CoA:coenzyme A transferase converts succinate to succinyl-CoA, the succinate semialdehyde dehydrogenase converts succinyl-CoA to succinic semialdehyde, the succinic semialdehyde reductase converts succinic semialdehyde to 4-hydroxybutyrate, the CoA transferase converts 4-hydroxybutyrate to 4-hydroxybutyryl-CoA, and the polyhydroxyalkanoate synthase polymerizes 4-hydroxybutyryl-CoA to poly-4-hydroxybutyrate.

31. The process of claim 1, wherein the genetically engineered biomass is from a recombinant host having stably incorporated one or more genes encoding one or more enzymes selected from: a phosphoenolpyruvate carboxylase, an isocitrate lyase, a malate synthase, an ADP-forming succinate-CoA ligase, an NADP-dependent glyceraldeyde-3-phosphate dehydrogenase, an NAD-dependent glyceraldeyde-3-phosphate dehydrogenase, a butyrate kinase, a phosphotransbutyrylase; and optionally having a disruption in one or more genes selected from yneI, gabD, pykF, pykA, maeA and maeB, wherein the phosphoenolpyruvate carboxylase converts phosphoenolpyruvate to oxaloacetate, the isocitrate lyase converts isocitrate to glyoxalate, the malate synthase converts glyoxalate to malate and succinate, the ADP-forming succinate-CoA ligase converts succinate to succinyl-CoA, the NADP-dependent glyceraldeyde-3-phosphate dehydrogenase converts glyceraldehyde 3-phosphate to 1,3-bisphosphoglycerate forming NADPH+H⁺, the NAD-dependent glyceraldeyde-3-phosphate dehydrogenase converts glyceraldehyde 3-phosphate to 1,3-bisphosphoglycerate forming NADH+H⁺, the butyrate kinase converts 4-hydroxybutyrate to 4-hydroxybutyryl-phosphate, and the phosphotransbutyrylase converts 4-hydroxybutyryl-phosphate to 4-hydroxybutyryl-CoA.

32-34. (canceled)

35. The process of claim 34, wherein the tetrahydrofuran product is further processed to an elastic fiber and the elastic fiber is spandex.

36. A biobased tetrahydrofuran product produced by the process of claim 1.

37. The product of claim 36, wherein the tetrahydrofuran product comprises less than 1% by weight of side products.

38. A poly-4-hydroxybutyrate biomass produced from renewable resources which is suitable as a feedstock for producing gamma-butyrolactone product, wherein the level of poly-4-hydroxybutyrate in the biomass is greater than 50% by weight of the biomass.

39. (canceled)

40. The process of claim 1, wherein the product yield is about 95% by weight or greater based on one gram of a tetrahydrofuran in the product per gram of gamma-butyrolactone vapor.
